(19) United States
(12) Patent Application Publication
(10) Pub. No.: US 2004/0077654 A1
(43) Pub. Date: Apr. 22, 2004

(54) Aryl piperidine and piperazine derivatives as inducers of ldl-receptor expression

(76) Inventors: Anne Marie Jeanne Bouillot, Les Ulis (FR); Agnes Bombrun, Geneva (CH); Bernard Andre Dumaitre, Les Ulis (FR); Romain Luc Gosmini, Les Ulis (FR); Miles Stuart Congreve, Cambridge (GB); Nigel Grahame Ramsden, Hertfordshire (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/250,713

(22) PCT Filed: Jan. 15, 2001
(86) PCT No.: PCT/GB01/00158

(51) Int. Cl. A61K 31/496; A61K 31/4178

(52) U.S. Cl. 514/252.13; 514/397

(57) ABSTRACT

The invention concerns Use of a compound of formula (I) or a physiologically acceptable salt, solvate or derivative thereof, in the manufacture of a medicament for the treatment of diseases ameliorated by LDL-r upregulation, to novel compounds and pharmaceutical compositions within the scope of formula (I).

This invention relates to novel compounds which up-regulate LDL receptor (LDL-r) expression and to processes for their preparation, pharmaceutical compositions containing them and their medical use. More particularly, this invention relates to novel aromatic piperidines and piperazines and their use in therapy.

Epidemiological studies have clearly demonstrated the correlation between reduction in plasmatic LDL cholesterol and the benefit on cardiovascular events including mortality. LDL cholesterol is eliminated from plasma by specific binding to LDL-r expressed by the liver. Regulation of LDL-r expression occurs in the liver- and is mainly dependent on intracellular cholesterol concentration. Increasing free cholesterol concentration leads to a reduced LDL-r expression through a mechanism involving transcriptional factors. Counteracting with this process is expected to up-regulate LDL-r expression in the liver and to increase the clearance of LDL cholesterol.

International Patent Application Number PCT.EP00.06668 concerns the novel use of the SREBP-cleavage activating protein (SCAP) in a screening method, and two compounds are disclosed, namely 4-(4-chloro-benzoylamino)-N-{4-[4-(2-ethoxy-4-ethyl-phenyl)-piperidin-1-yl]-butyl}-benzamide and 4-(4-Benzoyl)-N-{4-[4-(4-isopropyl-2-methoxy-phenyl)-piperidin-1-yl]-butyl}-benzamide hydrochloride, which do not form part of the present invention.

Another publication, Bioorganic and Medicinal Chemistry Letters Vol. 5, 3, 219-222, 1995 discloses compounds having the general formula (A)

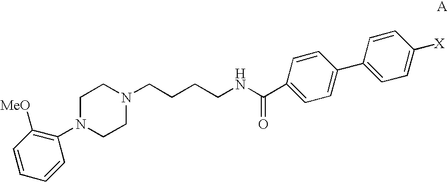

where X may be COMe, SO₂Me and NH₂, as having high affinity for the dopamine D₃ receptor and postulates their use in CNS disorders, particularly psychiatric illness. The compound of formula A where X is COMe is also disclosed in J. Pharmacol. Exp. Ther. 287; 1 1998 187-197 and Bioorganic and Medicinal Chemistry Letters Vol. 7, 15, 1995-1998, 1997, again as being useful in treating CNS disorders. It will be noted that the examples of the present invention differ from those of formula (A) in the utility disclosed.

Journal Of Medicinal Chemistry, Vol. 40, 6, 952-960, 1997 discloses compounds of formula (B)

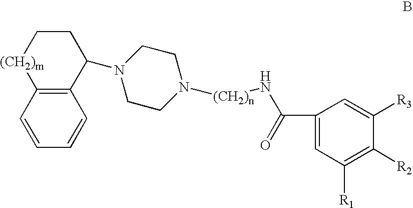

where m=0, 1 or 2; n-2 or 3; R₁ and R₃═H or OMe and R² may be Ph, as selective 5-HT_{1A} receptor ligands having CNS activity. It will be noted that the examples of the present invention differ from those of formula (B) in the utility disclosed.

International Patent Application Publication Number WO99/45925 discloses compounds of formula (C)

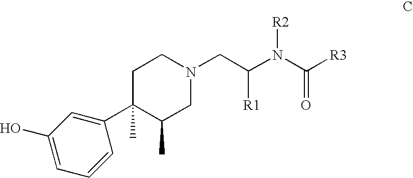

where R1 may be hydrogen, R2 may be hydrogen and R3 may be a group

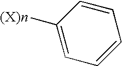

where X may be an aryl group and n may be 1. Specifically disclosed are compounds where the group COR3 is formed from 2- and 4-biphenyl carboxylic acid and R1 and R2 are methyl or hydrogen respectively. The utility of the compounds is as opioid receptor binding agents which may be useful as analgesics. The substitution on the 3- and 4-positions of the piperidine ring leave the compounds of this publication outside the scope of the present invention. Furhtermore, the utility disclosed is different.

International Patent Application Publication Number WO98/37893 discloses compounds of formula (D)

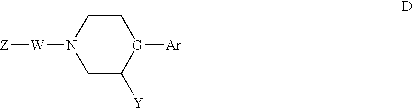

where Ar may represent an optionally substituted phenyl or naphthyl, G may be N or CH₂ (sic), W may be an optionally substituted alkylene, Y may be hydrogen and Z may represent a group R₄CONR₅, where R₄ may be an optionally substituted phenyl and R₅ may be hydrogen. These compounds are described as being D2 receptor antagonists useful in the treatment of CNS disorders such as Parkinson's Disease. None of the compounds specifically disclosed fall within the scope of the present invention and the disclosed utlity is different.

International Patent Application Publication Number WO9402473 discloses compounds of formula (E)

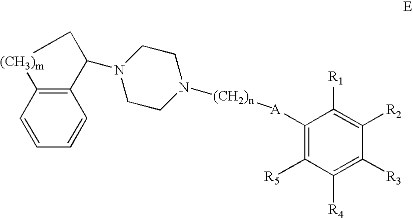

where A may be NHCO or CONH; R₁-R₅ may be hydrogen or a benzene ring, m may be 1-3 and n may be 1-3. Specifically disclosed are compounds

| No. | A | n | m | R₁ | R₂ | R₃ | R₄ | R₅ |
|---|---|---|---|---|---|---|---|---|
| 5 | NHCO | 2 | 1 | H | H | Ph | H | H |
| 12 | NHCO | 2 | 2 | H | H | Ph | H | H |
| 19 | NHCO | 2 | 3 | H | H | Ph | H | H |

The compounds are described as 5HT-1A agonists having CNS activity and may be used as anti-depressants, anti-hypertensive, analgesics etc. It will be noted that the examples of the present invention differ from those of formula (E) in the utility disclosed.

International Patent Application Publication Number WO99/45925 discloses compounds of formula (F)

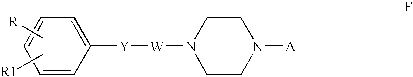

where A may represent a substituted phenyl group, W represents a linear or branched alkylene group having from 2 to 6 carbon atoms; Y may represent a group NHCO or CONH; and R may be a substituted phenyl group. Particularly disclosed is the compound G

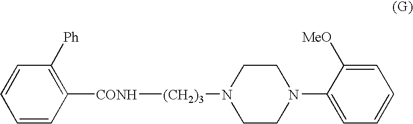

These compounds are described as being α1A-adrenergic receptors useful in the treatment of contractions of the prostate, urethra and lower urinary tract, without affecting blood pressure. It will be noted that the examples of the present invention differ from those of formula (G) in the utility disclosed.

International Patent Application Publication Number WO98/35957 describes compounds of formula (H)

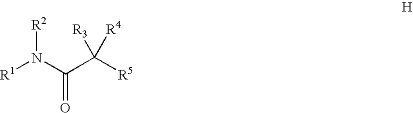

wherein R1-R5 are each individually selected from the group of substituents including hydrogen, halogen, hydroxyl, thiol, lower alkyl, substituted lower alkyl, alkenyl, alkynyl, alkylalkenyl, alkylalkynyl, alkoxy, alkylthio, acyl, aryloxy, amino, amido, carboxyl, aryl, substituted aryl, heterocycle, heteroaryl, substituted heterocycle, heteroalkyl, cycloalkyl, substituted cycloalkyl, alkylcycloalkyl, alkylcycloheteroalkyl, nitro and cyano. Specifically disclosed compounds are those formed by the N-alkylation of a a substituted piperidine or piperazine with a group (J)

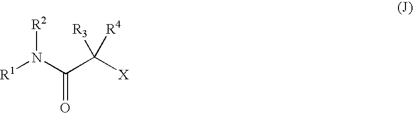

where X is a leaving group. None of the compounds specifically disclosed fall within the scope of the present invention and the invention is in no way suggested by the disclosure. The compounds are said to be of use as NPY Y5 receptor antagonists in the treatment of obesity, bulemia and related disorders and NPY Y5 receptor inhibition related disorders such as memory disorders, epilepsy, dyslipidemia and depression. U.S. Pat. No. 6,048,900, published after the priority date of the present invention discloses the same information.

Journal Of Medicinal Chemistry, Vol. 31, 1968-1971, 1988 discloses certain aryl piperazines compounds, which fall outside the present invention, as 5HT-1a Serotonin Ligands as potential CNS agents. Specifically disclosed are compounds of formula (K)

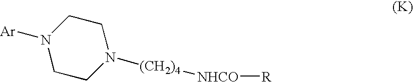

where Ar=Ph and R=Ph, Ar=2-OMePh and R=Ph and Ar=2-pyrimidyl and R=Ph.

Journal Of Medicinal Chemistry, Vol. 34, 2633-2638, 1991 discloses aryl piperazines having reduced α1 adrenergic affinity. Specifically disclosed is the compound (L)

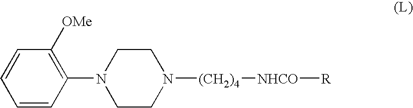

where R is 4-(BnO)-phenyl, which falls outside the scope of the present invention.

Thus, as a first aspect, the present invention provides the use of a compound of formula (I)

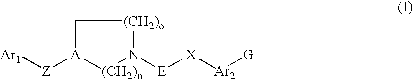

wherein

Ar₁ represents

(i) phenyl, naphthyl, or phenyl fused by a C₃₋₈cycloalkyl,

(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,

where Ar₁ optionally optionally bears 1-4 groups independently represented by R¹;

R¹ is selected from halogen, —S(C₁₋₄alkyl), —O—(C₁₋₄ alkylene)-R² or —(C₀₋₄alkylene)-R², where each alkylene group may additionally incorporate an oxygen in the chain, with the proviso that there are at least two carbon atoms between any chain heteroatoms;

R² represents

(i) hydrogen, C₁₋₄ perfluoroalkyl, C₁₋₄perfuoroalkoxy,

(ii) phenyl, phenyl fused by a C₃₋₈cycloalkyl, naphthyl or a 5- or 6-membered heteroaromatic group, optionally substituted by one or two groups independently selected from halogen, C₁₋₄alkyl, hydroxy, C₁₋₄ alkoxy, amino, C₁₋₄ alkylamino and di-C₁₋₄alkylamino,

(iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-C₁₋₄ alkylamino, or

(iv) amino, C₁₋₄ alkylamino or di-C₁₋₄alkylamino, with the proviso that there are at least two carbon atoms between any chain heteroatoms;

Z is a direct link, oxo, —O—, C(H)R³, —N(R⁵)—, —N(SO₂R⁶)— or —SO₂—;

R³ is hydrogen, C₁₋₄ alkyl or phenyl, said phenyl optionally bearing one or two groups independently selected from halogen, C₁₋₄ alkyl, C₁₋₄ alkoxy and OH;

A is C—R⁴ or N;

n is an integer selected from 1-3;

o is an integer selected from 1-2;

R⁴ is hydrogen, C₁₋₄ alkyl, hydroxy or phenyl, said phenyl optionally bearing one or two groups indpendently selected from halogen, C₁₋₄ alkyl, C₁₋₄ alkoxy or hydroxy, or R³ forms a double bond between A and an adjacent ring carbon;

R⁵ is C₁₋₄ alkyl or phenyl;

R⁶ is C₁₋₄ alkyl or phenyl;

E is a C₁₋₆ alkylene group, optionally containing one or two double bonds or one triple bond and optionally incorporating an O, S or N(H or C₁₋₄ alkyl) group in the chain;

X is a direct link, —O—, oxo, —CON(H or C₁₋₄ alkyl)-, —N(H or C₁₋₄ alkyl)CO—, —N(H or C₁₋₄ alkyl)SO₂— or —SO₂N(H or C₁₋₄ alkyl)-;

Ar₂ is phenyl, a 56 membered heteroaromatic group or a bicyclic heteroaromatic group, where each group is optionally substituted by one or two groups independently selected from C₁₋₄ alkyl, halogen, hydroxy, C₁₋₄ alkoxy, C₁₋₆ acyl, C₁₋₆ acyloxy, amino, C₁₋₄ alkylamino and di-C₁₋₄ alkylamino groups;

G is hydrogen or —Y—Ar₃;

Y is a direct link, oxo, —O—, —N(H or C₁₋₄ alkyl)CO—, —CON(H or C₁₋₄ alkyl)-, —N(H or C₁₋₄ alkyl)SO₂— or —SO₂N(H or C₁alkyl)-, —C₁₋₂ alkylene-, —O—C₁₋₂ alkylene- or C₂₋₃alkenylene-;

Ar₃ represents

(i) phenyl, naphthyl, or phenyl fused by a C₃₋₈cycloalkyl,

(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,

where Ar₃ optionally bears 1-4 groups independently selected from hydroxy, alkyl, C₁₋₄ alkoxy, C₂₋₄ alkenyl, C₂₋₄ alkenyloxy, C₁₋₄ perfluoroalkoxy, C₁₋₄ acylamino or an electron withdrawing group;

or a physiologically acceptable salt, solvate or derivative thereof, in the manufacture of a medicament for the treatment of diseases ameliorated by LDL-r upregulation.

As an alternative aspect, the invention provides a method of treatment of a mammal of diseases ameliorated by LDL-r upregulation, comprising administration of an effective amount of a compound of formula (I) or a physiologically acceptable salt, solvate or derivative thereof.

As a further or alternative aspect, the invention provides the use of a compound of formula (I), as described hereinabove, in the manufacture of a medicament for the treatment of diseases ameliorated by LDL-r upregulation, with the proviso that 4-(4-chloro-benzoylamino)-N-{4-[4-(2-ethoxy-4-ethyl-phenyl)-piperidin-1-yl]-butyl}-benzamide and 4-(4-Benzoyl)-N-{4-[4-(4-isopropyl-2-methoxy-phenyl)-piperidin-1-yl]-butyl}-benzamide are not included.

Suitable physiologically acceptable salts of the compounds of general formula (I) include acid addition salts formed with pharmaceutically acceptable inorganic acids for example, hydrochlorides, hydrobromides or sulphates, or with pharmaceutically acceptable organic acids for example mesylates, lactates and acetates. More suitably, a physiologically acceptable salt of the compounds of general formula (I) is a mesylate salt.

The solvates may, for example, be hydrates.

References hereinafter to a compound according to the invention include both compounds of formula (I) and their physiologically acceptable salts together with physiologically acceptable solvates.

The term “physiologically acceptable derivative” as used herein refers to any physiologically acceptable derivative of a compound of the present invention, for example, an ester, which upon administration to a mammal, such as a human, is capable of providing (directly or indirectly) such a compound or an active metabolite thereof. Such derivatives are clear to those skilled in the art, without undue experimentation, and with reference to the teaching of Burger's Medicinal Chemistry And Drug Discovery, 5th Edition, Vol 1: Principles And Practice, which is incorporated herein by reference. Suitable ester groups include the groups —O—R⁷, where R⁷ may represent C₁₋₄ acyl, C₁₋₄ acyloxymethylene, optionally substituted benzoyl, where optional substitution may be effected by one or more C₁₋₄ alkyl, halogen, hydroxy or C₁₋₄ alkoxy, —PO(OR⁸)₂, where R⁸ represents hydrogen, C₁₋₄ alkyl, phenyl or phenylmethyl, carboxyethylcarbonyl, C₁₋₄ alkylaminocarbonyl, C₁₋₄ dialkylaminocarbonyl or esters formed with readily available amino acids, e.g. dimethylaminomethylcarbonyl. R⁷ is more sutiably C₁₋₄ acyl, e.g. acetyl or —PO(OR⁸)₂, where R⁸ represents hydrogen, C₁₋₄ alkyl, phenyl or phenylmethyl, e.g. phosphate.

Referring to the general formula (I), alkyl, alkylene and alkoxy include both straight and branched chain saturated hydrocarbon groups. Examples of alkyl groups include methyl and ethyl groups, examples of alkylene groups include methylene and ethylene groups, whilst examples of alkoxy groups include methoxy and ethoxy groups.

Referring to the general formula (I), acyl refers to aliphatic or cyclic hydrocarbons attached to a carbonyl group through which the substituent bonds, such as acetyl.

Referring to the general formula (I), phenyl fused by a C₃₋₈cycloalkyl includes bicyclic rings such as 1,2,3,4-tetrahydronaphthyl, which, for the avoidance of doubt, is linked to the rest of the molecule through the aromatic ring.

Referring to general formula (I), a halogen atom may be a fluorine, chlorine, bromine or iodine atom.

Referring to the general formula (I), C₁₋₃perfluoroalkyl and C₁₋₃perfluoroalkoxy includes compounds which the hydrogens have been partially or fully replaced by fluorines, such as trifluoromethyl and trifluoromethoxy or trifluoroethyl. Referring to the general formula (I) reference to a C₃₋₈ cycloalkyl group means any single carbocyclic ring system, wherein said ring is fully or partially saturated. Suitable examples include cyclopropyl and cyclohexyl groups.

Referring to the general formula (I), reference to a heterocyclyl group means any single ring or fused ring system containing at least one ring heteroatom independently selected from O, N and S. Thus, a polycyclic fused ring system containing one or more carbocyclic fused saturated, partially unsaturated, or aromatic rings (usually benzo rings) is within the definition of heterocyclyl so long as the system also contains at least one fused ring which contains at least one of the aforementioned heteroatoms. As a substituent, such heterocyclyls may be attached to the remainder of the molecules from either a carbocyclic (e.g. benzo) ring or from a heterocyclic ring.

Referring to the general formula (I), a 5-6 membered hetroaromatic group includes a single aromatic ring system containing at least one ring heteroatom independently selected from O, N and S. Suitable examples include pyridyl and thiazolyl.

Referring to the general formula (I), a bicyclic heteroaromatic group includes a 5-6 membered heteroaromatic group fused to a phenyl or another heteroaromatic group, where each each heteroaromatic group contains at least one ring heteroatom independently selected from O, N and S. Suitable examples include benzothiophene, indole and benzofuran groups.

Referring to the general formula (I), reference to a group as containing one or more rings is intended to mean any single or fused cyclic moiety or moieties. The rings may be carbocyclic or heterocyclic, saturated or partially unsaturated, and aromatic or non-aromatic, as specified.

Reference to a polycyclic ring system or radical means that all rings in the system are fused.

Suitably, Ar₁ represents an optionally substituted phenyl, naphthyl, 1,2,3,4-tetrahydronaphthyl or bicyclic heteroaromatic group, e.g. indolyl or benzothiophenyl, where optional substitution is effected by R¹. More suitably, Ar₁ represents a substituted phenyl or naphthyl. Preferably Ar₁ represents a substituted phenyl. Equally preferably, Ar₁ represents a substituted naphthyl. Equally preferably, Ar₁ represents a substituted 1,2,3,4-tetrahydronaphthyl.

Substitution on Ar₁ is suitably represented by methylenedioxy or one, two or three groups independently selected from C₁₋₄ alkyl, e.g. methyl, ethyl or iso-propyl, hydroxy, C₁₋₄ alkoxy, e.g. methoxy or ethoxy, —O—C₀₋₄alkylene-R², e.g. —O-methylene-R², where R² represents C₁₋₄ perfluoroalkyl, e.g. trifluoromethyl, a 5-6 membered heteroaromatic group, e.g. pyridyl, preferably 2-pyridyl, or a C₃₋₈cycloalkyl, e.g. cyclopropyl.

Preferably, Ar₁ is a phenyl group substituted by methylenedioxy, preferably 3,4-methylenedioxy, or two or three groups independently selected from methyl, ethyl, isopropyl, hydroxy, methoxy, ethoxy, cyclopropylmethoxy and 2-pyridylmethoxy. Preferably, substitution is in two or three of the 2-, 4- or 5-positions on the phenyl ring. Most preferably, Ar₁ is a phenyl group substituted by 2-ethoxy and 4-methyl.

A is suitably —C(H)— or —N—, preferably —C(H)—.

Z is suitably a direct link, —NH—, —NSO₂Ph- or —O—. Z is preferably a direct link.

Integers o and n are preferably 1 and 2 respectively.

E is suitably an n-butylene or n-pentylene group. E is preferably an n-butylene group.

X is suitably a —N(H)CO— group, a —CON(H)— or —O— group. X is preferably an —N(H)CO— group.

G is suitably Y—Ar₃. Y is suitably an —N(H)CO— group, oxo, C₁₋₂alkylene, e.g. ethylene, C₂₋₃alkenylene, e.g. ethylene, —O—CH₂— or a direct link. Preferably, Y is an —N(H)CO— group. Equally preferably, Y is a direct link.

Where Ar₂ is a 5-6-membered heteroaromatic group, this is suitably a thiazolyl group, optionally substituted by C₁₋₄ alkyl, e.g. methyl. Where Ar₂ is a bicyclic heteroaromatic group, this is suitably a benzofuranyl or indolyl group, optionally substituted by C₁₋₄alkyl, e.g. methyl. Ar₂ is preferably phenyl and is suitably para-substituted.

Suitable electron withdrawing groups on Ar₃ include halogen, nitrile, nitro, C₁₋₄, C₁₋₄ perfluoroalkyl, C₁₋₄ acyl, C₁₋₄ alkoxycarbonyl, aminocarbonyl, C₁₋₄ alkylaminocarbonyl; di-C₁₋₄ alkylaminocarbonyl, C₁₋₄ alkylsulfonyl, C₁₋₄ alkylaminosulfonyl and di-C₁₋₄ alkylaminosulfonyl, C₁₋₄ alkylsulfonyl and C₁₋₄alkylsulfoxy.

Ar₃ is preferably phenyl or a pyridyl group, suitably 2-pyridyl, substituted by a halogen, e.g. chloro, nitrile or C₁₋₄perfluoroalkyl, e.g. trifluoromethyl. Most preferably, Ar₃ is phenyl substituted by chloro chloro, nitrile or trifluoromethyl.

When Ar₃ is phenyl, para-substitution is preferred.

A further aspect of the present invention is represented by the use of a compound of formula (Ia)

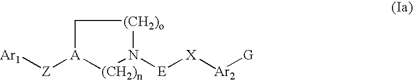

wherein

Ar₁ represents

(i) phenyl, naphthyl, or phenyl fused by a C₃₋₈cycloalkyl,

(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,

where Ar₁ optionally bears 1-4 groups independently represented by R¹;

R¹ is selected from halogen, —O—(C₀₋₄ alkylene)-R² or —(C₁₋₄alkylene)-R², where each alkylene group may additionally incorporate an oxygen in the chain, with the proviso that there are at least two carbon atoms between any chain heteroatoms;

R² represents

(i) hydrogen, C₁₋₄ perfluoroalkyl, C₁₋₄perfluoroalkoxy,

(ii) phenyl, phenyl fused by a C₃₋₈cycloalkyl, naphthyl or a 5- or 6-membered heteroaromatic group, optionally substituted by one or two groups independently selected from halogen, C₁₋₄ alkyl, hydroxy, C₁₋₄ alkoxy, amino, C₁₋₄ alkylamino and di-C₁₋₄ alkylamino,

(iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the Cm cycloalkyl or a monocyclic heterocyclyl may bear one or two groups independently selected from halogen, Cage alkyl, hydroxy, C₁₋₄ alkoxy, amino, C₁₋₄ alkylamino and di-C₁₋₄ alkylamino, or

(iv) amino, C₁₋₄ alkylamino or di-C₁₋₄alkylamino,

with the proviso that there are at least two carbon atoms between any chain heteroatoms;

Z is a direct link, oxo, —C(H)R³— or —SO₂—;

R³ is hydrogen, C₁₋₄ alkyl or phenyl, said phenyl optionally bearing one or two groups indpendently selected from halogen, C₁₋₄ alkyl, C₁₋₄ alkoxy and OH;

A is C—R⁴ or N;

n is an integer selected from 1-3;

o is an integer selected from 1-2;

R⁴ is hydrogen, C₁₋₄ alkyl, hydroxy or phenyl, said phenyl optionally bearing one or two groups indpendently selected from halogen, C₁₋₄ alkyl, C₁₋₄ alkoxy or hydroxy, or R⁴ forms a double bond between A and an adjacent ring carbon;

E is a C₁₋₆ alkylene group, optionally containing one or two double bonds or one triple bond;

X is a bond, —O—, oxo, —CON(H or C₁₋₄ alkyl)-, —N(H or C₁₋₄ alkyl)CO—, —N(H or C₁₋₄ alkyl)SO₂— or —SO₂N(H or C₁₋₄ alkyl)-;

Ar₂ is phenyl or a 5-14 membered heteroaromatic group, optionally substituted by one or two groups independently selected from C₁₋₄ alkyl, halogen, hydroxy, C₁₋₄ alkoxy, C₁₋₆ acyl, C₁₋₆ acyloxy, amino, C₁₋₄ alkylamino and di-C₁₋₄ alkylamino groups;

G is —Y—Ar₃;

Y is a bond, oxo, —O—, —N(H or C₁₋₄ alkyl)CO—, —CON(H or C₁₋₄ alkyl)-, —N(H or C₁₋₄ alkyl)SO₂—, or —SO₂N(H or C₁₋₄ alkyl)-, C₁₋₂ alkylene or C₂₋₃alkenylene;

Ar₃ represents

(iii) phenyl, naphthyl, or phenyl fused by a C₃₋₈cycloalkyl,

(iv) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,

where Ar₃ optionally bears 14 groups independently selected from halogen, nitrile, C₁₋₄ alkyl, C₁₋₄ alkoxy, C₂₋₄ alkenyl, C₂₋₄ alkenyloxy, hydroxy, azido, C₁₋₄perfluoroalkyl, C₁₋₄perfluoroalkoxy C₁₋₄ acyl, C₁₋₄ acyloxy, C₁₋₄ alkoxycarbonyl, aminocarbonyl, C₁₋₄ alkylaminocarbonyl; di-C₁₋₄ alkylaminocarbonyl, C₁₋₄ acylamino, amino, C₁₋₄ alkylamino or di-C₁₋₄ alkylamino groups;

or a physiologically acceptable salt, solvate or derivative thereof, in the manufacture of a medicament for the treatment of diseases ameliorated by LDL-r upregulation.

A yet further aspect of the present invention is represented by the use of a compound of formula (Ib)

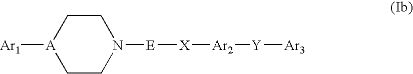

wherein

Ar₁ represents a phenyl, naphthyl or phenyl fused by a C₃₋₈cycloalkyl, where each group is optionally substituted by methylenedioxy or one to four groups independently represented by R¹;

Ar₂ represents a phenyl or 5-6 membered heteroaromatic group, optionally substituted by one to four groups independently selected from halogen, C₁₋₄ alkyl and C₁₋₄ alkoxy;

Ar₃ represents a phenyl or a 5-6 membered heteroaromatic group, optionally substituted by one to four groups independently selected from halogen, hydroxy, nitrile, C₁₋₄ alkyl, C₁₋₄ alkoxy, C₂₋₄ alkenyl, C₂₋₄ alkenyloxy, C₁₋₄ perfluoroalkyl, C₁₋₄ perfluoralkoxy, C₁₋₄ acyl, C₁₋₄ alkoxycarbonyl, aminocarbonyl, C₁₋₄ alkylaminocarbonyl; di-C₁₋₄ alkylaminocarbonyl and C₁₋₄ acylamino;

A represents —C(H or C₁₋₄ alkyl)- or —N—;

E represents —C₁₋₆ alkylene-;

X represents —CON(H or C₁₋₄alkyl)- or —N(H or C₁₋₄alkyl)CO—;

Y represents a direct link, N(H or C₁₋₄alkyl)CO— or —CON(H or C₁₋₄alkyl)-;

R¹ represents halogen, —O—(C₀₋₄ alkylene)-R² or —(C₀₋₄alkylene)-R², where each alkylene group may additionally incorporate an oxygen in the chain, with the proviso that there are at least two carbon atoms between any chain heteroatoms;

R² represents

(i) hydrogen; C₁₋₄ perfluoroalkyl, C₁₋₄ perfluoroalkoxy

(ii) phenyl, naphthyl, a 5- or 6-membered heteroaromatic group or 1,2,3,4-tetrahydronaphthyl, optionally substituted by one or two halogen, hydroxy, C₁₋₄ alkyl, 0-4 alkoxy groups,

(iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated or partially unsaturated,

(iv) amino, C₀₋₄ alkylamino or di-C₁₋₄alkylamino, with the proviso that there are at least two carbon atoms between any chain heteroatoms;

or a physiologically acceptable salt or solvate thereof, in the manufacture of a medicament for the treatment of diseases ameliorated by LDL-r upregulation.

The present invention also embraces novel compounds, which have been hereinbefore described. According to a further or alternative aspect of the present invention, there is provided a compound of formula (Ic)

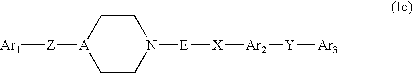

wherein

Ar₁ represents

(iii) phenyl, naphthyl, or phenyl fused by a C₃₋₈cycloalkyl,

(iv) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,

where Ar₁ optionally optionally bears 1-4 groups independently represented by R¹;

R¹ is selected from halogen, —S(C₁₋₄ alkyl), —O—(C₀₋₄ alkylene)-R² or C₀₋₄alkylene)-R², where each alkylene group may additionally incorporate an oxygen in the chain, with the proviso that there are at least two carbon atoms between any chain heteroatoms;

R² represents

(i) hydrogen, C₁₋₄ perfluoroalkyl, C₁₋₄perfluoroalkoxy,

(ii) phenyl, phenyl fused by a C₃₋₈cycloalkyl, naphthyl or a 5- or 6-membered heteroaromatic group, optionally substituted by one or two groups independently selected from halogen, C₁₋₄ alkyl, hydroxy, C₁₋₄ alkoxy, amino, C₁₋₄ alkylamino and di-C₁₋₄ alkylamino,

(iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-C₁₋₄ alkylamino, or

(iv) amino, C₁₋₄ alkylamino or di-C₁₋₄alkylamino,

with the proviso that there are at least two carbon atoms between any chain heteroatoms;

Z is a direct link, oxo, —O—, C(H)R³, —N(R⁵)—, —N(SO₂R⁶)— or —SO₂—;

R³ is hydrogen, C₁₋₄ alkyl or phenyl, said phenyl optionally bearing one or two groups indpendently selected from halogen, C₁₋₄ alkyl, C₁₋₄ alkoxy and OH;

A is C—R⁴ or N;

E represents a C₄₋₅alkylene group;

R⁴ is hydrogen, C₁₋₄ alkyl, hydroxy or phenyl, said phenyl optionally bearing one or two groups indpendently selected from halogen, C₁₋₄ alkyl, C₁₋₄ alkoxy or hydroxy, or R³ forms a double bond between A and an adjacent ring carbon;

R⁵ is C₁₋₄ alkyl or phenyl;

R⁶ is C₁₋₄ alkyl or phenyl;

X is a bond, —O—, oxo, —CON(H or C₁₋₄ alkyl)-, —N(H or C₁₋₄ alkyl)CO—, —N(H or C₁₋₄ alkyl)SO₂— or —SO₂N(H or C₁₋₄ alkyl)-;

Ar₂ is phenyl, a 5-6 membered heteroaromatic group or fused bicyclic aromatic radicals, wherein said radicals contain a total of from 8-12 ring atoms, wherein said radicals contain a total of from 1-4 ring heteroatoms independently selected from oxygen, nitrogen and sulfur, where each group is optionally substituted by one or two groups independently selected from C₁₋₄ alkyl, halogen, hydroxy, C₁₋₄ alkoxy, C₁₋₆ acyl, C₁₋₄ acyloxy, amino, C₁₋₄ alkylamino and di-C₁₋₄ alkylamino groups;

Y is a bond, oxo, —O—, —N(H or C₁₋₄ alkyl)CO—, —CON(H or C₁₋₄ alkyl)-, —N(H or C₁₋₄ alkyl)SO₂— or —SO₂N(H or C₁₋₄ alkyl)-, —C₁₋₂ alkylene-, —O—C₁₋₂ alkylene- or —C₂₋₃alkenylene-;

Ar₃ represents

(v) phenyl, naphthyl, or phenyl fused by a C₃₋₈cycloalkyl,

(vi) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,

where Ar₃ optionally bears 1-4 groups independently selected from halogen, nitrile, C₁₋₄ alkyl, C₁₋₄ alkoxy, C₂₋₄ alkenyl, C₂₋₄ alkenyloxy, hydroxy, azido, C₁₋₄perfluoroalkyl, C₁₋₄perfluoroalkoxy, nitro, C₁₋₄ alkylsulfonyl, C₁₋₄ alkylaminosulfonyl, C₁₋₄ dialkylaminosulfonyl, C₁₋₄ acyl, C₁₋₄ acyloxy, C₁₋₄ alkoxycarbonyl, aminocarbonyl, C₁₋₄ alkylaminocarbonyl, di-C₁₋₄ alkylaminocarbonyl, C₁₋₄ acylamino, amino, C₁₋₄ alkylamino or di-C₁₋₄ alkylamino groups;

or a physiologically acceptable salt, solvate or derivative thereof, with the proviso that compounds of formula (A) are excluded

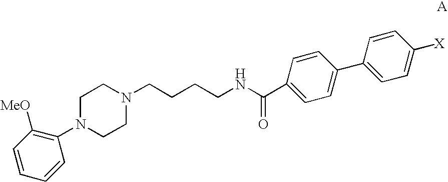

where X may be COMe, SO₂Me and NH₂. As a further aspect, the present invention provides a compound of formula (Ic) as described above with the additional proviso that 4-(4-chloro-benzoylamino)-N-{4-[4-(2-ethoxy-4-ethyl-phenyl)-piperidin-1-yl]-butyl}-benzamide and 4-(4-Benzoyl)-N-(4-[4-(4-isopropyl-2-methoxy-phenyl)-piperidin-1-yl]-butyl)benzamide are also excluded.

It will be understood that references herein to a compound of formula (I) apply equally to a compound of formula (Ia), (Ib) or (Ic) as appropriate.

Particularly preferred compounds of the invention include those in which each variable in Formula (I) is selected from the preferred groups for each variable. Even more preferable compounds of the invention include those where each variable in formula (I) is selected from the more preferred or most preferred groups for each variable.

Suitable compounds according to the invention include:

4-(4-chloro-benzoylamino)-N-{4-[4-(2,4-dimethoxy-phenyl)-piperidin-1-yl]-butyl}-benzamide;

4-(4-chloro-benzoylamino)-N-{4-[4-(2-ethoxy-4-methyl-phenyl)-piperidin-1-yl]butyl}-benzamide;

4-(4-chloro-benzoylamino)-N-{4-[4-(2-ethoxy-4-ethyl-phenyl)-piperidin-1-yl]-butyl}-benzamide;

4-(4-chloro-benzoylamino)-N-{4-[4-(4-ethyl-2-methoxy-phenyl)-piperidin-1-yl]-butyl}-benzamide;

4-(4-chloro-benzoylamino)-N-{4-[4-isopropyl-2-methoxy-phenyl)-piperidin-1-yl]-butyl}-benzamide;

4-(4-chloro-benzoylamino)-N-{4-[4-(2-ethoxy-4-isopropyl-phenyl)-piperidin-1-yl]-butyl}benzamide;

4′-Trifluoromethyl-biphenyl-4-carboxylic acid (4-{4-[2,5-dimethyl-4-(pyridin-2-ylmethoxy)-phenyl]-piperidin-1-yl}butyl)-amide;

4-(4-chloro-benzoylamino)-N-[4-(4-benzo[1,3]dioxol-5-yl-piperidin-1-yl]-butyl}-benzamide;

4-(4-chloro-benzoylamino)-N-[4-(4-naphthalen-2-yl-piperidin-1-yl]-butyl]-benzamide;

4-(4-chloro-benzoylamino)-N-{4-[4-(5,6,7,8-tetrahydro-naphthalen-2-yl)-piperidin-1-yl]-butyl}-benzamide;

4-(4-chloro-benzoylamino)-N-[4-(4-naphthalen-1-yl-piperidin-1-yl]-butyl}-benzamide;

4-(4-chloro-benzoylamino)-N-[4-(2-trifluoroethoxy-4-methyl-phenyl)-piperidin-1-yl]-butyl}-benzamide;

4′-Trifluoromethyl-biphenyl-4-carboxylic acid {4-[4-(2-methylsulfanyl-phenyl)piperidin-1-yl]-butyl}-amide;

4′-Trifluoromethyl-biphenyl-4-carboxylic acid {4-[4-(1-methyl-1H-indol-3-yl)-piperidin-1-yl]-butyl}-amide;

4′-Trifluoromethyl-biphenyl-4-carboxylic acid {4-[4-(1H-indol-3-yl)-piperidin-1-yl]butyl}-amide;

4′-Trifluoromethyl-biphenyl-4-carboxylic acid [4-(4-benzo[b]thiophen-3-yl-piperidin-1-yl)-butyl]-amide;

4-(4-chloro-benzoylamino)-N-{4-[4-(2,4-dimethoxy-phenyl)-piperazin-1-yl]-butyl}-benzamide;

4-(4-Chloro-benzoylamino)-N-{4-[4-(2,4-dimethoxy-phenyl)-[1,4]diazocan-1-yl]-butyl}-benzamide;

4′-Trifluoromethyl-biphenyl-4-carboxylic acid, {4-[4-(2-ethoxy-4-methyl-phenylamino)-piperidin-1-yl]-butyl}-amide;

4′-Trifluoromethyl-biphenyl-4-carboxylic acid (4-{4-[benzenesulfonyl-(2-ethoxy-4-methyl-phenyl)-amino]-piperidin-1-yl}-butyl)-amide;

4′-Trifluoromethyl-biphenyl-4-carboxylic acid {4-[4-(naphtalen-1-yloxy)-piperidin-1-yl]-butyl}-amide;

4-(4-chloro-benzoylamino)-N-{4-[4-(2-methoxy-4-methyl-phenyl)-piperazin-1-yl]-butyl}-benzamide;

4′-Trifluoromethyl-biphenyl-4-sulfonic acid {4-[4-(2-ethoxy-4-methyl-phenyl)-piperidin-1-yl]-butyl}-amide;

5-[4-(2-Ethoxy-4-methyl-phenyl)-piperidin-1-yl]-pentanoic acid (4′-trifluoromethyl-biphenyl-4-yl)-amide;

4′-{5-[4-(1-Methoxy-naphtalen-2-yl)-piperidin-1-yl]-pentyloxy}-biphenyl-4-carbonitrile;

4′-{4-[4-(1-Methoxy-naphtalen-2-yl)-piperidin-1-yl]-butoxy}biphenyl-4-carbonitrile;

4-Methyl-2-(4-trifluoromethyl-phenyl)-thiazole-5-carboxylic acid {4-[4-(4-isopropyl-2-methoxy-phenyl)-piperidin-1-yl]-butyl}-amide;

2-(4-Chloro-phenyl)-1-methyl-1H-indole-5-carboxylic acid {4-[4-(2-ethoxy-4-methyl-phenyl)-piperidin-1-yl]-butyl}-amide;

2-(4-Trifluoromethyl-phenyl)-benzofuran-5-carboxylic acid {4-[4-(2-ethoxy-4-methyl-phenyl)-piperidin-1-yl]-butyl}-amide;

2-(4-Chloro-phenyl)-benzofuran-5-carboxylic acid {4-[4-(2-ethoxy-4-methyl-phenyl)-piperidin-1-yl]-butyl}-amide;

2-(3,4-Dichloro-phenyl)-benzofuran-5-carboxylic acid {4-[4-(1-cyclopropylmethoxy-5,6,7,8-tetrahydro-naphtalen-2-yl)-piperidin-1-yl]-butyl}-amide;

2-(6-Trifluoromethyl-pyridin-3-yl)-benzofuran-5-carboxylic acid {4-[4-(1-cyclopropylmethoxy-5,6,7,8-tetrahydronaphtalen-2-yl)-piperidin-1-yl]-butyl}-amide;

N-{4-[4-(2-Ethoxy-4-methyl-phenyl)-piperidin-1-yl]-butyl}-4-[2-(4-trifluoromethylphenyl)-vinyl]-benzamide;

N-{4-[4-(2-Ethoxy-4-methyl-phenyl)-piperidin-1-yl]-butyl}-4-(4-trifluoromethyl-benzyloxy)-benzamide;

4-[2-(3,5-dichloro-phenyl)-ethenyl]-N-{4-[4-(2,4-dimethoxy-phenyl)-piperazin-1-yl]-butyl}-benzamide;

4-[2-(3,5-dichloro-phenyl)-ethyl]-N-{4-[4-(2,4-dimethoxy-phenyl)-piperazin-1-yl]butyl}-benzamide;

4-(4-Benzoyl)-N-{4-[4-(4-isopropyl-2-methoxy-phenyl)-piperidin-1-yl]-butyl}-benzamide;

4-trifluoromethyl-biphenyl-4-carboxylic acid {4-[4-(2,4-diethoxy-benzyl)-piperidin-1-yl]-butyl}-amide;

4-(4-chloro-benzoylamino)-N-{4-[4-(2,4-dimethoxy-benzoyl)-piperidin-1-yl]-butyl}-benzamide;

4′-Cyano-biphenyl-4-carboxylic acid {4-[4-(1−methyl-1H-indol-3-yl)-piperidin-1-yl]-butyl}-amide;

4-(4-chloro-benzoylamino)-N-{4-[4-(5-methyl-2-piperidin-4-yl-phenol)]-butyl}-benzamide;

4-(4-chloro-benzoylamino)-N-{4-[4-(5-ethyl-2-piperidin-4-yl-phenol)]-butyl}-benzamide;

4-(4-Chloro-benzoylamino)-N-{4-[4-(1−hydroxy-5,6,7,8-tetrahydro-naphtalen-2-yl)-piperidin-1-yl]-butyl}-benzamide;

4-(4-Chloro-benzoylamino)-N-{4-[4-(1-hydroxy-naphtalen-2-yl)-piperidin-1-yl]-butyl}-benzamide;

or a physiologically acceptable salt, solvate or derivative thereof.

The compounds of the invention are inducers of LDL-R expression and are thus of use in the treatment of conditions ameliorated by up-regulation of LDL-R expression.

The ability of the compounds of the invention to induce LDL-r expression by human hepatocytes in vitro is determined using a human hepatocarcinoma cell line, Hep G2, as a model system. A reporter gene assay using the LDL-R promotor in front of the Luciferase reporter gene is used as a primary screen.

The in vivo profile of the compounds is evaluated by oral administration of the compounds of the invention to fat-fed hamsters. Measurements of VLDL/LDL cholesterol and triglycerides upon treatment allow to determine the activity.

The compounds of the invention are potent and specific inducers of LDL-R expression, which furthermore exhibit good oral bioavailability and duration of action.

Compounds of the invention are of use in the treatment of diseases in which lipid imbalance is important, e.g. atherosclerosis, pancreatitis, non-insulin dependent diabetes mellitus (NIDDM), coronary heart diseases and obesity.

Compounds of the invention are also useful in lowering serum lipid levels, cholesterol and/or triglycerides, and are of use in the treatment of hyperlipemia, hyperlipidemia, hyperlipoproteinemia, hypercholesterolemia and/or hypertriglyceridemia.

The invention therefore provides a compound of formula (Ic) or a physiologically acceptable salt, solvate or derivative thereof for use in therapy, in particular in human medicine.

In an alternative or further aspect there is provided a method for the treatment of a mammal, including man, in particular in the treatment of diseases ameliorated by LDL-R up-regulation, comprising administration of an effective amount of a compound of formula (I) or a physiologically acceptable salt, solvate or derivative thereof.

There is also provided as a further aspect of the invention the use of a compound of formula (Ic) or a physiologically acceptable salt, solvate or derivative thereof in the preparation of a medicament for use in the treatment of diseases ameliorated by LDL-R up-regulation.

It will be appreciated that reference to treatment is intended to include prophylaxis as well as the alleviation of established symptoms. Compounds of formula (I) may be administered as the raw chemical but the active ingredient is preferably presented as a pharmaceutical formulation.

Accordingly, the invention also provides a pharmaceutical composition which comprises at least one compound of formula (I) or a physiologically acceptable salt, solvate or derivative thereof and formulated for administration by any convenient route. Such compositions are preferably in a form adapted for use in medicine, in particular human medicine, and can conveniently be formulated in a conventional manner using one or more pharmaceutically acceptable carriers or excipients.

Thus compounds of formula (I) may be formulated for oral, buccal, parenteral, transdermal, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or lozenges formulated in conventional manner.

For transdermal administration the compounds according to the invention may be formulated as creams, gels, ointments or lotions or as a transdermal patch. Such compositions may for example be formulated with an aqueous or oily base with the addition of suitable thickening, gelling, emulsifying, stabilising, dispersing, suspending, and/or colouring agents.

The compounds of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unit dose device or alternatively as a powder mix with a suitable carrier for administration using a suitable delivery device.

The compositions may contain from 0.1% upwards, e.g. 0.1-99% of the active material, depending on the method of administration. A proposed dose of the compounds of the invention is 0.25 mg/kg to about 125 mg/kg bodyweight per day e.g. 20 mg/kg to 100 mg/kg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

The compounds of formula (I) may, if desired, be administered with one or more therapeutic agents and formulated for administration by any convenient route in a conventional manner. Appropriate doses will be readily appreciated by those skilled in the art. For example, the compounds of formula (I) may be administered in combination with any agent which raises HDL, an HMG CoA reductase inhibitor, an agent for inhibition of bile acid transport or a fibrate.

A compound of formula (I) or (Ia), or a physiologically acceptable salt, solvate or derivative thereof, may be prepared by the general methods outlined hereafter. In the following description, the groups Ar₁, Z, A, E, X, Ar₂, G, n and o are as previously defined for compounds of formula (Ia), unless specified otherwise.

According to a first general process (A), a compound of formula (I) may be prepared by reaction of a compound of formula (II) with a compound of formula (III)

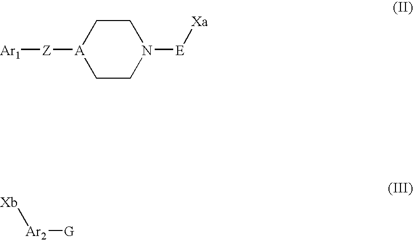

where Xa and Xb are suitable reactants to form a group X. For example, where X is N(H or C₁₋₄ alkyl)CO, Xa is NH₂ or NH(C₁₋₄ alkyl) and Xb is COL where L is OH or a suitable leaving group, such as halide. Such a reaction may be effected under standard amide bond-forming conditions, including those described herein.

A compound of formula (II) where Xa is NH₂ or NH(C₁₋₄ alkyl), ray be prepared by reaction of a compound of formula (IV) with a compound of formula (V)

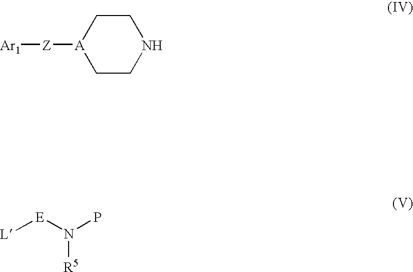

where R⁵ represents H or C₁₋₄alkyl, L′ is a suitable group, such as halide, and P is any suitable N-protecting group, under standard alkylation conditions, including those described herein, followed by removal of the protecting group under standard conditions.

A compound of formula (II) where Xa is NH₂ or NH(C₁₋₄ alkyl), may further be prepared by reaction of a compound of formula (IV) with a compound of formula (Va)

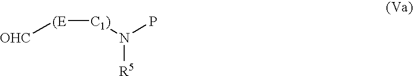

where R⁵ represents H or C₁₋₄alkyl, E-C₁ (E minus C₁) represents the group E with one less carbon group in its chain and P is any suitable N-protecting group, under standard reductive amination conditions, including those described herein, followed by removal of the protecting group under standard conditions.

A compound of formula (IV), where A is CH, may be prepared by reaction of a compound Ar₁-sal, where sal represents the lithium or magnesium ion of Ar₁, with a compound of formula (VI)

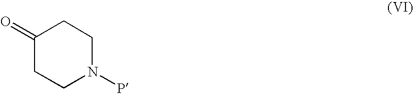

where P′ represents a suitable N-protecting group, such as acetyl, benzyl or benzyl-4-oxo-1 carboxylate, followed by the steps of dehydration, reduction of the resulting double bond, and finally, removal of the protecting group P′. Such chemistry has been described, for example, in European Patent Appliction no. 0630887.

Alternatively, a compound of formula (IV) where A is CH and Ar₁ is substituted by an activated ortho or para activating group for the reaction centre, Act, e.g. methoxy or hydroxy and A is CH, may be prepared by reaction of a compound of formula Ar₁-Act, with a compound of formula (VI) under suitable reaction conditions such as e.g. trifluoroborane or acetic acid and aqueous hydrochloric acid, to form a tetrahydropyridyl ring, followed by reduction, e.g. under hydrogenation conditions, of the resulting double bond and finally deprotection of the N-protecting group, P′ under standard conditions.

Alternatively, a compound of formula (IV) where where A is CH and Ar₁ is substituted by an activated ortho or para activating group for the reaction centre, Act, e.g. methoxy or hydroxy and A is CH, may be prepared by reaction of a compound of formula Ar₁-Act, with a compound of formula (VII)

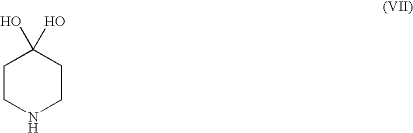

under suitable reaction conditions such as e.g. acetic acid and aqueous hydrochloric acid to form a tetrahydropyrdyl ring, followed by suitable N-protection, then reduction, e.g. under hydrogenation conditions, of the resulting double bond and finally deprotection of the N-protecting group.

Alternatively, a compound of formula (IV), where A is N and Z is a direct rink, may be prepared by reaction of a compound of formula (VIII) with a compound of formula (IX)

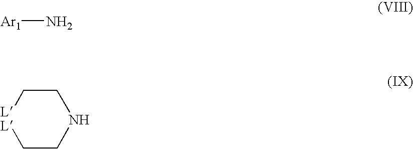

where the group L′ is a suitable leaving group, such as a halide, e.g. chloride, under suitable conditions for alkylation, such as with a base such as sodium carbonate in solvent such as n-butanol.

A compound of formula (III) may be prepared by standard methods including, where Xb is CO₂H, deprotection of a compound of formula (X)

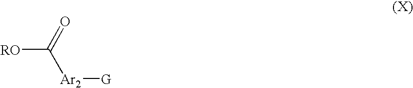

where R is a suitable carboxylic acid protecting group, such as methyl.

A compound of formula (X) where R is H or a suitable protecting group and G is Ar₃, may be prepared by reaction of a compound of formula (XI), with a compound of formula (XII)

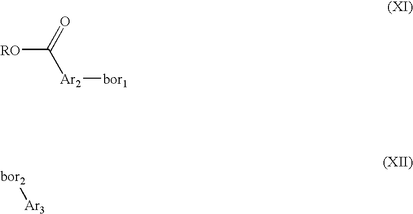

where bor₁ represents a boronic acid group or a halide, e.g. bromide or iodide, and bor₂ represents a suitable boronic acid group or a halide, e.g. bromide or iodide for coupling, under conditions suitable for boronic acid coupling, e.g. using palladium (0) and sodium carbonate.

According to a second general process (B), a compound of formula (I) may be prepared by reaction of a compound of formula (IV) with a compound of formula (XIII)

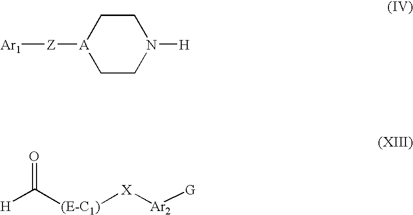

where E-C₁ (‘E minus C₁’) means that the chain length of group E is one carbon less than that in the resulting compound (I), under standard reductive amination conditions, e.g. sodium triacetoxyborohydride and acetic acid in a suitable solvent, such as dichloromethane.

A compound of formula (XIII) may be prepared by reaction of a compound of formula (XIV) with a compound of formula (XV)

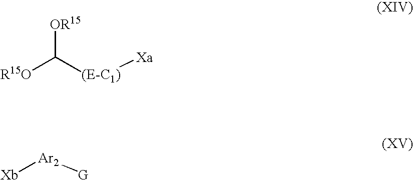

where R¹⁵ is a suitable alkyl protecting group for oxygen, such as methyl, and Xa and Xb are suitable reactants to form a group X, as defined hereinbefore, followed by removal of the protecting group, under acidic conditions.

According to a third general process (C), a compound of formula (I) may be prepared by reaction of a different compound of formula (I), by well known methods. For example a compound of formula (I) where Ar₁ is substituted by C₁₋₄ alkoxy may be prepared from the corresponding compound of formula (I) where the substituent is hydroxy by standard O-alkylation methods.

Compounds of formula (V), (VI), (VII), (VIII), (IX), (XI), (XIV) and (XV), are known or may be prepared by standard methods, e.g. as substantially described herein.

The protecting groups used in the preparation of compounds of formula (I) may be used in conventional manner. See for example ‘Protective Groups in Organic Chemistry’ Ed. J. F. W. McOmie (Plenum Press 1973) or ‘Protective Groups in Organic Synthesis’ by Theodora W Greene and P M G Wuts (John Wiley and Sons 1991).

Conventional amino protecting groups may include for example aralkyl groups, such as benzyl, diphenylmethyl or triphenylmethyl groups; and acyl groups such as N-benzyloxycarbonyl or t-butoxycarbonyl.

Conventional carboxylic acid protecting groups include methyl and ethyl groups.

The invention is further described with reference to the following non-limiting examples.

Abbreviations:

THF—Tetrahydrofuran, BF₃-Et₂O—Boron trifluoride diethyl etherate, DCM—Dichloromethane, TEA—triethylamine, EtOH— Ethanol, EtOAc—Ethyl acetate, IPr₂O—Di-isopropyl ether, TFA—Trifluoroacetic acid, Pd/C—Palladium on carbon, Et₂O—diethyl ether, IPrOH—Isopropanol, IprNH₂—Isopropylamine, Chex—cyclohexane, MeOH—Methanol, DMF—Dimethyl formamide, EDCl—1-(3-dimethylaminopropyl)-, ethylcarbodiimide hydrochloride, HOBt—1-Hydroxybenzotriazole, MeCN—Acetonitrile, rt—Room temperature, CDI—Carbonyle diimidazole, nBuOH—nButanol, AcOH—Acetic acid CH₃SO₃H—Methane sulfonic acid, MgSO₄—Magnesium sulfate, Na₂SO₄—Sodium sulfate, HATU—O-(7-Azabenzotriazol-1-yl)-N,N,N′N′-hetramethyluroniumhexafluorophosphate

Intermediate 1

4-(4-Chloro-benzoylamino)-benzoic Acid Ethyl Ester

A solution of 4-Amino-benzoic acid ethyl ester (124.0 g, 0.75 mol) in THF/DCM (500 mL/1000 mL) was treated with TEA (120 mL, 1.15 eq.) and 4-Dimethylaminopyridine (1.3 g, catalytic amount). At −7° C. a solution of 4-Chloro-benzoyl chloride (152 g, 1.15 eq.) in THF (100 mL) was added dropwise. The resulting mixture was stirred mechanically for 48 hours. The solvent was evaporated off and the residue was taken up in EtOAc/DCM (30/70). A concentrated NaOH solution was added until pH=12. A white solid precipated out and was collected (156.8 g, 0.52 mol). The organic layer was dried over Na₂SO₄. The solvent was evaporated off and crystallization from IPr₂O gave a second batch of the title compound (63.2 g, 0.21 mol).

¹H NMR (CDCl₃, 250 MHz) δ 8.1 (s, 1H), 7.9 (d, 2H), 7.7 (d, 2H), 7.6 (d, 2H), 7.3 (d, 2H), 4.3 (q, 2H), 1.3 (t, 3H).

Intermediate 2

4-(4-Chloro-benzoylamino)-benzoic Acid

A suspension of intermediate 1 (220 g, 0.72 mol) in 2000 mL of EtOH was treated with a 1N NaOH solution (1000 mL). The resulting suspension was heated at reflux overnight. A white solid precipated out. At reflux, concentrated HCl solution was added until pH=1. Under rigorous mechanical stirring, the resulting suspension was cooled down. A white solid was collected and dried under reduced pressure to give the title compound in a quantitative yield.

¹H NMR (DMSO d⁶, 250 MHz) δ 10.5 (s, 1H), 7.9 (d, 2H), 7.8 (s, 4H), 7.5 (d, 2H).

Ref: J. Pharm. Sci. (1979), 68(3), 332-5

Intermediate 3

4-(2,4-Dimethoxy-phenyl)-4-hydroxy-piperidine-1-carboxylic Acid Benzyl Ester

A solution of 1-Bromo-2,4-dimethoxy-benzene (16.0 g, 0.074 mol) in THF (200 mL) was cooled to −78° C. and treated with nBuLi (2.0 M in hexane, 37.0 mL, 1 eq.). The resulting mixture was stirred for one hour at −10° C. At −78° C. a solution of Benzyl-4-oxo-1-piperidine carboxylate (17.3 g, 1 eq.) in THF (15 mL) was added. The resulting mixture was allowed to stir at −78° C. for one hour and allowed to warm up to rt for 2 hrs. Addition of water (40 mL), extraction with EtOAc, drying over MgSO₄ and evaporation under reduced pressure gave a residue that was flash chromatographed using iPrOH/chex (10/90) as eluent. The title compound (21.48 g, 58,0 mmol) was isolated as a yellow oil in a 78% yield.

¹H NMR (CDCl₃, 250 MHz) δ 7.4 (m, 5H), 7.15 (d, 1H), 6.6 (d, 1H), 6.5 (dd, 1H), 5.2 (s, 2H), 4.2 (s, 1H), 3.9 (s, 3H), 3.8 (s, 3H), 3.5 (bt, 2H), 2.5 (t, 2H), 2.0 (m, 4H).

Intermediate 4

4-(2,4-Dimethoxy-phenyl)-piperidine-1-carboxylic Acid Benzyl Ester

A solution of intermediate 3 (1.5 g, 4.0 mmol) in DCM (40 mL) was treated with TFA (3 mL, 10 eq.) and triethyl silane (13 mL, 20 eq.) at rt. The resulting solution was allowed to stir at rt for 16 hours. The solvent was evaporated under reduced pressure. The residue was filtered through a bed of silica to give the tilte compound (1.4 g, 4.0 mmol) as a gummy beige solid in a 100% yield.

¹H NMR (CDCl₃, 250 MHz) δ 7.3 (m, 5H), 6.9 (d, 1H), 6.4 (m, 2H), 5.1 (s, 2H), 4.2 (m, 2H), 3.7 (s, 6H), 2.7 (m, 2H), 1.5-1.7 (m, 4H).

Intermediate 5

4-(2,4-Dimethoxy-phenyl)-piperidine

A solution of intermediate 4 (1.4 g, 4.0 mmol) in THF (40 mL) was treated with Pd/C 10%, (20%) under an atmospheric pressure of hydrogen. The resulting solution was allowed to stir at 40° C. for 16 hours. The reaction mixture was filtered through a bed of celite. The filtrate was evaporated under reduced pressure to give a the title compound (0.9 g, 4.0 mmol) with a quantitative yield.

GC/IMS: M+ C₁₃H₁₉NO₂ 221

¹H NMR (CDCl₃, 250 MHz) δ 7.0 (d, 1H), 6.4 (m, 2H), 3.7 (s, 6H), 3.5 (s, 1H), 3.2 (d, 2H), 3.0 (m, 1H), 2.7 (m, 2H), 1.5-1.7 (m, 4H).

Intermediate 6

2-{4-[4-(2,4-Dimethoxy-phenyl)-piperidin-1-yl]-butyl}-isoindole-1,3-dione

A solution of intermediate 5 (0.9 g, 4.1 mmol) in acetone (20 mL) was treated with Cs₂CO₃ (1.47 g, 1.1 equiv.) and N-(4-Bromobutyl)-phtalimide (1.27 g, 1.1 eq.). The resulting mixture was stirred at reflux for 16 hours. After cooling to rt the reaction mixture was filtered off. The cake was washed with acetone. The filtrate was evaporated off to give the title compound (1.26 g, 2.98 mmol) as a yellow oil in a 73% yield.

¹H NMR (CDCl₃, 250 MHz) δ 7.9 (m, 2H), 7.8 (m, 2H), 7.15 (d, 1H), 6.5 (m, 2H), 3.8 (d, 6H), 3.7 (m, 4H), 3.2 (bd, 2H), 2.9 (m, 1H), 2.6 (t, 2H), 2.2 (m, 2H), 2.0-1.6 (m, 6H).

Intermediate 7

4-[4-(2,4-Dimethoxy-phenyl)-piperidin-1-yl]-butylamine

A solution of intermediate 6 (1.26 g, 2.98 mmol) in MeOH (30 mL) was treated with hydrazine (0.6 mL). The resulting mixture was stirred at 60° C. for 16 hours. After cooling to rt, a 1N HCl solution was added until pH=4. After evaporation under reduced pressure the residue was taken up in water. Filtration gave a yellow solution that was treated with an aqueous solution of K₂CO₃. Extraction with DCM/MeOH (90/10), drying over Na₂SO₄ and filtration gave the title compound (0.43 g, 1.47 mmol) as a yellow oil in a 49% yield.

GC/MS: M+ C₁₇H₂₈BN₂O₂ 292

¹H NMR (CDCl₃, 250 MHz) δ 7.15 (d, 1H), 6.4 (m, 2H), 3.7 (d, 6H), 3.0 (bd, 2H), 2.8 (m, 1H), 2.6 (t, 2H), 2.4 (t, 2H), 2.0 (td, 2H), 1.7-1.4 (m, 8H).

Intermediate 8

1-[4-(2-Hydroxy-4-methyl-phenyl)-3,6-dihydro-2H-pyridin-1-yl]-ethanone

To a solution of m-Cresol (50.0 g, 0.46 mol) and 1-Acetyl-4-piperidone (65.4 g, 1.0 eq.) was added dropwise BF₃-Et₂O (176 mL, 3.0 eq). The mixture was stirred at 100° C. for 2 hours. After cooling to rt, the mixture was treated with a 1N HCl solution (800 mL). The resulting solution was extracted with DCM. The organic layer was dried over Na₂SO₄ and evaporated to dryness to give an oil which was crytallized in MeCN to give the title compound (60.0 g, 0.26 mol) as a white powder in a 57%.

GC/MS: M+ C₁₄H₁₇NO₂ 231

Intermediate 9

1-[4-(2-Hydroxy-4-methyl-phenyl)-piperidin-1-yl]-ethanone

To a solution of intermediate 8 (60.0 g, 0.26 mol) in EtOH (600 mL) and DCM (200 mL) was added Pd/C, 10% (6 g) and the reaction was stirred under an atmospheric pressure of hydrogen at rt for 48 hours. The reaction mixture was filtered through a bed of celite. The filtrate was evaporated under reduced pressure to give the title compound (55.0, 0.24 mol) as a white powder.

GC/MS: M+ C₁₄H₁₉NO₂ 233

Intermediate 10

1-[4-(2-Ethoxy-4-methyl-phenyl)-piperidin-1-yl]-ethanone

To a solution of intermediate 9 (55.0 g, 0.24 mol) in dry acetone (800 mL) was added anhydrous Cs₂CO₃ (93.0 g, 1.2 eq.) and ethyl iodide (23 mL, 1.2 eq.). The reaction was stirred under reflux for 18 hours. After cooling, the reaction was filtered off and washed with acetone. The filtrate was evaporated under reduced pressure to give the title compound as an oil (53.0 g, 0.20 mol).

GC/IMS: M+ C₁₆H₂₃NO₂ 261

Intermediate 11

4-(2-Ethoxy-4-methyl-phenyl)-piperidine

To a solution of intermediate 10 (53.0 g, 0.20 mol) in MeOH (600 mL) was added a solution of NaOH (260 mL) in H₂O (260 mL). The reaction was stirred under reflux for 48 hours. After cooling, the reaction was concentrated under reduced pressure, was diluted with DCM and washed with water. The organic layer was dried over Na₂SO₄ and evaporated to dryness to give the title compound (40.0 g, 0.18 mol) as a yellow oil.

GC/MS: M+ C₁₄H₂₁NO 219

Intermediate 12

4-(4-chloro-benzoylamino)-N-[4-(4,4-diethoxy-butyl)]-benzamide

To a solution of intermediate 2 (10.0 g, 36.3 mmol) in DMF (60 mL), was added 4-Aminobutyraldehyde diethyl acetal (6.44 g, 1.1 eq.), HOBt (7.35 g, 1.5 eq.), CDI (8.8 g, 1.5 eq.) and TEA (7.5 mL, 1.5 eq.). The reaction was stirred at rt for 24 hours. A precipitate was formed. Water (50 mL) was added and the reaction was filtered off. The precipitate was-washed with H₂O and dried to give the title compound (11.0 g, 26 mmol) as a white solid.

¹H NMR (DMSO, 250 MHz) δ 10.6 (s, 1H), 8.45 (t, 1H), 8.1 (d, 2H), 7.9 (s, 4H), 7.7 (d, 2H), 4.55 (m, 1H), 3.7-3.3 (m, 6H), 1.7 (m, 4H), 1.15 (t, 6H).

Intermediate 13

4-(4-chloro-benzoylamino)-N-[4-(4-oxo-butyl)]-benzamide

To a suspension of intermediate 12 (11.0 g, 26 mmol) in acetone (100 mL) was added a 1N HCl solution (50 mL). The reaction was stirred at reflux for 2 hours. The solvent was evaporated off and the aqueous phase was treated with a saturated NaHCO₃ solution until PH=9-11. The precipitate was filtered off, washed with H₂O and dried to give the title compound (8.3 g, 24 mmol) as a white powder.

MP: 220° C.

Intermediate 14

5-Ethyl-2-(1,2,3,6-tetrahydro-pyridin-4-yl)-phenol

A solution of 3-ethyl-phenol (122.2 g, 1 mol) and 4-piperidone hydrate hydrochloride (184.2 g, 1.2 eq) in acetic acid (500 mL) was treated with HCl gaz for 10 min. The mixture was stirred at 95° C. for 30 min. After cooling to room temperature, th mixture was treated again with HCl gaz for 5 min. The resulting solution was allowed to stir at room temperature for 4 days. The solvent was evaporated under reduced pressure to give the an colorless oil (200 g). The product was used without further purification.

Intermediate 15

Acetic acid 2-(1-acetyl-1,2,3,6-tetrahydro-pyridin-4-yl)-5-ethyl-phenyl Ester

To a solution of intermediate 14 (33 g, 0.162 mol) in pyridine (300 mL) was added acetic anhydride (100 mL). The mixture was stirred at room temperature for 4 hours. The solvents were evaporated under reduce pressure. The oil was diluted with DCM and washed with water. The organic layer was dried over Na₂SO₄ and evaporated to dryness to give the title compound (28 g, 0.097 mol) as a yellow oil in a 60% yield.

¹H NMR (CDCl₃, 250 MHz) δ 7 (m, 2H), 6.7 (m, 1H), 5.65 (m, 1H), 4.05 (m, 2H), 3.55 (dt, 2H), 2.6 (q, 2H), 2.3 (m, 2H), 2.15 (s, 3H), 2.05 (d, 3H), 1.1 (t, 3H).

Intermediate 16

1-[4-(4-Ethyl-2-hydroxy-phenyl)-3,6-dihydro-2H-pyridin-1-yl]-ethanone

To a solution of intermediate 15 (28 g, 0.098 mol) in methanol (700 mL) was added K₂CO₃ (40 g, 3 eq) and the mixture was stirred under reflux for 4 hours. The solution was filtered and the methanol was evaporated. The oil was diluted with DCM and washed with water. The organic layer was dried over Na₂SO₄ and evaporated to dryness to give the title compound (20 g, 0.082 mol) as a orange oil in a 84% yield.

¹H NMR (CDCl₃, 250 MHz) δ 6.7 (m, 2H), 6.6 (m, 1H), 5.8 (m, 1H), 4.1 (m, 2H), 3.65 (m, 2H), 2.7 (m, 5H), 2.4 (q, 2H), 1.2 (t, 3H).

Intermediate 17

1-[4-(4-Ethyl-2-hydroxy-phenyl)-piperidin-1-yl]-ethanone

To a solution of intermediate 16 (20 g, 0.082 mol) in methanol (600 mL) was added Pd/C, 10% (1.2 g) and the reaction was stirred under under an atmospheric pressure of hydrogen for 24 hours. The reaction mixture was filtered through a bed of celite. The filtrate was evaporated under reduced pressure to give the title compound (15 g, 0.06 mol) as an oil in a 75% yield.

¹H NMR (CDCl₃, 250 MHz) δ 6.85 (d, 1H), 6.6 (m, 2H), 4.65 (m, 1H), 3.8 (m, 1H), 3.2-2.9 (m, 2H), 2.6 (m, 1H), 2.45 (q, 2H), 2.05 (s, 3H), 1.7 (m, 2H), 1.5 (m, 2H), 1.1 (t, 3H).

Intermediate 18

1-[4-(2-Ethoxy-4-ethyl-phenyl)-piperidin-1-yl]-ethanone

To a solution of intermediate 17 (7.41 g, 0.03 mol) in dry acetone (150 mL) was added anhydrous Cs₂CO₃ (14.7 g, 1.5 eq) and ethyl iodide (4.8 mL, 2 eq). The reaction was stirred under reflux for 5 hours. After cooling, the reaction was filtered off and washed with acetone. The filtrate was evaporated under reduced pressure to give the title compound as an oil (8.2 g, 0.03 mol) in a quantitative yield.

¹H NMR (CDCl₃, 250 MHz) δ 6.9 (d, 1H), 6.6 (m, 2H), 4.7 (m, 1H), 4.0 (q, 2H), 3.8 (m, 1H), 3.1 (m, 2H), 2.5 (m, 3H), 2.05 (s, 3H), 1.7(m, 2H), 1.50 (m, 2H), 1.35 (t, 3H), 1.1 (t, 3H).

Intermediate 19

4-(2-Ethoxy-4-ethyl-phenyl)-piperidine

To a solution of intermediate 18 (8.17 g, 0.03 mol) in methanol (150 mL) was added a solution of NaOH (37 mL) in H₂O (37 mL). The reaction was stirred under reflux for 16 hours. After cooling, the reaction was concentrated under reduced pressure, was diluted with DCM and washed with water. The organic layer was dried over Na₂SO₄ and evaporated to dryness to give the title compound (6.6 g, 0.028 mol) as a yellow oil in a 94% yield.

¹H NMR (CDCl₃, 250-MHz) δ 7.1 (d, 1H), 6.7 (d, 1H), 4.7 (d, 1H), 4.05 (q, 2H), 3.1 (m, 2H), 3.05 (m, 1H), 2.7 (td, 2H), 2.55 (q, 2H), 1.75 (m, 3H), 1.55 (m, 2H), 1.35 (t, 3H), 1.1 (t, 3H).

Intermediate 20

2-{4-[4-(2-Ethoxy-4-ethyl-phenyl)-piperidin-1-yl]-butyl}-isoindole-1,3-dione

The same method was employed as in the preparation of Intermediate 6 but starting from Intermediate 19 gave the title compound as a yellow oil in a 97°/yield.

¹H NMR (CDCl₃, 250 MHz) δ 7.8 (m, 2H), 7.6 (m, 2H), 7.0 (d, 1H), 6.65 (dd, 1H), 6.55 (sd, 1H), 3.95 (q, 2H), 3.65 (m, 3H), 2.95 (m, 2H), 2.8 (m, 1H), 2.5 (q, 2H), 2.4 (m, 2H), 2 (td, 2H), 1.8-1.4 (m, 8H), 1.3 (t, 3H), 1.15 (t, 3H).

Intermediate 21

4-[4-(2-Ethoxy-4-ethyl-phenyl)-piperidin-1-yl]-butylamine

The same method was employed as in the preparation of Intermediate 7 but starting from Intermediate 20 gave the title compound as a yellow oil in a 81.5% yield.

¹H NMR (CDCl₃, 250 MHz) δ 7.1 (d, 1H), 6.7 (dd, 1H), 6.6 (s, 1H), 4.0 (q, 2H), 3.0 (bd, 2H), 2.9 (m, 1H), 2.7 (t, 2H), 2.55 (q, 2H), 2.3 (m, 2H), 2.0 (td, 2H), 1.71.2 (m, 10H), 1.4 (t, 3H), 1.1 (t, 3H).

Intermediate 22

1-[4-(4-Ethyl-2-methoxy-phenyl)-piperidin-1-yl]-ethanone

The same method was employed as in the preparation of intermediate 18, starting from intermediate 17, in using methyl iodide as alkylating reagent (6 eq), gave the title compound as an oil in a 94% yield.

GC/MS: M+ C₁₆H₂₃NO₂ 261

Intermediate 23

4-(4-Ethyl-2-methoxy-phenyl)-piperidine

The same method was employed as in the preparation of intermediate 19 but starting from intermediate 22 gave the title compound as an oil in a 92% yield.

GC/MS: M+ C₁₄H₂₁NO 219

Ref: Ger. Offen., 66 pp. DE 2801195

Intermediate 24

2-{4-[4-(4-Ethyl-2-methoxy-phenyl)-piperidin-1-yl]-butyl}-isoindole-1,3-dione

The same method was employed as in the preparation of Intermediate 6 but starting from Intermediate 23 gave the title compound as a yellow oil in a 71% yield.

¹H NMR (CDCl₃, 250 MHz) δ 7.8 (m, 2H), 7.65 (m, 2H), 7.05 (d, 1H), 6.7 (bd, 1H), 6.65 (bs, 1H), 3.7 (s, 3H), 3.65 (t, 2H), 3.0 (m, 2H), 2.9 (m, 1H), 2.6 (q, 2H), 2.4 (m, 2H), 2 (m, 2H), 1.8-1.5 (m, 8H), 1.2 (t, 3H).

Intermediate 25

4-[4-(4-Ethyl-2-methoxy-phenyl)-piperidin-1-yl]-butylamine

The same method was employed as in the preparation of Intermediate 7 but starting from Intermediate 24 gave the title compound as a oil in a 90% yield.

¹H NMR (CDCl₃, 250 MHz) δ 7.1 (d, 1H), 6.7 (m, 1H), 6.6 (s, 1H), 3.8 (t, 3H), 3.0 (bd, 2H), 2.9 (m, 1H), 2.7 (t, 2H), 2.6 (q, 2H), 2.35 (m, 2H), 2.05 (m, 2H), 1.8-1.4 (m, 10H), 1.25 (t, 3H).

Intermediate 26

15-Isopropyl-2-(1,2,3,6-tetrahydro-pyridin-4-yl)-phenol

A solution of 3-isopropyl-phenol (68.1 g 0.5 mol) and 4-piperidone hydrate hydrochloride (92.1 g, 1.2 eq) in acetic acid (300 mL) was treated with HCl gaz for 10 min. The mixture was stirred at 95° C. for 30 min. After cooling to room temperature, the mixture was treated again with HCl gaz for 5 min. The resulting solution was allowed to stir at room temperature for 4 days. The solvent was evaporated under reduced pressure to give the an colorless oil (110 g). The product was used without further purification.

Intermediate 27

Acetic Acid 2-(0.1-acetyl-1,2,3,6-tetrahydro-pyridin-4-yl)-5-isopropyl-phenyl Ester

To a solution of intermediate 26 (110 g, 0.5 mol) in pyridine (1000 mL) was added acetic anhydride (300 mL). The mixture was stirred at room temperature for 4 hours. The solvents were evaporated under reduce pressure. The oil was diluted with DCM and washed with water. The organic layer was dried over Na₂SO₄ and evaporated to dryness to give the title compound (150 g, 0.5 mol) as a yellow oil in a quantitative yield.

GC/MS: M+ C₁₈H₂₃NO₃ 301

Intermediate 28

1-[4-(2-Hydroxy-4-isopropyl-phenyl)-3,6-dihydro-2H-pyridin-1-yl]-ethanone

To a solution of intermediate 27 (150 g, 0.098 mol) in methanol (700 mL) was added K₂CO₃ (40 g, 3 eq) and the mixture was stirred under reflux for 4 hours. The solution was filtered and the methanol was evaporated. The oil was diluted with dichloromethane and washed with water. The organic layer was dried over Na₂SO₄ and evaporated to dryness to give the title compound (76 g, 0.29 mol) as a orange oil in a 59% yield.

GC/MS: M+ CO₁₆H₂₁NO₂ 259

Intermediate 29

1-[4-(2-Hydroxy-4-isopropyl-phenyl)-piperidin-1-yl]-ethanone

To a solution of intermediate 28 (56 g, 0.22 mol) in ethanol (1400 mL) was added Pd/C, 10% (5.6 g) and the reaction was stirred under under an atmospheric pressure of hydrogen for 24 hours. The reaction mixture was filtered through a bed of celite. The filtrate was evaporated under reduced pressure to give the title compound (54.5 g, 0.21 mol) as an oil in a quantitative yield.

GC/MS: M+ C₁₅H₂₃NO₂ 261

Intermediate 30

1-[4-(4-Isopropyl-2-methoxy-phenyl)-piperidin-1-yl]-ethanone

To a solution of intermediate 29 (54.5 g, 0.21 mol) in dry acetone (1000 mL) was added anhydrous K₂CO₃ (43 g, 1.5 eq) and methyl iodide (130 mL, 10 eq). The reaction was stirred at 60° C. for 5 hours. After cooling, the reaction was filtered off and evaporated under reduced pressure. The oil was diluted with dichloromethane and washed with water. The organic layer was dried over Na₂SO₄ and evaporated to dryness to give the title compound (55.7 g, 0.203 mol) as a yellow oil in a 96% yield.

GC/MS: M+ C₁₇H₂₅NO₂ 275

Intermediate 31

4-(4-Isopropyl-2-methoxy-phenyl)-piperidine

To a solution of intermediate 30 (55.7 g, 0.200 mol) in ethanol (500 mL) was added a solution of NaOH (270 mL) in H₂O (270 mL). The reaction was stirred under reflux for 16 hours. After cooling, the reaction was concentrated under reduced pressure, was diluted with dichloromethane and washed with water. The organic layer was dried over sodium sulfate and evaporated to dryness to give the title compound (48.8 g, 0.20 mol) as a yellow oil in a quantitative yield.

GC/MS: M+ C₁₅H₂₃NO 233

Intermediate 32

2-{4-[4-(4-Isopropyl-2-methoxy-phenyl)-piperidin-1-yl]-butyl}-isoindole-1,3-dione

The same method was employed as in the preparation of Intermediate 6 but starting from intermediate 31 gave the title compound as a yellow oil in a quantitative yield.

¹H NMR (CDCl₃, 250 MHz) δ 7.8 (m, 2H), 7.65 (m, 2H), 7-0.05 (d, 1H), 6.7 (dd, 1H), 6.6 (s, 1H), 3.7 (s, 3H), 3.65 (m, 3H), 2.9 (m, 1H), 3.0 (bd, 2H), 2.8 (m, 2H), 2.3 (m, 2H), 2.0 (m, 2H), 1.70-1.5 (m, 6H), 1.2 (d, 6H).

Intermediate 33

4-[4-(4-Isopropyl-2-methoxy-phenyl)-piperidin-1-yl]-butylamine

The same method was employed as in the preparation of intermediate 7 but starting from intermediate 32 gave the title compound as an oil in a 93% yield.

¹H NMR (CDCl₃, 250 MHz) δ 7.05 (m, 1H), 6.7 (dd, 1H), 6.6 (d, 1H), 3.8 (s, 3H), 3.1 (bd, 2H), 2.8 (m, 2H), 2.7 (t, 2H), 2.3 (m, 2H), 2.0-1.3 (m, 12H), 1.15 (d, 6H).

Intermediate 34

1-[4-(2-Ethoxy-4-isopropyl-phenyl)-piperidin-1-yl]-ethanone

To a solution of intermediate 29 (4.85 g, 0.019 mol) in dry acetone (100 mL) was added anhydrous Cs₂CO₃ (12 g, 2 eq) and ethyl iodide (3 mL, 2 eq). The reaction was stirred under reflux for 12 hours. After cooling, the reaction was filtered off and evaporated under reduced pressure to give the title compound (5.4 g, 0.019 mol) as a yellow oil in a quantitative yield.

GC/MS: M+ C₁₈H₂₇NO₂ 289

Intermediate 35

4-(2-Ethoxy-4-isopropyl-phenyl)-piperidine

To a solution of intermediate 34 (5.4 g, 0.019 mol) in ethanol (100 mL) was added a solution of NaOH (25 mL) in H₂O (25 mL). The reaction was stirred under reflux for 16 hours. After cooling, the reaction was concentrated under reduced pressure, was diluted with dichloromethane and washed with water. The organic layer was dried over sodium sulfate and evaporated to dryness to give the title compound (2.6 g, 0.011 mol) as a yellow oil in a 56% yield.

GC/MS: M+ C₁₅H₂₅NO 247

Intermediate 36

2-{4-[4-(2-Ethoxy-4-isopropyl-phenyl)-piperidin-1-yl]-butyl}-isoindole-1,3-dione

The same method was employed as in the preparation of Intermediate 6 but starting from intermediate 35 gave the title compound as a yellow oil in a quantitative yield.

GC/MS: M+ C₂₈H₃₆N₂O₃ 448

Intermediate 37

4-[4-(2-Ethoxy-4-isopropyl-phenyl)-piperidin-1-yl]-butylamine

The same method was employed as in the preparation of intermediate 7 but starting from intermediate 36 gave the title compound as an oil in a 64% yield.

¹H NMR (CDCl₃, 250 MHz) δ 7.05 (d, 1H), 6.7 (dd, 1H), 6.6 (bs, 1H), 3.95 (q, 2H), 3.1 (bd, 2H), 2.8 (m, 2H), 2.7 (m, 2H), 2.3 (m, 2H), 2.0 (m, 2H), 1.8-1.4 (m, 10H), 1.3 (t, 3H), 1.15 (d, 6H).

Intermediate 38

4′-Trifluoromethyl-biphenyl-4-carboxylic Acid

To a solution of 4-Bromo-benzoic acid (28.5 g, 0.14 mol) in toluene (350 mL) were added Tetrakis(triphenylphosphine)palladium(0) (4.93 g, 0.03 eq.), a 2M solution of Na₂CO₃ (71 mL), Lithium chloride (18.3 g, 3 eq.). Then a solution of 4-Trifluoromethylbenzeneboronic acid (30.0 g, 0.158 mol) in EtOH (200 mL) was added and the resulting mixture was stirred at reflux for 16 hours. After evaporation under reduced pressure the residue was taken up in water and the precipitate was filtered off. The solid was treated with a 1N HCl solution, filtered off and dried and was dissolved in a solution of EtOH (700 mL) and THF (400 mL). Filtration through a bed of silica and evaporation gave the title compound (25.0 g, 0.094 mol) as a white solid.

GC/MS: M+ C₁₋₄H₉F₃O₂ 266

Intermediate 39

2,5-Dimethyl-4-(1,2,3,6-tetrahydro-pyridin-4-yl)-phenol

A solution of 2,5-Dimethyl-phenol (12.2 g, 0.1 mol) and 4-Piperidone hydrate hydrochloride (17.0 g, 0.10 mol) in acetic acid (50 mL) was treated with HCl gaz for 15 min. The mixture was stirred at 95° C. for 15 min. After cooling to rt, the mixture was treated with HCl gaz for 5 min. The resulting solution was allowed to stir at rt for 4 days. The solvent was evaporated under reduced pressure to give the title compound as a colorless oil (18.0 g, 0.076 mol) in a 76% yield. White crystals were obtained from iPrOH

MP: 210° C.

Intermediate 40

Acetic Acid 4-(1−acetyl-1,2,3,6-tetrahydro-pyridin-4-yl)-2,5-dimethyl-phenyl Ester

To a solution of intermediate 39 (18.0 g, 0.076 mol) in pyridine (300 mL) was added acetic anhydride (140 mL). The mixture was stirred at rt for 12 hours. The solvents were evaporated under reduce pressure. The oil was diluted with DCM and washed with water. The organic layer was dried over Na₂SO₄ and evaporated to dryness to give the title compound as a yellow oil which was used without further purification.

GC/MS: M+ C₁₇H₂₁NO₃ 287

Intermediate 41

1-[4-(4-Hydroxy-2,5-dimethyl-phenyl)-3,6-dihydro-2H-pyridin-1-yl]-ethanone

To a solution of intermediate 40 in MeOH (300 mL) was added a solution of K₂CO₃ (30.0 g) in H₂O (200 mL) and the mixture was stirred to rt for 12 hours. The solvent was evaporated and the precipitate was filtered off, washed with water and dried to give the title compound (17.0 g, 0.078 mol) in a 88% yield.

MP: 220° C.

Intermediate 42

1-[4-(4-Hydroxy-2,5-dimethyl-phenyl)-piperidin-1-yl]ethanone

To a solution of intermediate 41 (19.0 g, 0.078 mol) in MeOH (1200 mL) and DCM (400 mL) was added Pd/C, 10% (1.9 g) and the reaction was stirred under an atmospheric pressure of hydrogen for 48 hours. The reaction mixture was filtered through a bed of elite. The filtrate was evaporated under reduced pressure to give the title compound (18.5 g, 0.075 mol) as crystals in a 96% yield.

¹H NMR (DMSO, 250 MHz) δ 8.7 (s, 1H), 6.7 (s, 1H), 6.4 (s, 1H), 4.4 (m, 1H), 3.8 (m, 1H), 3.0 (m, 1H), 2.7 (m, 1H), 2.4 (m, 1H), 2.1 (s, 3H), 1.9 (2s, 6H), 1.61.1 (m, 4H).

Intermediate 43

1-{4-[2,5-Dimethyl-4-(pyridin-2-ylmethoxy)-phenyl]-piperidin-1-yl}-ethanone

To a solution of intermediate 42 (4.0 g, 16.2 mmol) in dry acetone was added anhydrous Cs₂CO₃ (13.0 g, 2.4 eq) and 2-Chloromethyl-pyridine hydrochloride (2.92 g, 1.1 eq). The reaction was heated under reflux for 12 hours. After cooling, the solid was filtered off and washed with acetone. The filtrate was evaporated under reduced pressure and diluted with DCM, washed with water and brine. The organic layer was dried over Na₂SO₄ and evaporated to dryness to give the title compound as an orange oil (5.45 g, 16 mmol).

¹H NMR (CDCl₃, 250 MHz) δ 8.5 (m, 1H), 7.7 (m, 1H), 7.4 (m, 1H), 7.1 (m, 1H), 6.8 (s, 1H), 6.6 (s, 1H), 5.1 (s, 2H), 4.7 (m, 1H), 3.9 (m, 1H), 3.1 (m, 1H), 2.8 (m, 1H), 2.6 (m, 1H), 2.3 (bs, 6H), 2.1(s, 3H), 1.7-1.4 (m, 4H).

Intermediate 44

2-(2,5-Dimethyl-4-piperidin-4-yl-phenoxymethyl)-pyridine

To a solution of intermediate 43 (3.8 g, 11 mmol) in EtOH/H₂O (75/15 mL) was added a concentrated NaOH solution (15 mL) and the mixture was stirred to reflux for 16 hours. The EtOH was evaporated and the residue was diluted with DCM, washed with water and dried over MgSO₄ to give the title compound (2.7 g, 9 mmol) as an orange oil.

¹H NMR (CDCl₃, 250 MHz) δ 8.6 (m, 1H), 7.7 (m, 1H), 7.55 (m, 1H), 7.2 (m, 1H), 7 (s, 1H), 6.65 (s, 1H), 5.2 (s, 2H), 3.2 (m, 2H), 2.8 (m, 3H), 2.25 (m, 6H), 1.8-1.5 (m, 5H).

Intermediate 45

2-(44-[2,5-Dimethyl-4-(pyrdin-2-ylmethoxy)-phenyl]-piperidin-1-yl}-butyl)-isoindole-1,3-dione

The same method was employed as in the preparation of intermediate 6 but starting from intermediate 44 gave the title compound as an oil which crystallize from EtOH in a 98% yield.

¹H NMR (DMSO, 250 MHz) δ 8.4 (m, 1H), 7.7 (m, 5H), 7.3 (d, 1H), 7.15 (m, 1H), 6.7 (s, 1H), 6.6 (s, 1H), 5.0 (bs, 2H), 3.5 (m, 2H), 2.7 (m, 2H), 2.1 (bt, 2H), 2.0 (2s, 6H), 1.5-1.2 (m, 1H).

Intermediate 46

4-{4-[2,5-Dimethyl-4-(pyridin-2-ylmethoxy)-phenyl]-pipenidin-1-yl}-butylamine

The same method was employed as in the preparation of intermediate 7 but starting from intermediate 45 gave the title compound as a gummy solid in a 95% yield.

¹H NMR (CDCl₃, 250 MHz) δ 8.5 (m, 1H), 7.6 (m, 1H), 7.4 (m, 1H), 7.0 (m, 1H), 6.9 (s, 1H), 6.5 (s, 1H), 5.0 (s, 2H), 2.9 (m, 2H), 2.6 (t, 2H), 2.45 (m, 1H), 2.25 m, 2H), 2.2 (2s, 6H), 1.9 (m, 2H), 1.7-1.3 (m, 10H).

Intermediate 47

4-Benzo[1,3]dioxol-5-yl-1-benzyl-piperidin-4-ol

Ref: WO97/09311

A solution of 4-Bromo-1,2-(methylenedioxy)-benzene (36.6 g, 0.182 mol) in THF (250 mL) was cooled to −78° C. and treated with nBuLi (2.0 M in cyclohexane, 100 mL, 1.2 eq.). The resulting mixture was stirred for 2 hours at −55° C. At −78° C. a solution of 1-Benzyl-4-piperidone (34.4 g, 1 eq.) in THF (100 mL) was added. The resulting mixture was allowed to stir at −40° C. for 2 hours and allowed to warm up to rt. Addition of a saturated ammonium chloride solution, extraction with EtOAc, drying over Na₂SO₄ and evaporation under reduced pressure gave the title compound as an oil which was crystallized from Et₂O (38.0 g, 0.122 mol).

MP: 140° C.

Intermediate 48

4-Benzo[1,3]dioxol-5-yl-1-benzyl-1,2,3,6-tetrahydro-pyridine

A solution of intermediate 47 (32.0 g, 0.109 mol) in toluene (1000 mL) was treated with pTsOH (22.5 g, 1.2 eq.) and was stirred to reflux for 4 hours. Addition of a saturated NaHCO₃ solution, extraction with EtOAc, drying over Na₂SO₄ and evaporation under reduced pressure gave the title compound as an oil (31.0 g, 0.105 mol).

GC/MS: M+ C₁₉H₁₉NO₂ 293

Intermediate 49

4-Benzo[1,3]dioxol-5-yl-piperidine

A solution of intermediate 48 (31.0 g, 0.105 mol) in MeOH (350 mL) was treated with Pd/C, 10% (2.5 g) under an atmospheric pressure of hydrogen. The resulting solution was allowed to stir at 50° C. for 24 hours. The reaction mixture was filtered through a bed of celite. The filtrate was evaporated under reduced pressure to give the title compound as an oil (15.0 g, 0.075 mol).

¹H NMR (CDCl₃, 250 MHz) δ 6.6 (m, 3H), 5.8 (s, 2H), 4.9 (m, 1H), 3.2 (m, 2H), 2.7 (m, 2H), 1.5-1.8 (m, 4H).

Ref: Bioorg. Med. Chem. Lett. (1992), 2(2), 165-70

Intermediate 50

2-[4-(4-Benzo[1,3]dioxol-5-yl-piperidin-1yl)-butyl]-isoindole-1,3-dione

A solution of intermediate 49 (2.0 g, 10 mmol) in DMF (100 mL) was treated with K₂CO₃ (1.7 g, 1.2 eq.) and N-(4-Bromobutyl)-phtalimide (3.11 g, 1.1 eq.). The resulting mixture was stirred at 100° C. for 16 hours. After cooling to rt the reaction mixture was filtered off. The filtrate was evaporated off. The title compound (2.0 g, 4.65 mmol) was obtained as an orange powder.

¹H NMR (CDCl₃, 250 MHz) δ 7.8 (m, 2H), 7.6 (m, 2H), 6.6 (m, 3H), 5.9 (s, 2H), 3.7 (m, 2H), 2.9 (m, 2H), 2.3 (m, 3H), 2 (m, 2H), 1.7-1.4 (m, 8H).

Intermediate 51

4-(4-Benzo[1,3]dioxol-5-yl-piperidin-1yl)-butylamine

The same method was employed as in the preparation of intermediate 7 but starting from intermediate 50 gave the title compound as a yellow oil in a 91% yield.

¹H NMR (CDCl₃, 250 MHz) δ 6.6 (m, 3H), 5.8 (s, 2H), 2.9 (m, 2H), 2.6 (m, 2H), 2.3 (m, 3H), 1.9 (m, 2H), 1.7-1.3 (m, 8H).

Intermediate 52

1-Benzyl-4-naphthalen-2-yl-piperidin-4-ol

Ref: WO 9748698 A1

The same method was employed as in the preparation of intermediate 47 but starting from 2-Bromonaphthalene gave the title compound as an oil in a 77% yield.

¹H NMR (CDCl₃, 250 MHz) δ 7.9 (s, 1H), 7.8 (m, 3H), 7.7 (m, 1H), 7.5-7.15 (m, 7H), 3.6 (s, 2H), 2.85 (m, 2H), 2.60 (m, 2H), 2.25 (m, 2H), 1.9-1.6 (m, 3H).

Intermediate 53

1-Benzyl-4-naphthalen-2-yl-1,2,3,6-tetrahydro-pyridine

Ref: WO 9709311 A1

The same method was employed as in the preparation of intermediate 48 but starting from intermediate 52 gave the title compound as an oil in a 94% yield.

¹H NMR (CDCl₃, 250 MHz) δ 7.75 (m, 3H), 7.5 (d, 1H), 7.3 (m, 8H), 6.25 (m, 1H), 3.6 (s, 2H), 3.2 (m, 2H), 2.8-2.6 (m, 4H).

Intermediate 54

4-Naphthalen-2-yl-piperidine

Ref: WO 9737979 A1

The same method was employed as in the preparation of intermediate 49 but starting from intermediate 53 gave the title compound as an oil in a 87% yield.

¹H NMR (CDCl₃, 250 MHz) δ 7.7 (m, 3H), 7.6 (m, 1H), 7.3 (m, 3H), 3.2 (m, 2H), 2.7 (m, 3H), 1.9-1.4 (m, 4H).

Intermediate 55

2-[4-(4-Naphthalen-2-yl-piperidin-1yl)-butyl]-isoindole-1,3-dione

The same method was employed as in the preparation of intermediate 6 but starting from intermediate 54 gave the title compound as an oil in a 40% yield.

¹H NMR (CDCl₃, 250 MHz) δ 7.8-7.5 (m, 8H), 7.3 (m, 3H), 3.7 (t, 2H), 3.05 (m, 2H), 2.6 (m, 1H), 2.4 (m, 2H), 2.1-1.4 (m, 10H).

Intermediate 56

4-(4-Naphtalen-2-yl-piperidin-1yl)-butylamine

The same method was employed as in the preparation of intermediate 7 but starting from intermediate 55 gave the title compound as an oil in a 84% yield.

¹H NMR (CDCl₃, 250 MHz) δ 7.7 (m, 3H), 7.6 (s, 1H), 7.35 (m, 3H), 3.0 (bd, 2H), 2.6 (m, 3H), 2.4 (m, 2H), 2.8-2.2 (m, 6H), 1.5 (m, 6H).

Intermediate 57

4-(5,6,7,8-Tetrahydro-naphthalen-2-yl-piperidine

A solution of intermediate 53 (3.7 g, 12 mmol) in EtOH (200 mL) and a concentrated HCl solution (20 mL) was treated with Pd/C, 10% (0.5 g) under a pressure of hydrogen (10 bars). The resulting solution was allowed to stir at 50° C. for 24 hours. After cooling, the reaction mixture was filtered through a bed of celite. The filtrate was evaporated under reduced pressure, diluted with DCM, washed with a 1N NaOH solution and brine. The organic layer was dried over Na₂SO₄ and evaporated to dryness to give the title compound as an oil (2.1 g, 9.8 mmol) in a 85% yield.

GC/MS: M+ C₁₅H₂₁N 215

Intermediate 58

2-{4-[4-(5,6,7,8-Tetrahydro-naphthalen-2-yl)-piperidin-1yl]-butyl}-isoindole-1,3-dione

The same method was employed as in the preparation of intermediate 6 but starting from intermediate 57 gave the title compound as an oil in a 35% yield.

LC/MS (APCI): [M+H⁺] C₂₇H₃₂N₂O₂ 417

Intermediate 59

4-[4-(5,6,7,8-Tetrahydro-naphtalen-2-yl)-piperidin-1yl]-butylamine

The same method was employed as in the preparation of intermediate 47 but starting from intermediate 58 gave the title compound as an oil in a 84% yield.

¹H NMR (CDCl₃, 250 MHz) δ 6.8 (m, 3H), 3.0 (bd, 2H), 2.7 (m, 6H), 2.3 (m, 3H), 1.9 (m, 2H), 1.7 (m, 10H), 1.5 (m, 2H).

Intermediate 60

1-Benzyl-4-naphthalen-1-yl-piperidin-4-ol

The same method was employed as in the preparation of intermediate 47 but starting from 1-Bromo-naphthalene gave the title compound as an oil in a 83% yield.

¹H NMR (CDCl₃, 250 MHz) δ 8.9 (m, 1H), 7.75 (m, 1H), 7.65 (d, 1H), 7.5-7.0 (m, 9H), 3.65 (s, 2H), 2.8 (m, 2H), 2.15 (m, 2H), 2.4-2.1 (m, 4H), 1.7 (bs, 1H).

Refs: EP 372776 and WO97/48698

Intermediate 61

1-Benzyl-4-naphthalen-1-yl-1,2,3,6-tetrahydro-pyridine

A solution of intermediate 60 (14.0 g, 44.0 mmol) in DCM (150 mL) was treated with TFA (70 mL, 20 eq.) and triethyl silane (280 mL, 40 eq.) at rt. The resulting solution was allowed to stir at rt for 24 hours. The solvent was evaporated under reduced pressure. The residue was filtered through a bed of silica to give the title compound (8.3 g, 27.7 mmol) as an oil.

¹H NMR (CDCl₃, 250 MHz) δ 8 (m, 1H), 7.8 (m, 1H), 7.7 (bd, 1H), 7.3 (m, 9H), 5.75 (m, 1H), 3.7 (s, 2H), 3.2 (m, 2H), 2.75 (t, 2H); 2.5 (m, 2H).

Ref: WO97/09311 A1

Intermediate 62

4-Naphthalen-1-yl-piperidine

Refs: EP 466585 A1 and EP 372776 A2

The same method was employed as in the preparation of intermediate 49 but starting from intermediate 61 gave the title compound as an oil in a quantitative yield.

¹H NMR (CDCl₃, 250 MHz) δ 8.05 (m, 1H), 7.8 (m, 1H), 7.6 (bd, 1H), 7.35 (m, 4H), 3.4 (m, 1H), 3.25 (bd, 2H), 2.7 (m, 2H), 1.95 (m, 2H), 1.7 (m, 4H).

Intermediate 63

2-[4-(4-Naphthalen-1-yl-piperidin-1-yl)-butyl]-isoindole-1,3-dione

The same method was employed as in the preparation of intermediate 6 but starting from intermediate 62 gave the title compound as an oil in a 41% yield.

¹H NMR (CDCl₃, 250 MHz) δ 8 (d, 1H), 7.75 (m, 2H), 7.6 (m, 2H), 7.4 (m, 6H), 3.65 (t, 2H), 3.3 (m, 1H), 3.1 (bd, 2H), 2.5 (m, 2H), 2.2 (m, 2H), 1.90 (m, 4H), 1.75-1.5 (m, 4H).

Intermediate 64

4-(4-Naphtalen-1-yl-piperidin-1yl)-butylamine

The same method, was employed as in the preparation of intermediate 7 but starting from intermediate 63 gave the title compound as an oil in a 71% yield.

¹H NMR (CDCl₃, 250 MHz) δ 8.05 (m, 1H), 7.8 (m, 1H), 7.6 (m, 1H), 7.35 (m, 4H), 3.25 (m, 1H), 3.05 (bd, 2H), 2.7 (t, 2H), 2.4 (m, 2H), 2.1 (m, 2H), 1.85 (m, 4H), 1.6-1.2 (m, 6H).

Intermediate 65

1-[4-(2-trifluoroethoxy-4-methyl-phenyl)-piperidin-1-yl]-ethanone

To a solution of the intermediate 9 (4.6 g, 20 mmol) in DMF (150 mL) was added Cs₂CO₃ (8.13 g, 25 mmol) and trifluoroethyltriflate (5.0 g, 21.5 mmol). The mixture was then stirred at 50° C. during 24 hours. After cooling, the mixture was filtrated off and the cake was generously washed with DCM. The filtrate was evaporated under vacuo to yield the title compound (5 g, 15.8 mmol) as a oil.

GC/MS: M+ C₁₆H₂₀NO₂F₃ 315

Intermediate 66

4-(2-trifluoroethoxy-4-methyl-phenyl)-piperidine

The same method was employed as in the preparation of intermediate 11 but starting from intermediate 65 gave the title compound as an orange oil in a 95% yield.

LC/MS: [M+H⁺] 274 C₁₄H₁₈F₃NO

Intermediate 67

4-Hydroxy-4-(2-methylsulfanyl-phenyl)-piperidine-1-carboxylic Acid ter-butyl Ester

The same method was employed as in the preparation of intermediate 47 but starting from 1-Bromo-2-methylsulfanyl-benzene and 1-Boc-4-piperidone gave the title compound as a colorless oil in a quantitative yield.

GC/MS: M+ C₁₇H₂₅NSO₃ 323

Intermediate 68

4-(2-Methylsulfanyl-phenyl)-piperidine

The same method was employed as in the preparation of intermediate 4 but starting from intermediate 67. A mixture of the title compound and the corresponding 1,2,3,6-tetrahydro-pyridine was obtained as an oil in a 89% yield. The crude compound was used in the next step-without purification.

Intermediate 69

2-{4-[4-(2-Methylsulfanyl-phenyl)-piperidin-1-yl]-butyl}-isoindole-1,3dione

The same method was employed as in the preparation of intermediate 6 but starting from intermediate 68. After separation by flash chromatography, a pure fraction was isolated to give the title compound.

GC/MS: M+ C₂₄H₂₈N₂SO₂ 408

Intermediate 70

4-[4-(2-Methylsulfanyl-phenyl)-piperidin-1-yl]-butylamine

The same method was employed as in the preparation of intermediate 7 but starting from intermediate 69 gave the title compound as a yellow oil in a 85% yield.

GC/MS: M+ C₁₆H₂₆N₂S 278

Intermediate 71

1-[4-(1-Methyl-1H-indol-3-yl)-piperidin-1-yl]-ethanone

To a solution of the available 1-[4-(1H-Indol-3-yl)-piperidin-1-yl]-ethanone, (1.0 g, 4.2 mmol) in dry THF (50 mL) was added NaH 60% (0.170 g, 1.1 eq.) and methyl iodide (0.64 g, 1.1 eq.). The mixture was stirred at rt for 18 hours. After cooling, the mixture was washed with water and extracted off with EtOAc and dried over Na₂SO₄ to give after evaporation, the title compound (1.0 g, 3.9 mmol) as yellow crystals in a quantitative yield, after cristallization in Et₂O.

GC/MS: M+ C₁₆H₂₀N₂O 256

Intermediate 72

1-Methyl-3-piperidin-4-yl-1H-indole

To a solution of intermediate 71 (1.0 g, 3.9 mmol) in EtOH (20 mL) was added a NaOH/H₂O (1/1) solution (7 mL) and the reaction was stirred to reflux for 16 hours. After cooling, the reaction was concentrated in vacuo, and the residue was diluted with water and treated with a 1N HCl solution until PH=3, extracted with DCM. The organic phase was then dried over Na₂SO₄ and evaporated off. The title compound was obtained as a yellow oil (0.52 g, 2.4 mmol) in a 63% yield.

¹H NMR (CDCl₃, 300 MHz) δ 7.5 (d, 1H), 7.1 (m, 2H), 6.9 (m, 1H), 6.5 (s, 1H), 3.6 (s, 3H), 3.0 (m, 2H), 2.8 (m, 1H), 2.5 (m, 2H), 1.8 (m, 2H), 1.5 (m, 2H).

Intermediate 73

2-{4-[4-(1-Methyl-1H-indol-3-yl)-piperidin-1-yl]-butyl}-isoindole-1,3-dione

To a solution of intermediate 72 (0.52 g, 2.4 mmol) in solution in acetone (20 mL) was added potassium carbonate (0.66 g, 2.0 eq.) and N-4-bromobutyl phtalimide (0.76 g, 1.1 eq.). The reaction was stirred to reflux for 16 hours. After cooling, the reaction was filtered off and the solvent was removed in vacuo. After purification by flash chromatography, using DCM/MeOH (90/10) as eluent, the title compound was obtained as a yellow oil (0.8 g, 1.9 mmol) in a 80% yield.

¹H NMR (CDCl₃, 300 MHz) δ 7.9 (m, 2H), 7.8 (m, 2H), 7.5 (d, 1H), 7.1 (m, 2H), 6.9 (m, 1H), 6.5 (s, 1H), 3.6 (m, 5H), 3.0 (m, 2H), 2.8 (m, 1H), 2.5 (m, 2H), 2.2 (m, 2H), 1.8 (m, 4H), 1.5 (m, 4H).

Intermediate 74

4-[4-(1-Methyl-1H-indol-3-yl)-pipenidin-1-yl]-butylamine

To a solution of intermediate 73 (0.8 g, 1.9 mmol) in solution in MeOH (20 mL) was added hydrazine hydrate (0.5 mL, 5.0 eq.) and the reaction was stirred to reflux for 16 hours.

After evaporation under reduced pressure, the residue was taken up in water and treated with a concentrated NaOH solution until PH>12. Extraction with DCM, drying over Na₂SO₄ and filtration gave the title compound (0.4 g, 1.4 mmol) as a yellow oil in a 74% yield.

¹H NMR (CDCl₃, 300 MHz) 57.5 (d, 1H), 7.1 (m, 2H), 6.9 (m, 1H), 6.5 (s, 1H), 3.6 (m, 5H), 3.0 (m, 2H), 2.8 (m, 1H), 2.5 (m, 2H), 2.2 (m, 2H), 1.8 (m, 4H), 1.5 (m, 4H).

Intermediate 75

2-(4,4-Diethoxy-butyl)-isoindole-1,3-dione

To a solution of Isobenzofuran-1,3-dione (10.0 g, 0.068 mol) in toluene (200 mL) were added 4-Aminobutyraldehyde diethyl acetal (14.5 g, 1.2 eq.) and TEA (14.0 mL, 1.5 eq.). The reaction was stirred to reflux for 16 hours. The toluene was removed under vacuo and the residue was dissolved in Et₂O and washed with water. The organic phase was dried over Na₂SO₄ and concentrated under vacuo to give the title compound (21.0 g, 1.0 eq.) as a oil in a quantitative yield.

GC/MS: M+ C₁₆H₂₁NO₄ 291

Intermediate 76

4-(1,3-Dioxo-1,3-dihydro-isoindole-2-yl)-butyraldehyde

Ref: J. Med. Chem. (1992), 35, 3239-46.

To a solution of intermediate 75 (21.0 g, 0.068 mol) in acetone (200 mL) was added a 1N HCl solution (100 mL) and the reaction was stirred to reflux for 2 hours. The solvent was then evaporated and a 1N NaOH solution (200 mL) was added. The product was extracted with DCM and the organic phase was dried over Na₂SO₄ and concentrated under vacuo. The title compound was obtained as a yellow oil (8.4 g, 0.039 mol) in a 59% yield.

¹H NMR (CDCl₃, 300 MHz) δ 9.6 (s, 1H), 7.8 (m, 2H), 7.4 (m, 2H), 3.6 (t, 2H), 2.4 (t, 2H), 1.8 (m, 2H).

Intermediate 77

2-{4-[4-(1-Indol-3-yl)-piperidin-1-yl]-butyl}-isoindole-1,3-dione

To a solution of the available 3-Piperidine-4-yl-1-H-indole (1.0 g, 5.0 mmol) in dry THF (50 mL) was added the intermediate 76 (1.08 g, 1.0 eq.). The reaction was stirred at rt for 30 min and AcOH (1.5 eq) was added. Then a 1M NaBH₃CN solution in THF (1.2 eq.) was added and the reaction was stirred for 24 hours at rt. Then, water was added (20 mL). The organic phase was dried over Na₂SO₄ and concentrated under vacuo. After purification by flash chromatography, the title compound was obtained (0.5 g, 1.2 mmol) as a yellow oil in a 25% yield.

GC/MS: M+ C₂₅H₂₇N₃O₃ 401

Intermediate 78

4-[4-(1−Indol-3-yl)-piperidin-1-yl]-butylamine

The same method was employed as in the preparation of intermediate 7 but starting from intermediate 77 gave the title compound as an oil (0.370 g, 1.4 mmol) in a quantitative yield.

¹H NMR (CDCl₃, 300 MHz) δ 8.0 (s, 1H), 7.6 (d, 1H), 7.3 (d, 1H), 7.6 (m, 2H), 6.9 (s, 1H), 3.1 (m, 2H), 2.8 (m, 1H), 5 (t, 2H), 2.2 (m, 2H), 2.0 (m, 4H), 1.8-1.2 (m, 6H).

Intermediate 79.

3-Bromo-benzo[b]thiophene

ref: JACS, 72, 574, (1950)

To a solution of benzothiophene (89.0 g, 0.66 mol) in chloroform (500 mL) was added sodium acetate (93.1 g, 1.7 eq.) and at 0° C., bromine (35.6 mL, 1.0 eq.) in solution in chloroform (100 mL) was added dropwise. The reaction was then stirred at rt during 3 hours. H₂O (300 mL) was added and the organic phase was then washed with a 1N NaOH solution, dried over Na₂SO₄ and concentrated under vacuo. The title compound was obtained as a yellow oil in a 86% yield after purification by distillation.

Peb: 102° C., P=2 mbars

Intermediate 80

4-Benzo[b]thiophen-3-yl-1-benzyl-piperidin-4-ol

At −78° C., to a solution of intermediate 79 (40.47 g, 0.19 mol) in dry THF (500 mL) was added dropwise nBuLi (100 mL, 1.0 eq., 2M in solution in cyclohexane) during 15 min. The reaction was then stirred for 4 hours at −78° C. and the N-Benzyl-piperidone (27.9 g, 10 eq) was added in solution in dry THF (250 mL). The reaction was then stirred at rt during 1 night. A saturated NH₄Cl solution (400 mL) was added, the organic phase was then decanted and the aqueous phase was extracted with AcOEt. The combined organic phase was dried over Na₂SO₄ and concentrated under vacuo. The title compound was obtained after purification by flash chromatography using DCM/MeOH 98/2 as eluent.

The title compound was obtained as a yellow solid (49.4 g, 0.153 mol) in a 80% yield.

¹H NMR (CDCl₃, 300 MHz) δ 7.84 (d, 1H), 7.72 (d, 1H), 7.38 (m, 7H), 7.22 (s, 1H), 3.6 (s, 2H), 2.78 (m, 2H), 2.55 (m, 2H), 2.27 (m, 2H), 2.03 (m, 2H).

Intermediate 81

4-Benzo[b]thiophen-3-yl-1-benzyl-1,2,3,6-tetrahydro-pyridine

To a solution of intermediate 80 (49.4 g, 0.153 mol) in AcOH (200 mL) was added a concentrated HCl solution (60 mL). The reaction was then stirred to reflux for 6 hours and at rt for 48 h. The formed precipitate was filtered and the filtrate was evaporated off. The residue was taken off in DCM and filtered off. The combined solids were washed with DCM, dissolved in concentrated NaOH solution and extracted with DCM. The organic phase was dried over Na₂SO₄ and concentrated under vacuo. The title compound was obtained as a yellow solid (22.7 g, 0.074 mol) in a 49% yield.

¹H NMR (CDCl₃, 300 MHz) δ 7.77 (d, 1H), 7.68 (d, 1H), 7.43-7.28 (m, 7H), 7.15 (s, 1H), 6.22 (m, 1H), 3.69 (s, 2H), 3.23 (m, 2H), 2.78 (m, 2H), 2.67 (m, 2H).

Intermediate 82

4-Benzolb]thiophen-3-yl-1-benzyl-piperidine

To a solution of intermediate 81 (22.7 g, 0.074 mol) in AcOH (250 mL) was added Pd/C 10% (8.5 g) and the reaction was stirred under a pressure of hydrogen (10 bars) at 60° C. for 24 hours.

The reaction mixture was filtered through a bed of celite. The filtrate was evaporated under reduced pressure to give the title compound as a colorless solid (18.9 g, 0.061 mol) in a 83% yield.

MP: 90° C.

Intermediate 83

4-Benzo[b]thiophen-3-yl-piperidine

Ref: Eur. Pat. Appl. (1996), EP 699675

To a solution of vinyl chloroformate (5.88 mL, 1.5 eq.) in DCM (100 mL) at 0° C., was added a solution of intermediate 82 (14.16 g, 0.046 mol) in DCM (200 mL). The reaction was stirred to reflux for 4 hours then cooled to o° C. and treated with HCl gaz for 15 min and evaporated off. The residue was dissolved in MeOH (250 mL) and stirred at 60° C. for 5 hours. After evaporation of MeOH, the residue was purified by flash chromatography using MeOH/DCM, 98/2 and 90/10 to give the title compound as a white solid (5.44 g, 0.025 mol) in a 54% yield.

GC/MS: M+ C₁₃H₁₅NS 217

Intermediate 84

2-[4-(4-Benzo[b]thiophen-3-yl-piperidin-1-yl)-butyl]-isoindole-1,3-dione

The same method was employed as in the preparation of intermediate 6 but starting from intermediate 83 gave the title compound as a white solid (4.5 g, 0.011 mol.) in a 43% yield.

¹H NMR (CDCl₃, 300 MHz) δ 8.0 (m, 2H), 7.9 (m, 4H), 7.4 (m, 2H), 7.2 (s, 1H), 3.9 (t, 2H), 3.2 (m, 2H), 3.05 (m, 2H), 2.6 (m, 2H), 1.8-1.2 (m, 9H).

Intermediate 85

4-(4-Benzo[b]thiophen-3-yl-piperidin-1-yl)-butylamine

The same method was employed as in the preparation of intermediate 7 but starting from intermediate 84 gave the title compound as a yellow solid (0.36 g, 1.25 mmol) in a 12% yield.

¹H NMR (DMSO d₆, 300 MHz) δ 7.9 (d, 1H), 7.8 (d, 1H), 7.4 (m, 2H), 7.3 (s, 1H), 3.6 (m, 2H), 3.1 (m, 7H), 2.8 (m, 2H), 2.2 (m, 4H), 1.8-1.6 (m, 4H).

Intermediate 86

2-{4-[4-(2,4-Dimethoxy-phenyl)-piperazin-1-yl]-butyl}-isoindole-1,3-dione

The same method was employed as in the preparation of intermediate 6 but starting from 1-(2,4-Dimethoxy-phenyl)-piperazine. The solution was filtered off and was evaporated off. The residue was flash chromatographed using DCM/MeOH (90/10) to give the title compound as an oil in a quantitative yield.

¹H NMR (CDCl₃, 250 MHz) δ 7.85 (m, 2H), 7.7 (m, 2H), 6.85 (d, 1H), 6.4 (m, 2H), 3.8 (d, 6H), 3.7 (m, 4H), 3.0 (m, 4H), 2.6 (m, 4H), 2.45 (t, 2H), 1.75 (m, 2H), 1.6 (m, 2H).

Intermediate 87

4-[4-(2,4-Dimethoxy-phenyl)-piperazin-1-yl]-butylamine

The same method was employed as in the preparation of intermediate 7 but starting from the intermediate 86 gave the title compound as a yellow oil in a quantitative yield.

¹H NMR (CDCl₃, 250 MHz) δ 6.9 (d, 1H), 6.4 (m, 2H), 3.8 (d, 6H), 3.1 (m, 4H), 2.75 (t, 2H), 2.55 (m, 4H), 2.45 (t, 2H), 1.6 (m, 6H).

Intermediate 88

1-(2,4-Dimethoxy-phenyl)-[1,4]diazocane

A solution of [1,4]Diazocane (4.6 g, 0.046 mol) in THF (60 mL) was cooled to 0° C. and a solution of nBuLi (2.0 M in hexane, 25.3 mL, 1.1 eq.) was added dropwise at 0° C. and stirred at rt for 2 hours. A solution of 1-Bromo-2,4-dimethoxy-benzene (10.0 g, 0.046 mol) in THF (50 mL) was added at rt and the resulting mixture was stirred to reflux for 4 hours. After cooling to rt, a 1N HCl solution (100 mL) was added to the resulting mixture. Extraction with toluene, drying over Na₂SO₄ and evaporation under reduced pressure gave a residue that was flash chromatographed using DCM/MeOH/iPr₂NH (80/20/2) as eluent. The title compound (3.0 g, 12.7 mmol) was isolated as a brown oil in a 28% yield.

¹H NMR (CDCl₃, 250 MHz) δ 5.8 (m, 3H), 3.8 (s, 6H), 3.5 (m, 4H), 3.0 (m, 2H), 2.75 (m, 2H), 1.8 (m, 3H).

Intermediate 89

2-{4-[4-(2,4-Dimethoxy-phenyl)-[1,4]diazocan-1-yl]-butyl}-isoindole-1,3-dione

The same method was employed as in the preparation of intermediate 6 but starting from intermediate 88 to give the title compound as a yellow oil in a 75% yield.

The crude compound was used in the next step without purification.

Intermediate 90

4-[4-(2,4-Dimethoxy-phenyl)-[1,4]diazocan-1-yl]-butylamine

The same method was employed as in the preparation of intermediate 7 but starting from intermediate 89 to give the title compound as a yellow oil in a 84% yield.

¹H NMR (CDCl₃, 250 MHz) δ 5.8 (m, 3H), 3.4 (s, 6H), 3.4 (m, 4H), 2.3-2.7 (m, 8H), 1.8 (m, 2H), 1.35 (m, 6H).

Intermediate 91

2-Ethoxy-4-methyl-1-nitro-benzene

To a solution of 5-Methyl-2-nitrophenol (50.0 g, 0.33 mol) in dry acetone (400 mL) was added K₂CO₃ (55.0 g, 1.2 eq.) and ethyl iodide (51 mL, 2 eq.) and the mixture was stirred under reflux for 16 hours. The solution wag filtered off and evaporated off. The title compound (50.0 g, 0.276 mol) was obtained as yellow crystals in a 85% yield.

GC/MS: M+ C₉H₁₁NO₃ 181

Intermediate 92

2-Ethoxy-4-methyl-phenylamine

To a solution of intermediate 91 (50.0 g, 0.276 mol) in EtOH (1000 mL) was added Pd/C 10% (2.5 g) and the mixture was stirred under Patm 12 at rt for 16 hours. The solution was then filtered off on a bed of celite and the solvent was evaporated off. The oil was treated with a 1N HCl solution (250 mL) and th starting material which had not react was extracted with Et₂O. The aqueous phase was neutralized with a 1N NaOH solution and the compound was extracted with DCM. The organic layer was dried over Na₂SO₄ and evaporated to dryness to give the title compound (11.0 g, 0.073 mol) as an oil in 26% yield.

GC/IMS: M+ C₉H₁₃NO 151

Intermediate 93

1-[4-(2-Ethoxy-4-methyl-phenylamino)-piperidin-1-yl]-ethanone

To a solution of intermediate 92 (11.0 g, 73.0 mmol) in MeOH (100 mL) was added the N-Acetyl piperidone (10.3 g, 1.0 eq.). The reaction was stirred at rt for 30 min and AcOH (1.5 eq) was added. Then sodium triacetoxyborohydride (1.2 eq.) was added and the reaction was stirred for 24 hours at reflux. After cooling, the solvent was evaporated. The brown oil was treated in acidic and basic conditions to give the title compound as a colorless oil (6.8 g, 0.024 mol) in 33% yield.

GC/MS: M+ C₁₆H₂₄N₂O₂ 276

Intermediate 94

(2-Ethoxy-4-methyl-phenyl)-piperidin-4-yl-amine

To a solution of the intermediate 93 (1.0 g, 3.6 mmol) in EtOH (30 mL) was added a 1/1 concentrated NaOH solution and H₂O (10 mL). The resulting mixture was stirred to reflux for 16 hours. After cooling and evaporation under reduced pressure, the residue was taken up in DCM and washed with water and brine. The organic phase was dried over Na₂SO₄ and evaporated off to give the title compound (0.81 g, 3.4 mmol) as an oil in a 96% yield.

¹H NMR (CDCl₃, 300 MHz) δ 6.4 (m, 3H), 4.0 (m, 3H), 3.2 (m, 1H), 3.05 (m, 2H), 2.7 (m, 2H), 2.2 (s, 3H), 2.1-1.9 (m, 5H), 1.3 (t, 3H).

Intermediate 95

2-{4-[4-(2-Ethoxy-4-methyl-phenylamino)-piperidin-1-yl]-butyl}-isoindole-1,3-dione

To solution of intermediate 94 (0.81 g, 3.4 mmol) in acetone (10 mL) was treated with K₂CO₃ (0.1 g, 2.0 eq.) and N-(4-Bromobutyl)-phtalimide (2.0 g, 2.0 eq.). The resulting mixture was stirred under reflux for 16 hours. After cooling to rt, the reaction mixture was filtered off. The cake was washed with acetone. The filtrate was evaporated off. The residue was diluted in DCM and washed with water. The organic phase was dried over Na₂SO₄ and evaporated off. The oil was purified by flash chromatography using DCM/MeOH, 98/2 and DCM/MeOH, 9/1 as eluent to give th title compound (1.37 g, 3 mmol) as an oil in a 91% yield.

¹H NMR (CDCl₃, 300 MHz) δ 8.1 (m, 2H), 7.9 (m, 2H), 6.8 (m, 3H), 4.2 (q, 2H), 3.9 (t, 2H), 3.4 (m, 1H), 3.05 (m, 2H), 2.6 (m, 2H), 2.4 (s, 3H), 2.3 (m, 4H), 1.9-1.7 (m, 6H), 1.4 (t, 3H).

Intermediate 96

2-ethoxy-4-methyl-phenyl [1-(4-Amino-butyl)-piperidin-4-yl]-(2-ethoxy-4-methylphenyl)-amine

A solution of intermediate 95 (1.37 g, 3.15 mmol) in EtOH (50 mL) was treated with hydrazine hydrate (800 μL, 5.0 eq.). The resulting mixture was stirred at 50° C. for 16 hours. After evaporation under reduced pressure the residue was taken up in water and treated with a concentrated HCl solution until PH=3. The white precipitate was filtered off, washed with water and the filtrate was treated with a concentrated NaOH solution until PH=13. Extraction with DCM, drying over Na₂SO₄ and filtration gave the title compound (0.89 g, 2.9 mmol) as an oil in a 93% yield.

¹H NMR (CDCl₃, 300 MHz) δ 6.5 (m, 3H), 4.0 (q, 2H), 3.2 (m, 1H), 2.8 (m, 2H), 2.6 (m, 2H), 2.3 (m, 2H), 2.2 (s, 3H), 2.0 (m, 4H), 1.4 (m, 6H), 1.4 (t, 3H).

Intermediate 97

1-Benzyl-4-(naphtalen-1-yloxy)-piperidine

The tributylphosphine (2.11 g, 10.5 mmol) was added to a solution of TMAD (1.8 g, 1.0 eq.) in dry THF (20 mL) at rt. When the mixture was colourless, 1-naphtol (1.5 g, 1.0 eq.) and N-Benzylpiperidol (2.0 g, 1.0 eq.) were added and the resulting mixture was heated at 60° C. for 48 hours. After cooling, the mixture was diluted with EtOAc and washed with water, dried over Na₂SO₄, filtrated off and evaporated off. The residue was purified by flash chromatography using DCM/MeOH 95/05 as eluent to give the title compound as a yellow oil in a quantitative yield.

GC/MS: M+ C₂₂H₂₃NO 317

Intermediate 98

4-(Naphtalen-1-yloxy)-piperidine

A solution of intermediate 97 (3.8 g, 12 mmol) in MeOH (200 mL) was treated with Pd/C, 10% (0.38 g) under hydrogen. The resulting solution was allowed to stir at rt for 24 hours. The reaction mixture was filtered through a bed of celite. The filtrate was evaporated under reduced pressure to give the title compound as a yellow oil (0.6 g, 2.64 mmol) in a 22% yield.

GC/MS: M+ C₁₅H₁₇NO 227

Intermediate 99

2-{4-[4-(Naphtalen-1-yloxy)-piperidin-1-yl]-butyl}-isoindole-1,3-dione

The same method was employed as in the preparation of intermediate 6 but starting from intermediate 98 gave the title compound as a yellow oil (0.63 g, 1.5 mmol) in a 56% yield.

GC/IMS: M+ C₂₇H₂₈N₂O₃ 428

Intermediate 100

4-[4-(Naphtalen-1-yloxy)-piperidin-1-yl]-butylamine

The same method was employed as in the preparation of intermediate 7 but starting from intermediate 99 gave the title compound as a yellow oil (0.2 g, 0.7 mmol) in a 47% yield.

¹H NMR (CDCl₃, 300 MHz) δ 8.25 (d, 1H), 7.8 (d, 1H), 7.25 (m, 7H), 6.8 (d, 1H), 4.4 (m, 1H), 2.7 (m, 4H), 2.3 (m, 4H), 1.9 (m, 3H), 1.3 (m, 8H).

Intermediate 101

2-Methoxy-4-methyl-1-nitro-benzene

To a solution of 5-Methyl-2-nitrophenol (100.0 g, 0.65 mol) in dry acetone (2000 mL) was added K₂CO₃ (135.0 g, 1.5 eq.) and methyl iodide (405 mL, 10 eq.) and the mixture was stirred under reflux for 4 hours. The solution was filtered off and was evaporated off. The oil was diluted with DCM and washed with water. The organic layer was dried over Na₂SO₄ and evaporated to dryness to give the title compound (108.0 g, 0.65 mol) as a yellow oil.

¹H NMR (CDCl₃, 250 MHz) δ 7.65 (s, 1H), 7.25 (dd, 1H), 6.9 (d, 1H), 3.9 (s, 3H), 2.3 (s, 3H).

Ref: ex-Aldrich

Intermediate 102

2-Methoxy-4-methyl-phenylamine

To a solution of intermediate 101 (108.0 g, 0.65 mol) in EtOH (2000 mL) was added tin(II) chloride dihydrate (584.0 g, 4 eq.) and the mixture was stirred at 70° C. for 12 hours. The solution was evaporated off. The oil was diluted with DCM and washed with a NaOH solution (50%) and water. The organic layer was dried over Na₂SO₄ and evaporated to dryness to give the title compound (75.0 g, 0.55 mol) as a solid.

¹H NMR (CDCl₃, 250 MHz) δ 6.6 (m, 1H), 6.45 (m, 2H), 6.9 (d, 1H), 3.75 (s, 3H), 3.6 (bs, 2H), 2.15 (s, 3H).

Ref: WO 97-DK58 19970210.

Intermediate 103

1-(2-Methoxy-4-methyl-phenyl)-piperazine

To a solution of intermediate 102 (8.6 g, 64 mmol) in nBuOH (250 mL) was added Bis(2-chloroethyl)amine hydrochloride (12.6 g, 1.1 eq.). The resulting mixture was stirred under reflux for 27 hours. After cooling to rt, Na₂CO₃ (6.8 g, 1 eq.) was added and the mixture was stirred under reflux for 16 hours. The solution was filtered off and was evaporated off. The resulting precipitate was treated with water and basified with concentrated NaOH until pH=11. Extraction with DCM, drying over Na₂SO₄ and evaporating to dryness gave a residue which was flash chromatographed using DCM/IprNH₂ (95/5, and 85/15) to give the title compound (8.9 g, 43 mmol) as a pink oil.

GC/MS: M+ C₁₂H₁₈N₂O 206

Intermediate 104

2-{4-[4-(2-Ethoxymethyl-phenyl)-piperidin-1-yl]-butyl}-isoindole-1,3-dione

A solution of intermediate 103 (39.5 g, 0.18 mol) in acetone (600 mL) was treated with Cs₂CO₃ (64.5 g, 1.1 eq.) and N-(4-Bromobutyl)-phtalimide (50.9 g, 1.0 eq.). The resulting mixture was stirred under reflux for 24 hours. After cooling to rt the reaction mixture was filtered off. The cake was washed with acetone. The filtrate was evaporated off to give the title compound (60.0 g, 0.14 mol) as a yellow oil.

GC/MS: M+ C₂₆H₃₂N₂O₃ 420

Intermediate 105

4-[4(2-Ethoxy-4-methyl-phenyl)-piperidin-1-yl]-butylamine

A solution of intermediate 104 (60.0 g, 0.14 mol) in MeOH (600 mL) was treated with hydrazine hydrate (28 mL). The resulting mixture was stirred at 60° C. for 3 hours. After evaporation under reduced pressure the residue was taken up in water and treated with a concentrated HCl solution until PH=3. The white precipitate was filtered off, washed with water and the filtrate was treated with a concentrated NaOH solution until PH=13. Extraction with DCM, drying over Na₂SO₄ and filtration gave the title compound (37.0 g, 0.13 mol) as a yellow oil.

GC/MS: M+ C₁₈H₃₀N₂O 290

Intermediate 106

4-Nitro-4′-trifluoromethyl-biphenyl

To a solution of 4-(Trifluoromethyl)phenyl boronic acid (10.0 g, 52.7 mmol) in ethyleneglycol monomethyl ether (300 mL) was added 1-bromo-4-nitrobenzene (9.68 g, 47.9 mmol), a solution of NaHCO₃ (6.0 g) in water (35 mL) and tetrakis(triphenylphosphine)palladium (0) (1.0 g, 10% w/w). The resulting mixture was stirred to reflux for 18 hours and then after cooling, filtered on a bed of celite.

The solution was evaporated off and the residue treated with water and filtered off. The powder was then dissolved in Et₂O (200 mL), washed with water (2×100 mL). The organic phase was dried over Na₂SO₄, filtrated and evaporated off. The title compound was obtained as a brown powder (13.0 g, 48.7 mmol) in a 92% yield.

GC/MS: M+ C₁₃H₈F₃NO₂ 267

Intermediate 107

4′-Trifluoromethyl-biphenyl-4-yl-amine

To a solution of intermediate 106 (13.0 g, 48.7 mmol) in EtOH (500 mL) was added Pd/C (10% w/w, 1.3 g) and the mixture was shaken under a hydrogen atmosphere for 18 hours at rt.

The mixture was then filtered through a bed of celite and the solvent was evaporated in vacuo to give the title compound (11.2 g, 47.2 mmol) as a white powder in a 97% yield.

GC/MS: M+ C₁₃H₁₀F₃N 237

Intermediate 108

5-[4-(2-Ethoxy-4-methyl-phenyl)-piperidin-1-yl]-pentanoic Acid

A solution of Intermediate 11 (2.19 g, 10.0 mmol) and potassium carbonate (2.76 g, 2.0 eq.) in acetone (150 ml) was stirred at reflux for 30 min then ethyl bromovalerate (2.09 g, 1.1 eq.) was added and the mixture was stirred to reflux for 18 hours. After cooling, the reaction was filtered off and the solvent was evaporated in vacuo. The oil was dissolved in DCM and washed with water. The organic phase was dried over Na₂SO₄ and evaporated in vacuo. The residue was then dissolved in EtOH (500 mL) and stirred to reflux with a 1N NaOH solution (9.5 mL, 1.0 eq.) for 3 hours. After cooling, a 1N HCl solution was added (10 mL) and the solvent was evaporated in vacuo. The residue was triturated in MeOH to give, after filtration the title compound (3.08 g, 9.65 mmol) in a 96% yield.

GC/MS: M+ C₁₉H₂₉NO₃ 319

Intermediate 109

4′-(5-Bromo-pentoxy)-biphenyl-4-carbonitrile

To a solution of 4′-Hydroxy-biphenyl-4-carbonitrile (1.0 g, 5.12 mmol) in dry DMF (20 mL) was added NaH 60% (0.230 g, 1.2 eq.) and 1,5-bibromopropane (1.15 g, 1.0 eq.). The resulting mixture was stirred at rt for 12 hours and the solvent ˜was evaporated off. The residue was washed with water and extracted with DCM. The organic phase was dried over Na₂SO₄ and evaporated in vacuo. After purification by flash chromatography, the title compound was obtained as white crystals (0.72 g, 2.1 mmol) in a 42% yield.

¹H NMR (CDCl₃, 300 MHz) δ 7.7 (m, 4H), 7.6 (d, 2H), 7 (d, 2H), 4.1 (m, 2H), 1.9 (m, 4H), 1.7 (m, 4H).

Intermediate 110

1-[4-(1−Hydroxy-naphtalen-2-yl)-3,6-dihydro-2H-pyridin-1-yl]-ethanone

The same method was employed as in the preparation of intermediate 8 but starting from the 1-Naphtol gave the title compound as a white solid in a 54% yield.

GC/MS: M+ C₁₇H₁₇NO₂ 267

Intermediate 111

1-[4-(1−Hydroxy-naphtalen-2-yl)-piperidin-1-yl]-ethanone

A solution of intermediate 110 (29.0 g, 0.112 mol) in a mixture of cyclohexene (450 mL), MeOH (100 mL), THF (350 mL) was treated with Pd(OH)₂, 50% (14 g). The resulting solution was allowed to stir at reflux for 4 days. After cooling, the reaction mixture was filtered through a bed of celite. The filtrate was evaporated to dryness to give the title compound as a white solid (22.0 g, 0.082 mol) in a 73% yield after recrystallization from CH₃CN.

LC/MS: [M+H⁺] C₁₇H₁₉NO₂ 270

Intermediate 112

1-[4-(1−Methoxy-naphtalen-2-yl)-piperidin-1-yl]-ethanone

To a solution of intermediate 111 (22.0 g, 0.08 mol) in dry DMF (400 mL) was added K₂CO₃ (23.0 g, 2 eq.) and methyl iodide (20.4 mL, 4 eq.). The reaction was stirred at 80° C. for 16 hours. After cooling, the reaction was filtered off and evaporated under reduced pressure. The oil was diluted with DCM and washed with water. The organic layer was dried over Na₂SO₄ and evaporated to dryness to give the title compound as a white solid in a quantitative yield.

GC/MS: M+ C₁₈H₂₁NO₂ 283

Intermediate 113

4-(1-Methoxy-naphtalen-2-yl)-piperidine

To a solution of the intermediate 112 (23.0 g, 82 mmol) in EtOH (400 mL) was added dropwise a 1/1 solution of a concentrated NaOH solution and H₂O (100 mL). The resulting mixture was stirred at 100° C. during 16 hours. After cooling to rt and evaporation under reduced pressure, the residue was taken up in DCM and washed with water. The organic phase was dried over Na₂SO₄ and evaporated off to give the title compound as an oil (10.6 g, 44 mmol).

GC/MS: M+ C₁₆H₁₉NO 241

Intermediate 114

4′-(4-Chloro-butoxy)-biphenyl-4-carbonitrile

The same method was employed as in the preparation of intermediate 109 but starting from 1-bromo-4-chlorobutane gave the title compound as white crystals in a 44% yield.

¹H NMR (CDCl₃, 300 MHz) δ 7.9 (m, 4H), 7.7 (d, 2H), 7.2 (d, 2H), 4.25 (t, 2H), 3.8 (t, 2H), 2.2 (m, 4H).

Intermediate 115

4-Amino-3-iodo-benzoic Acid Methyl Ester

Ref: tet.lett. 1997, 38, 2307

To a solution of Methyl 4-aminobenzoate (17.0 g, 0.1 mol) in DCM (200 mL) was added at rt, benzyltrimethylammonium dichloroiodate (40.0 g, 1.02 eq.). The solution was stirred under a nitrogen atmosphere for 24 hours and a turbid mixture was finally obtained. After filtration of the mixture, the filtrate was washed with a saturated solution of NaHCO₃ and brine. The organic phase was dried over Na₂SO₄ and concentrated under vacuo to give the title compound (25.0 g, 0.09 mol) as a colorless powder in a 90% yield

MP: 135-139° C.

GC/MS: M+ C₈H₈₁NO₂ 277

Intermediate 116

4-Acetylamino-3-iodo-benzoic Acid Methyl Ester

To a solution of intermediate 115 (25.0 g, 90 mmol.) in DCM (200 mL) was added DMAP (0.11 g, 0.01 eq.) and TEA (38 mL, 3 eq). Acetyl chloride (19.2 mL, 3 eq.) was added dropwise and the mixture was stirred 3 hours at rt and concentrated under vacuo. The brown powder obtained was triturated with a mixture of Et₂O (200 mL) and CH₃CN (5 mL) to give after filtration the title compound (19.0 g, 60 mmol.) in a 67% yield.

MP: 140-141° C.

Intermediate 117

2-(4-Chloro-phenyl)-1H-indole-5-carboxylic Acid Methyl Ester

To a solution of intermediate 116 (3.19 g, 10 mmol) in dioxane (150 mL) and THF (150 mL) was added 1-chloro-4-ethynylbenzene (1.64 g, 1.2 eq.). Then, dropwise, was added tetramethylguanidine (25 mL, 20.0 eq.), bis(triphenylphosphine)palladium (II) chloride (0.7 g, 0.1 eq.) and copper iodide (0.19 g, 0.1 eq.). The resulting mixture was heated at 80° C. for 48 hours. After cooling, the mixture was filtered and concentrated under vacuo. The residue was triturated with iPr₂O and the precipitate obtained was filtered, washed 3 times with water to give the title compound (0.6 g, 2.0 mmol.) as a white powder in a 20% yield.

MP: Decomposition above 254° C.

LC/MS (APCI): [M−H⁺] C₁₆H₁₂ClNO₂ 284

Intermediate 118

2-(4-Chloro-phenyl)-1-methyl-1H-indole-5-carboxylic Acid Methyl Ester

To a solution of intermediate 117 (1.43 g, 5 mmol.) in dry THF (100 mL) was added NaH (0.14 g, 1.15 eq.). After stirring for 40 minutes at rt, the mixture was cooled to 0° C. and methyl iodide (0.78 g, 1.1 eq.) in dry THF (5 mL) was added dropwise. The mixture was strirred at rt for 3 hours and quenched with water. The THF was removed under vacuo and the aqueous phase was extracted two times with DCM (150 mL). The organic phase was dried over Na₂SO₄, filtrated, evaporated off and flash chromatographed using Chex//EtOAc (90/10) to give the title compound (1.1 g, 3.7 mmol.) as a white powder in a 74% yield.

LC/MS (APCI): [M+H⁺] C₁₇H₁₄ClNO₂ 300

Intermediate 119

2-(4-Chloro-phenyl)-1-methyl-1H-indole-5-carboxylic Acid

A solution of 1N NaOH (40 mL, 12 eq.) was added to a solution of intermediate 118 (0.98 g, 3.3 mmol.) in EtOH (125 mL). The mixture was heated for 18 hours at 70° C. and after complete consumption of the starting material the reaction was concentrated under vacuo. The powder was triturated with water and sonicated. The precipitate was then filtrated and dried under vacuo to give the title compound (0.8 g, 2.8 mmol.) as a white powder in a 85% yield.

MP>260° C.

LC/MS (APCI): [M−H+] C₁₆H₁₂ClNO₂ 284

Intermediate 120

2-(4-Trifluoromethyl-phenyl)-benzofuran-5-carboxylic Acid Methyl Ester

Ref: Synthesis, 1992, 3, 293

A mixture of 3-Formyl-4-hydroxy-benzoic acid methyl ester (1.8 g, 10 mmol.), 4-trifluoromethylbenzyl-bromide (2.39 g, 1 eq.) and potassium carbonate (4.15 g, 3 eq.) in DMF (75 mL) was heated at 160° C. for 4 hours. After cooling to rt, the reaction mixture was filtered off. The cake was washed with DMF. The filtrate was evaporated off and the residue was triturated with MeOH. After filtration, the title compound (1.34 g, 4.2 mmol.) was obtained of the as a white powder in a 42% yield.

MP: 180° C.

GC/MS: M+ C₁₇H₁₁F₃O₃ 320

Intermediate 121

2-(4-Trifluoromethyl-phenyl)-benzofuran-5-carboxylic Acid

A mixture of intermediate 120 (1.25 g, 3.9 mmol.) in EtOH (20 mL) and a 1N NaOH solution (39 mL, 10 eq.) was heated at 70° C. for 1.30 hours. After cooling to rt, a 1N HCl solution (78 mL) was added and the resulting white suspension was filtered off and washed with water to give the title compound (1.17 g, 3.8 mmol.) as a white powder in a 98% yield.

MP>260° C.

LC/MS (APCI): [M−H+] 305 C₁₆H₉F₃O₃

Intermediate 122

4-Hydroxy-3-iodo-benzoic Acid Ethyl Ester

HCl gas was bubbled in 3-Iodo-4-hydroxybenzoic acid (3.0 g, 11.4 mmol.) in solution in EtOH (200 mL). The mixture was stirred at rt for 96 hours. After concentration under vacuo, the green crude product obtained was flash chromatographed using DCM/MeOH (96/4) as eluent to give the title compound (2.73 g, 9.3 mmol.) as a white powder in a 82% yield.

MP: 114° C.

LC/IMS (APCI): [M−H+] 291 C₉H₉IO₃

Intermediate 123

2-(4-Chlorophenyl)-benzofuran-5-carboxylic Acid Methyl Ester

To a solution of intermediate 122 (1.46 g, 5 mmol.) and 1-chloro-4-ethynylbenzene (0.82 g, 1.2 eq.) in DMF (30 mL) was added dropwise tetramethylguanidine (6.26 mL, 10 eq.), bis(triphenylphosphine)palladium (II) chloride (0.35 g, 0.1 eq.) and copper iodide (0.095 g, 0.1 eq.). The resulting mixture was heated at rt for 24 hours then filtered off and the filtrate was concentrated under vacuo. The residue was flash chromatographed using Chex/EtOAc (92/8) to give the title compound (0.425 g, 1.4 mmol.) as white crystals in a 28% yield.

¹H NMR (d₆ DMSO, 300 MHz) δ 8.4 (s, 1H), 8.1 (dd, 3H), 7.9 (dd, 1H), 7.8 (dd, 3H), 4.4 (q, 2H), 1.4 (q, 3H).

Intermediate 124

2-(4-Chloro-phenyl)-benzofuran-5-carboxylic acid The same method was employed as in the preparation of intermediate 121 but starting from intermediate 123 gave the title compound as white powder in a 72% yield.

¹H NMR (d₆DMSO, 300 MHz) δ 13.2 (s, 1H), 8.4 (s, 1H), 8.2 (dd, 3H), 7.9 (dd, 1H), 7.8 (dd, 3H)_{z},

Intermediate 125

2-(3,4-Dichloro-phenyl)-benzofuran-5-carboxylic Acid

A mixture of 3-Formyl-4-hydroxy-benzoic acid methyl ester (2.7 g, 15 mmol.), 3,4-dichlorobenzylbromide (3.6 g, 1 eq.) and (6.22 g, 3 eq.) of potassium carbonate in DMF (75 mL) was heated at 170° C. for 24 hours. The crude product obtained after concentration under vacuo was diluted with DCM (300 mL) and washed with water. The organic phase and the white suspension were mixed and concentrated under vacuo. The residue was then recrystallized in a 1:1 EtOH/H₂O mixture. The title compound (1.38 g, 4.5 mmol.) was obtained as a colorless powder in a 30% yield.

MP>260° C.

LC/IMS (APCI): [M−H+] 306 C₁₅H₈Cl₂O₃

Intermediate 126

1-[4-(1−Hydroxy-5,6,7,8-tetrahydro-naphtalen-2-yl)-3,6-dihydro-2H-pyridin-1-yl]-ethanone

The same method was employed as in the preparation of intermediate 8 but starting from the 5,6,7,8-tetrahydro-1-naphtol to give the title compound as a powder after crystallization in CH₃CN in a 100% yield.

GC/MS: M+ C₁₇H₂₁NO₂ 271

Intermediate 127

1-[4-(1−Hydroxy-5,6,7,8-tetrahydro-naphtalen-2-yl)-piperidin-1-yl]-ethanone

To a solution of intermediate 126 (55.0 g, 0.203 mol) in AcOH (500 mL) was added Pd/C, 10% (2 g) and the reaction was stirred under an atmospheric pressure of hydrogen at 50° C. for 24 hours. The mixture was filtered through a bed of celite. The filtrate was evaporated under reduced pressure to give the title compound (55.0 g, 0.201 mol) as a yellow powder.

GC/MS: M+ C₁₇H₂₂NO₂ 273

Intermediate 128

1-[4-(1−Cyclopropylmethoxy-5,6,7,8-tetrahydro-naphtalen-2-yl)-piperidin-1-yl]ethanone

To a solution of intermediate 127 (11.2 g, 0.041 mol) in dry acetone and DMF (200 mL, 1/1) was added Cs₂CO₃ (20.05 g, 1.5 eq.) and Bromomethylcyclopropane (6.09 g, 1.1 eq.). The reaction was stirred at 55° C. for 13 hours. After cooling, the reaction was filtered off and washed with acetone. The filtrate was evaporated under reduced pressure to give the title compound as an yellow oil in a quantitative yield. The crude product was used in the next step without purification.

Intermediate 129

4-(1-Cyclopropylmethoxy-5,6,7,8-tetrahydro-naphtalen-2-yl)-piperidine

The same method was employed as in the preparation of intermediate 11 but starting from the intermediate 128 to, give the title compound as an oil in a 90% yield.

¹H NMR (CDCl₃, 300 MHz) δ 6.95 (d, 1H), 6.8 (d, 1H), 3.5 (m, 2H), 2.9 (m, 2H), 2.8 (m, 4H), 2.3 (m, 2H), 1.9 (m, 2H), 1.8 (m, 4H), 1.4 (m, 4H), 1.1 (m, 1H), 0.45 (m, 2H), 0.25 (m, 2H).

Intermediate 130

2-{4-[4-(1−Cyclopropylmethoxy-5,6,7,8-tetrahydro-naphtalen-2-yl)-piperidin-1-yl]butyl}-isoindole-1,3-dione

The same method was employed as in the preparation of intermediate 6 but starting from the intermediate 129 to give after flash chromatography using (DCM/MeOH, 95/5 and 90/10) as eluent, the title compound as an orange oil in a 80% yield.

¹HNMR (CDCl₃, 300 MHz) δ 7.8 (m, 2H), 7.6 (m, 2H), 6.9 (d, 1H), 6.7 (d, 1H), 3;7 (m, 2H), 3.4 (m, 2H), 3.0 (m, 2H), 2.6 (m, 4H), 2.4 (m, 2H), 1.9 (m, 2H), 1.7 (m, 13H), 1-0.1 (m, 1H), 0.45 (m, 2H), 0.25 (m, 2H).

Intermediate 131

4-[4-(1−Cyclopropylmethoxy-5,6,7,8-tetrahydro-naphtalen-2-yl)-piperidin-1-yl]-butylamine

The same method was employed as in the preparation of intermediate 7 but starting from intermediate 130 gave the title compound as an orange oil in a 90% yield.

LC/MS (APCI): [M+H⁺] 357 C₂₃H₃₆N₂O

Intermediate 132

2-(6-Trifluoromethyl-pyridin-3-yl)-benzofuran-5-carboxylic Acid Methyl Ester

To a solution of 3-Formyl-4-hydroxy-benzoic acid methyl ester (5.0 g, 0.03 mol) in dry DMF (80 mL) was added potassium carbonate (12.42 g, 3.0 eq.) and 5-Bromomethyl-2-trifluoromethyl-pyridine (6.7 g, 1.0 eq.) The resulting mixture was stirred at 160° C. for 18 hours. The solution was evaporated off and the resulting precipitate was treated with water and filtered off to give the title compound (7.42 g, 23 mmol) as white crystals after washed with hot MeOH in a 77% yield.

GC/MS: M+ C₁₆H₁₀F₃NO₃ 321

Intermediate 133

2-(6-Trifluoromethyl-pyridin-3-yl)-benzofuran-5-carboxylic Acid

A solution of intermediate 132 (7.42 g, 0.023 mol) in MeOH/EtOH (100 mL/10 mL) was treated with a 1N NaOH solution (115 mL, 5 eq.) and the resulting mixture was stirred at reflux for 4 hours. After cooling to rt., a 1N HCl solution (115 mL, 5 eq.) was added and the solvent was evaporated off. The residue was treated with water to give after filtration and dry, the title compound (4.44 g, 15 mmol) as white crystals in a 63% yield.

MP>260° C.

GC/MS: M+ C₁₅H₈F₃NO₃ 307

Intermediate 134

4-(Diethoxy-phosphoylmethyl)-benzoic Acid Methyl Ester

To methyl 4-(bromomethyl)benzoate (23.0 g, 0.1 mol.) was added triethylphosphite (30 mL, 1.7 eq.).

The resulting mixture was stirred at 135° C. for 18 hours. The crude solution was then distilled under reduced pressure (170-180° C., 15 mm/Hg) and the title compound (19.4 g, 67.8 mmol) was obtained as a colourless oil in a 68% yield.

GC/MS: M+ C₁₃H₁₉PO₅ 286

Intermediate 135

4-[2-(4-Trifluoromethyl-phenyl)-vinyl]-benzoic Acid Methyl Ester

To a solution of intermediate 134 (11.6 g, 40 mmol) in dry THF (200 mL) under argon, was added sodium hydride 60% (1.6 g, 1.0 eq.). The resulting mixture was stirred 30 min at rt.

Then a solution of 4-(trifluoromethyl)benzaldehyde (6.96 g, 1.0 eq.) in dry THF (20 mL) was added and the mixture was stirred 1 hour at rt. After filtration, the solvent was evaporated off. The white solid obtained was recrystallized from EtOH (50 mL) and the crystals was washed with diisopropyl ether to give the title compound (3.4 g, 11 mmol) in a 28% yield.

GC/MS: M+ C₁₇H₁₃F₃O₂ 306

Intermediate 136

4-[2-(4-Trifluoromethyl-phenyl)-vinyl]-benzoic Acid

To a solution of intermediate 135 (3.4 g, 11 mmol) in EtOH (100 mL) was added a 1N NaOH solution (30 mL). The mixture was stirred at reflux for 1 hour. After cooling to rt, a 1N HCl solution (30 mL) was added to give white precipitate which was filtered off and washed with water to give the title compound (3.05 g, 10.4 mmol) in a 94% yield.

¹H NMR (d⁶ DMSO, 300 MHz) δ 13.0 (bs, 1H), 8.0 (d, 2H), 7.85 (d, 2H), 7.75 (m, 4H), 7.5 (bs, 2H).

Intermediate 137

4-(4-Trifluoromethyl-benzyloxy)-benzoic Acid Methyl Ester

To a solution of Ethyl 4-hydroxybenzoate (8.0 g, 0.048 mol.) in acetone was added Cs₂CO₃ (17.27 g, 1.1 eq.) and 4-(Trifluoromethyl)benzyl bromide (10.0 g, 1.0 eq.). The resulting mixture was stirred to reflux for 3 hours. The crude solution was filtered off and the solvent was evaporated under reduced pressure to give the title compound (13.0 g, 0.04 mmol.) as a white powder in a 96% yield.

GC/MS: M+ C₁₇H₁₅F₃O₃ 324

Intermediate 138

4-(4-Trifluoromethyl-benzyloxy)-benzoic Acid

A mixture of intermediate 137 (13.0 g, 0.04 mol.) in EtOH (300 mL) and a 1N NaOH solution (46 mL) was stirred to reflux for 2 hours. After cooling to rt, a 1N HCl solution (46 mL) was added and the resulting white suspension was filtered off and washed with water to give the title compound (10.0 g, 0.033 mol.) as a white powder in a 82.5% yield.

LC/MS (APCI): [M+H⁺] 297 C₁₅H₁₁F₃O₃

Intermediate 139

4-[2-(3,5-Dichloro-phenyl)-vinyl]-benzoic Acid Methyl Ester

A solution of intermediate 134 (8.17 g, 28.6 mmol) in dry THF (100 mL) was treated with NaH (60% in dispersion in oil, 1.1 eq.) for 1 hour at rt. A solution of 3,5-Dichloro-benzaldehyde (5.0 g, 28.6 mmol) in THF (30 mL) was added and the resulting mixture was stirred at 40° C. for 1 hour. After filtration, the filtrate was evaporated under reduced pressure to give the title compound (8.75 g 28.5 mmol) as white crystals after recriztallization from EtOH in a 99% of yield.

GC/MS: M+ C₁₆H₁₂Cl₂O₂ 308

¹H NMR (CDCl₃, 250 MHz) δ 8.0 (d, 2H), 7.4 (d, 2H), 7.3 (d, 2H), 7.15 (d, 1H), 7.0 (d, 2H), 3.7 (s, 3H).

Intermediate 140

4-[2-(3,5-Dichloro-phenyl)-vinyl]-benzoic Acid

To a solution of intermediate 139 (8.75 g, 28.5 mmol) in MeOH (100 mL) was added a 1N NaOH solution (43 mL, 1.5 eq.). The reaction was stirred under reflux for 12 hours. After cooling, a 1N HCl solution (1 eq.) was added. A precipitate was formed. After filtration, the cake was washed with H₂O and dried to give the title compound (5.0 g, 17.0 mmol) as white solid after recristallization from MeOH in 60% yield.

MP: 273° C.

Intermediate 141

5-ethoxy-2-(hydroxy-pyridin-4-yl-methyl)-phenol

To a solution of 3-ethoxy-phenol (12.7 g, 0.092 mol) and 4-pyridincarboxaldehyde (9.84 g, 0.092 mol) in dry DCM (500 mL) was added a solution of TiCl4 (11 mL, 0.101 mol, 1.1 eq) in DCM (50 mL) at −50° C. for 50 min. The mixture was stirred at rt for 1.5 hours and then was pourred into crushed ice (200 g). The pH was adjusted at 7.5-8 to give a yellow solid which was filtered off. The solid was washed with THF and the organic phase was dried over Na₂SO₄ and then evaporated off. The residue was triturated with Et₂O and MeOH to give the title compound as a white solid (7.4 g, 0.03 mol) in a 33% yield.

LC/MS (APCI): [M+H+] 246 C₁₄H₁₆NO₃

Intermediate 142

5-ethoxy-2-piperidin-4-yl-methyl-phenol

To a solution of intermediate 141 (7.4 g, 0.03 mol) in MeOH/HCl 1N (1/1) (200 mL) was added Pd/C 10% (0.6 g). The mixture was stirred at 30° C. for 24 hours under a hydrogen atmosphere.

The mixture was filtered off on celite and evaporated off to give the title compound (4 g, 0.017 mol), as a brown solid in a 56% yield.

GC/MS: M+ 235 C₁₄H₂₁NO₂

Intermediate 143

2-{4-[4-(4-ethoxy-2-hydroxy-benzyl)-piperidin-1-yl]-butyl}-isoindole-1,3-dione

The same method was employed as in the preparation of intermediate 6 but starting from intermediate 142 gave the title compound as a brown solid (1.3 g, 3 mmol) after purification by flash chromatography using DCM/MeOH(95/5) as eluent in a 18% yield.

LC/MS (APCI): [M+H+] C₂₆H₃₃N₂O₄ 437

Intermediate 144

2-{4-[4-(2,4-diethoxy-benzyl)-piperidin-1-yl]-butyl}-isoindole-1,3-dione

To a solution of intermediate 143 (0.680 g, 1.56 mmol) in dry DMF was added CSOH monohydrate (0.262 g, 1.56 mmol). The mixture was stirred 1 hour at rt. A solution of Ethyl Iodide (0.390 g, 2.5 mmol, 1.6 eq) in dry DMF was added dropwise, then the mixture was stirred at rt for 48 hours. The mixture was filtered off and the solvent was removed in vacuo. After purification by flash chromatography, using DCM/MeOH (9/1) as eluent, the title compound was obtained as a brown oil (0.310 g, 0.67 mmol) in a 43% yield.

GC/MS: M+ 464 C₂₈H₃₆N₂O₄

Intermediate 145

4-[4-(2,4-diethoxy-benzyl)-piperidin-1-yl]-butylamine

The same method was employed as in the preparation of intermediate 7 but starting from intermediate 144 gave the title compound as a yellow oil (0.21 g, 0.63 mmol) in a 88% yield.

GC/MS: M+ C₂₀H₃₄N₂O₂ 334

Intermediate 146

1-[4-(2,4-dimethoxy-benzoyl)-piperidin-1-yl]-ethanone

To a solution of m-dimethoxy-benzene (30.2 g, 0.219 mol) in dry DCM was added a solution of pure TiCl₄ (60 mL, 0.549 mol, 3 eq) in dry DCM (150 mL) at −78° C. for 50 min. Then a solution of 1-acetyl-piperidine-4-carbonyl chloride (34.6 g, 0.183 mol) in dry DCM (300 mL) was added at −78° C. The mixture was stirred at rt for 18 hours and then was pourred in crushed ice (500 g). NH₄Cl (200 ml) satured solution was added. The mixture was treated with a 1N HCl solution, and then extracted with DCM. The organic phase was dried over Na₂SO₄ and evaporated off to give an orange solid which became white upon addition of a 1N HCl solution (20 mL). The solid was filtered off and washed with diisopropyl ether to give the title compound (30 g, 0.103 mol) as a white solid in a 56% yield.

GC/MS: M+ 291C₁₆H₂₁NO₄

Intermediate 147

(2,4-dimethoxy-phenyl)-piperidinyl-methanone

To a solution of intermediate 146 (18.9 g, 65 mmol) in MeOH (200 mL) was added a concentrated NaOH solution/H₂O (1/1) solution (130 mL) and the reaction was stirred to reflux for 24 hours. After cooling, the reaction was concentrated in vacuo, and the residue was diluted with water and extracted with DCM. The organic phase was washed with brine an rater, extracted off and then dried over Na₂SO₄ and evaporated off.

The title compound was obtained as a yellow oil (14.3 g, 57.4 mmol) in a 88% yield.

GC/MS: M+ 249 C₁₄H₁₉NO₃

Intermediate 148

2-(1-Benzyl-1,2,3,6-tetrahydro-pyridin-4-yl)-5-methyl-phenol

To a solution of m-Cresol (20.0 g, 0.185 mol) and 1-Benzyl-4-piperidone (35.0 g, 1.0 eq.) was added dropwise BF₃-Et₂O (71 mL, 3.0 eq). The mixture was stirred at 100° C. for 24 hours. After cooling to rt, the mixture was treated with a 1N HCl solution (400 mL). The resulting solution was extracted with DCM. The organic layer was dried over Na₂SO₄ and evaporated to dryness to give an oil which was crytallized in cyclohexane to give the title compound (40.0 g, 0.14 mol) as a yellow powder.

GC/MS: M+ C₁₉H₂₁NO 279

Intermediate 149

5-Methyl-2-piperidin-4-yl-phenol

To a solution of intermediate 148 (40.0 g, 0.14 mol) in EtOH (600 mL) and THF (50 mL) was added Pd/C, 10% (4.0 g) and the reaction was stirred under an atmospheric pressure of hydrogen at 50° C. for 56 hours. The reaction mixture was filtered through a bed of celite. The filtrate was evaporated under reduced pressure to give the title compound as a white powder in a quantitative yield.

GC/MS: M+ C₁₂H₁₇NO 191

Intermediate 150

2-(1−Benzyl-1,2,3,6-tetrahydro-pyridin-4-yl)-5-ethyl-phenol

A solution of 3-Ethyl-phenol (6.1 g, 0.05 mol) and 1-Benzyl-4-piperidone (10.0 g 1.05 eq.) in acetic acid (100 mL) was treated with HCl gaz for 10 min. The mixture was stirred at 95° C. for 30 min. After cooling to rt, the mixture was treated again with HCl gaz for 5 min. The resulting solution was allowed to stir at rt for 4 days. The solvent was evaporated under reduced pressure and the residue was diluted with H₂O and extracted with DCM. The organic layer was washed with a 2N NaOH solution, H₂O and brine, dried over Na₂SO₄ and evaporated to dryness. The residue was flash chromatographed using MeOH/DCM (5/95) to give the title compound (8.0 g, 0.027 mol) as a yellow oil in 54% yield.

GC/MS: M+ C₂₀H₂₃NO 293

Intermediate 151

5-Ethyl-2-piperidin-4-yl-phenol

To a solution of intermediate-150 (8.0 g, 0.027 mol) in EtOH (100 mL) was added Pd/C, 10% (0.8 g) and the reaction was stirred under an atmospheric pressure of hydrogen for 24 hours. The reaction mixture was filtered through a bed of celite. The filtrate was evaporated under reduced pressure to give the title compound (4.9 g, 0.024 mol) as a yellow oil in a 88% yield.

GC/MS: M+ C₁₃H₁₉NO 205

Intermediate 152

2-Piperidin-4-yl-5,6,7,8-tetrahydro-naphtalen-1-ol

To a solution of intermediate 127 (27.0 g, 0.099 mol) in EtOH (750 mL) was added a solution of NaOH (250 mL) in H₂O (250 mL). The reaction was stirred under reflux for 16 hours. After cooling, the reaction was concentrated under reduced pressure, was diluted with DCM and washed with water. The organic layer was dried over Na₂SO₄ and evaporated to dryness to give after flash chromatography using DCM/MeOH/NH₄OH 30,30,30 as eluent, the title compound (9.7 g, 0.042 mol) as a pink gummy oil in a 42.5% yield.

¹H NMR (CDCl₃, 300 MHz) δ 7.9 (bs, 1H), 6.8 (d, 1H), 6.6 (d, 1H), 3.4 (m, 2H), 3.1 (m, 2H), 2.8 (m, 4H), 1.8-1.4 (m, 10H.

Intermediate 153

2-{4-[4-(1−Hydroxy-5,6,7,8-tetrahydro-naphtalen-2-yl)-piperidin-1-yl]-butyl}-isoindole-1,3-dione

The same method was employed as in the preparation of intermediate 77 but starting from the intermediates 152 and 76 to give after flash chromatography using (DCM/MeOH, 90/10 and 1% ammoniac solution) as eluent, the title compound as a gummy oil in a 46% yield.

¹H NMR (CDCl₃, 300 MHz) δ 7.9 (m, 2H), 7.75 (m, 2H), 6.9 (d, 1H), 6.8 (d, 1H), 6.4 (bs, 1H), 3.85 (m, 2H), 3.5 (m, 2H), 3.0 (m, 1H), 2.9 (m, 2H), 2.8 (m, 2H), 2.5 (m, 4H), 2.1 (m, 2H), 1.87 (m, 10H). WO

Intermediate 154

2-[1−(4-Amino-butyl)-piperidin-4-yl]-5,6,7,8-tetrahydro-naphtalen-1-ol

The same method was employed as in the preparation of intermediate 7 but starting from intermediate 153 to give the title compound as a red oil in a 90% yield.

¹H NMR (CDCl₃, 300 MHz) &7.0 (d, 1H), 6.6 (d, 1H), 3.1 (m, 2H), 2.9 (m, 1H), 2.65 (m, 4H), 2.6 (m, 2H), 2.45 (m, 2H), 2.1 (m, 2H), 1.85 (m, 8H), 1.5 (m, 6H).

Intermediate 155

2-Piperidin-4-yl-naphtalen-1-ol

The same method was employed as in the preparation of intermediate 152 but starting from the intermediate 111 gave the title compound as a brown solid in a quantitative yield.

¹H NMR (DMSO, d⁶, 300 MHz) δ 9.3 (s 1H), 8.25-(dd, 1H), 7.8 (dd, 1H), 7.5 (m, 3H), 7.25 (m, 1H), 3.45 (m, 3H), 3.1 (m, 2H), 2.9 (m, 4H).

Intermediate 156

2-{4-[4-(1−Hydroxy-naphtalen-2-yl)-piperidin-1-yl]-butyl}-isoindole-1,3-dione

The same method was employed as in the preparation of intermediate 153 but starting from the intermediate 155 gave the title compound as a pink solid in a 61% yield.

¹H NMR (CDCl₃, 300 MHz) δ 8.3 (dd, 2H), 7.95 (m, 2H), 7.8 (m, 3H), 7.6-7.2 (m, 4H), 3.85 (m, 2H), 3.25 (m, 2H), 2.85 (m, 2H), 2.55 (m, 2H), 2.35 (m, 2H), 1.95 (m, 2H), 1.8 (m, 4H).

Intermediate 157

2-1-(4-Amino-butyl)-piperidin-4-yl]-naphtalen-1-ol

The same method was employed as in the preparation of intermediate 7 but starting from intermediate 156 to give the title compound as a yellow solid in a 79% yield.

LC/MS(ES): M+ C₁₉H₂₆N₂O 298

### EXAMPLE 1

4-(4-chloro-benzoylamino)-N-{4-[4-(2,4-dimethoxy-phenyl)-piperidin-1-yl]-butyl}-benzamide Hydrochloride

A solution of intermediate 7 (3.58 g, 12 mmol) in DMF was treated with intermediate 2 (3.7 g, 1.1 eq.), EDCI (2.63 g, 1.1 eq.), HOBt (1.8 g, 1.1 eq.) and TEA (2 mL, 1.1 eq.). The resulting mixture was stirred for 16 hours at rt. The solvent was evaporated off. The residue was taken up in DCM and washed with a 1N NaOH solution and brine. The organic layer was dried over Na₂SO₄ and evaporated off. The residue was flash chromatographed using MeOH/DCM (10/90). Recrystallization from MeOH gave the title compound as white crystals in a 20% yield.

MP: 238° C.

Analysis for C₃₁H₃₆ClN₃O₄.HCl:

Calculated: C, 63.48; H, 6.36; N, 0.16. Found: C, 63.14; H, 6.51; N, 7.05.

### EXAMPLE 2

4-(4-chloro-benzoylamino)-N-{4-[4-(2-ethoxy-4-methyl-phenyl)-piperidin-1-yl]-butyl}-benzamide

To a solution of intermediate 13 (7.07 g, 0.02 mol) in dry THF (100 mL) and MeOH (175 mL) was added the intermediate 11 (5.0 g, 0.022 mol). The reaction was stirred at rt for 30 min and AcOH (1.5 eq) was added. Then sodium borohydride (1.2 eq.) was added and the reaction was stirred for 24 hours at rt and 7 hours to reflux. After cooling the solvent was evaporated and H₂O was added. The precipitate was filtered off and dried to give the title compound (8.1 g, 0.014 mol) as a white solid after washed in hot MeCN in 70% yield.

MP: 254° C.

LC/MS (APCI): [M+H⁺] C₃₂H₃₈N₃O₃Cl 548

### EXAMPLE 2A

4-(4-chloro-benzoylamino)-N-{4-[4-(2-ethoxy-4-methyl-phenyl)-piperidin-1-yl]-butyl}-benzamide Mesylate

A suspension of Example 2 (4.2 g) was heated to reflux in EtOH (100 mL). Then CH₃SO₃H (1 mL) was added. After filtration, the solution was cooled and leaved during 3 hours at rt. After total crystallization, the crystals was filtered and washed with cold EtOH. White crystals of title salt were obtained (3.4 g) in a 69.4% yield

MP: 210° C.

### EXAMPLE 3

4-(4-chloro-benzoylamino)-N-{4-[4-(2-ethoxy-4-ethyl-phenyl)-piperidin-1-yl]-butyl}-benzamide;

The same method was employed as in the preparation of Example 1 but starting from Intermediate 21 gave the title compound as crystals after crystallization from DMF in a 52% yield.

MP: 250° C.

Analysis for C₃₃H₄₀ClN₃O₃ (0.3, DMF)

Calculated: C, 69.71; H. 7.26; N, 7.91. Found: C, 69.56; H, 7.37; N, 7.7

### EXAMPLE 4

4-(4-chloro-benzoylamino)-N-{4-[4-(4-ethyl-2-methoxy-phenyl)-piperidin-1-yl]-butyl}-benzamide

The same method was employed as in the preparation of Example 1 but starting from Intermediate 25 gave the title compound as crystals after crystallization from DMF/EtOH in a 53% yield.

MP: 235° C.

Analysis for C₃₂H₃₈ClN₃O₃

Calculated: C, 70.12; H, 6.99; N. 7.67. Found: C, 70.24; H, 6.64; N, 7.64

### EXAMPLE 5

4-(4-chloro-benzoylamino)-N-{4-[4-(4-isopropyl-2-methoxy-phenyl)-piperidin-1-yl]-butyl}-benzamide

The same method was employed as in the preparation of Example 1 but starting from Intermediate 3.3 gave the title compound as crystals in a 46% yield.

MP: 241° C.

Analysis for C₃₃H₄₀ClN₃O₃ (0.5H₂O)

Calculated: C, 69.4; H, 7.24; N, 7.36. Found: C, 69.39; H, 7.55; N, 7.43

### EXAMPLE 6

4-(4-chloro-benzoylamino)-N-{4-[4-(2-ethoxy-4-isopropyl-phenyl)-piperidin-1-yl]-butyl}-benzamide

The same method was employed as in the preparation of Example 1 but starting from Intermediate 37 gave the title compound as crystals in a 43% yield.

MP: 242° C.

Analysis for C₃₄H₄₂ClN₃O₃ (0.5H₂O)

Calculated: C, 69.78; H, 7.41; N, 7.18. Found: C, 69.91; H. 7.45; N, 7.16

### EXAMPLE 7

4′-Trifluoromethyl-biphednyl-4-carboxylic Acid (4-{4-12,5-dimethyl-4-(pyridin-2-ylmethoxy)-phenyl]-piperidin-1-yl}-butyl)-amide

The same method was-employed as in the preparation of example 1 but starting from intermediate 46 and intermediate 36 gave the title compound as white crystals after recrystallization from EtOH in a 57% yield.

MP: 226° C.

Analysis for C₃₇H₄₀F₃N₃O₂, (0.2H₂O):

Calculated: C, 71.75; H. 6.57; N, 6.78. Found: C, 71.53; H. 6.22; N, 6.88

### EXAMPLE 8

4-(4-chloro-benzoylamino)-N-[4-(4-benzo[1,3]dioxol-5-yl-piperidin-1-yl]-butyl}-benzamide;

The same method was employed as in the preparation of example 1 but starting from intermediate 51 gave the title compound as white crystals after recrystallization from MeOH/MeCN/DMF in a 35% yield.

MP: 238-248° C.

Analysis for C₃₀H₃₂ClN₃O₄

Calculated: C, 67.47; H, 6.04; N, 7.87. Found: C, 67.08; H, 6.31; N, 7.81.

### EXAMPLE 9

4-(4-chloro-benzoylamino)-N-[4-(4-naphthalen-2-yl-piperidin-1-yl]-butyl}-benzamide

The same method was employed as in the preparation of example 1 but starting from intermediate 56 gave the title compound as white crystals after recrystallization from DMF/EtOH in a 65% yield.

MP: 270° C.

Analysis for C₃₃H₃₄ClN₃O₂ (3H₂O)

Calculated: C, 66.71; H, 6.79; N. 7.07. Found: C, 66.65; H, 6.45; N, 7.18

### EXAMPLE 10

4-(4-chloro-benzoylamino)-N-{4-[4-(5,6,7,8-tetrahydro-naphthalen-2-yl)-piperidin-1-yl]-butyl}-benzamide

The same method was employed as the preparation of example 1 but starting from intermediate 59 gave the title compound as crystals after recrystallization from EtOH in a 27% yield.

MP: 285° C.

Analysis for C₃₃H₃₈ClN₃O₂ (2H₂O)

Calculated: C, 68.32; H, 7.3; N, 7.24. Found: C, 68.02; H, 6.57; N, 7.31

### EXAMPLE 11

4-(4-chloro-benzoylamino)-N-[4-(4-naphthalen-1-yl-piperidin-1-yl]-butyl}-benzamide

The same method was employed as in the preparation of example 1 but starting from intermediate 64 gave the title compound as white crystals after recrystallization from DMF/EtOH in a 57% yield.

MP: 264° C.

Analysis for C₃₃H₃₄ClN₃O₂ (3H₂O)

Calculated: C, 66.71; H, 6.79; N, 7.07. Found: C, 66.83; H, 6.34; N, 7.2

### EXAMPLE 12

4-(4-chloro-benzoylamino)-N-{4-14-(2-trifluoroethoxy-4-methyl-phenyl)-piperidin-1-yl]-butyl}-benzamide

To a solution of the intermediate 13 (1.03 g, 3 mmol) in dry THF (100 mL), MeOH (250 mL) and DCM (100 mL) was added the intermediate 66 (0.82 g, 3 mmol.). The reaction was stirred at rt for 30 min and AcOH (1 mL) was added.

Then sodium triacetoxyborohydride (1.0 g, 2 eq.) was added and the reaction was stirred for 24 hours at rt. After evaporation under reduced pressure, the residue was taken up in DCM (350 mL) and washed with brine (75 mL). The organic phase was separated, dried over Na₂SO₄ and evaporated off. The residue was flash chromatographed using MeOH/DCM (5/95) to give the title as a white powder. Recrystallization from CH₃CN gave the title compound (1.1 g, 1.8 mmol).

MP: 226° C.

Analysis for C₃₂H₃₅ClF₃N₃O₃

Calculated: C, 63.84; H, 5.86; N, 6.98. Found: C, 63.56; H, 5.6; N, 6.89

### EXAMPLE 13

4′-Trifluoromethyl-biphenyl-4-carboxylic acid {4[4-(2-methylsulfanyl-phenyl)-piperidin-1-yl]-butyl}-amide

The same method was employed as in the preparation of example 7 but starting from intermediate 70 gave the title compound as white crystals after recrystallization from CH₃CN in a 56% yield.

MP: 191-192° C.

LC/MS (APCI): [M+H⁺] 527 C₃₀H₃₃F₃N₂OS

### EXAMPLE 14

4′-Trifluoromethyl-biphenyl-4-carboxylic acid {4-[4-(1−methyl-1H-indol-3-yl)-piperidin-1-yl]-butyl}-amide

A solution of intermediate 74 (0.4 g, 1.4 mmol) in DMF was treated with the intermediate 38 (0.34 g, 0.9 eq.), EDCI (0.53 g, 2.0 eq.), HOBt (0.37 g, 2.0 eq.) and TEA (0.38 mL, 2.0 eq.). The resulting mixture was stirred for 16 hours at rt. H₂O was added to the reaction and the precipitate formed was filtered off, washed with water and dried. Recrystallization from CH₃CN gave the title compound as white crystals in a 38% yield.

MP: 205-206° C.

LC/MS (APCI): [M+H+] 534 C₃₂H₃₄F₃N₃O

### EXAMPLE 15

4′-Trifluoromethyl-biphenyl-4-carboxylic acid {4-[4-(1H-indol-3-yl)-piperidin-1-yl]butyl}-amide

The same method was-employed as in the preparation of example 14 but starting from intermediate 78 to give the title compound as white crystals in a 66% yield after recrystallization from CH₃CN

MP: 194195° C.

LC/MS (APCI): [M+H+] 520 C₃₁H₃₂F₃N₃O

### EXAMPLE 16

4′-Trifluoromethyl-biphenyl-4-carboxylic acid [4-(4-benzo[b]thiophen-3-yl-piperidin-1-yl)-butyl]-amide

The same method was employed as in the preparation of example 14 but starting from intermediate 85 to give the title compound as white solid in a 87% yield after recrystallization from CH₃CN.

MP: 264° C.

LC/MS (APCI): (M+H⁺] 537 C₃₁H₃₁F₃N₂OS

### EXAMPLE 17

4-(4-chloro-benzoylamino)-N-{4-[4-(2,4-dimethoxy-phenyl)-piperazin-1-yl]-butyl}-benzamide hydrochloride

The same method was employed as in the preparation of example 1 but starting from the intermediate 87 gave the title compound as white crystals after recrystallization from MeOH/DCM (90/10/) in a 69.5% yield. The chlorhydrate was formed in addition of a 1N HCl solution in hot MeOH/DCM.

MP: 261° C.

Analysis for C₃₀H₃₅ClN₄O₄₀.2HCl

Calculated: C, 57.08; H, 5.94; N, 8.87. Found: C, 56.84; H, 5.98; N, 9.02

### EXAMPLE 18

4-(4-Chloro-benzoylamino)-N-{4-[4-(2,4-dimethoxy-phenyl)-[1,4]diazocan-1-yl]-butyl}-benzamide

The same method was employed as in the preparation of example 1 but starting from intermediate 90 to give the title compound as yellow crystals after recrystallization from EtOAc in 16% yield.

MP: 229° C.

Analysis for C₃₁H₃₇ClN₄O₄, (0.5H₂O):

Calculated: C, 64.85; H, 6.67; N, 9.76. Found: C, 64.94; H, 6.77; N, 9.74.

### EXAMPLE 19

4′-Trifluoromethyl-biphenyl-4-carboxylic acid {4-[4-(2-ethoxy-4-methyl-phenylamino)-piperidin-1-yl]-butyl}-amide

To a solution of intermediate 96 (0.7 g, 2.3 mmol) in dry DCM (20 mL) was added the intermediate 38 (0.62 g, 0.95 eq.), EDCI (0.53 g, 1.2 eq.), HOBt (0.37 g, 1.2 eq.) and TEA (0.7 mL, 2.0 eq.). The resulting mixture was stirred for 16 hours at rt. The residue was washed with a 1N NaOH solution and brine. The organic layer was dried over Na₂SO₄ and evaporated off. Recrystallization from CH₃CN gave the title compound as white crystals in a 63% yield.

MP: 162° C.

LC/MS (APCI): [M+H+] 554 C₃₂H₃₈F₃N₃O₂

### EXAMPLE 20

4′-Trifluoromethyl-biphenyl-4-carboxylic Acid (4-{4-[benzenesulfonyl-(2-ethoxy-4-methyl-phenyl)-amino]-piperidin-1-yl}butyl)-amide

To a solution of example 19 (0.7 g, 1.4 mmol) in DCM (20 mL) was added TEA (0.6 mL, 3.0 eq.) and phenyl sulfonyl chloride (0.65 mL, 3.5 eq.). The reaction was stirred to rt for 3 days and treated with water. The organic phase was washed with a 1N NaOH solution, water and brine, dried over Na₂SO₄ and evaporated off. Purification by flash chromatography using DCM/MeOH, 90/10 as eluent gave the title compound (0.23 g, 0.33 mmol) which was crystallized from Et₂O in a 23% yield.

MP: 110° C.

LC/MS (APCI): [M+H+] 694 C₃₈H₄₂F₃N₃O₄S

### EXAMPLE 21

4′-Trifluoromethyl-biphenyl-4-carboxylic acid {4-[4-(naphtalen-1-yloxy)-piperidin-1-yl]-butyl}-amide

The same method was employed as in the preparation of example 14 but starting from intermediate 100 to give the title compound as white crystals in a 62% yield after recrystallization from CH₃CN.

MP: 166° C.

LC/MS (APCI): [M+H⁺] 547 C₃₃H₃₃F₃N₂O₂

### EXAMPLE 22

4-(4-chloro-benzoylamino)-N-{4-[4-(2-methoxy-4-methyl-phenyl)-piperazin-1-yl]-butyl}-benzamide Hydrochloride

The same method was employed as in the preparation of example 2 but starting from intermediate 103 gave the title compound as white crystals after precipitation from DCM/MeOH (90/10) in a 95% yield. The chlorhydrate was formed in addition of a 1N HCl solution in hot DMF.

MP: 227° C.

Analysis for C₃₀H₃₅ClN₄O₃ 3HCl:

Calculated: C, 55.91; H, 5.94; N, 8.69. Found: C, 56.28; H. 5.76; N, 8.55

### EXAMPLE 23

4′-Trifluoromethyl-biphenyl-4-sulfonic acid {4-[4-(2-ethoxy-4-methyl-phenyl)-piperidin-1-yl]-butyl}-amide Hydrochloride

A solution of intermediate 105 (0.136 g, 0.47 mmol), TEA (70 μL, 1.0 eq.) and the available 4′-Trifluoromethyl-biphenyl-4-sulfonic acid (0.15 g, 1 eq.) in THF (10 mL) was stirred for 2 hours at rt. The solution was evaporated off, treated with water and extracted with DCM. The organic phase was dried over Na₂SO₄, filtrated, evaporated off, to give after purification by flash chromatography using DCM/MeOH 95/5 as eluent the title compound as a white powder. The chlorhydrate was obtained from a HCl/Et₂O solution (0.12 g, 0.2 mmol) in a 42% yield.

MP: 188-190° C.

LC/MS (APCI): [M+H⁺] 575 C₃₁H₃₇F₃N₂O₃S

### EXAMPLE 24

5-[4-(2-Ethoxy-4-methyl-phenyl)-piperidin-1-yl]-pentanoic acid (4′-trifluoromethyl-biphenyl-4-yl)-amide

A solution of intermediate 108 (2.0 g, 6.26 mmol), HATU (4.1 g, 19.7 mmol) TEA (5 mL) and intermediate 107 (1.27 g, 0.55 eq.) in THF (100 ml) was stirred at rt for 18 hours. The mixture was concentrated and the residue was dissolved in DCM and washed with water and a saturated NaHCO₃ solution. The solvent was evaporated off and the residue was purified by flash chromatography using DCM/MeOH (9/1) as eluent. The powder was recrystallized from CH₃CN to give the title compound as white crystals (0.2 g, 0.37 mmol) in a 7% yield.

MP: 178° C.

LC/MS (APCI): [M+H⁺] C₃₂H₃₇F₃N₂O₂ 539

### EXAMPLE 25

4-{5-[4-(1-Methoxy-naphtalen-2-yl)-piperidin-1-yl]-pentyloxy}-biphenyl-4-carbonitrile

To a solution of intermediate 109 (0.72 g, 2.1 mmol) in acetone (20 mL) was added potassium carbonate (0.58 g, 2.0 eq.) and the intermediate 113 (0.5 g, 1.0 eq.). The reaction was stirred to reflux for 24 hours. After cooling, the reaction was filtered off and the solvent was evaporated in vacuo. After purification by flash chromatography, using DCM/MeOH (90/10) as eluent, and recrystallization from MeOH, the title compound was obtained as a white crystals in a 15% yield.

MP: 153-154° C.

¹H NMR (CDCl₃, 300 MHz) δ 8.0 (d, 1H), 7.8 (d, 1H), 7.8-7.2 (m, 10H), 4.0 (t, 2H), 3.8 (s, 3H), 3.1 (m, 3H), 2.4 (m, 2H), 2.1 (m, 2H), 1.8 (m, 5H), 1.4 (m, 5H).

### EXAMPLE 26

4′-{4-[4-(1-Methoxy-naphtalen-2-yl)-piperidin-1-yl]-butoxy}-biphenyl-4-carbonitrile

The same method was employed as in the preparation of example 25 but starting from intermediate 114 gave the title compound as white crystals in a 13% yield after recrystallization from MeOH.

MP: 142° C.

LC/MS (APCI): [M+H⁺] 491 C₃₃H₃₄N₂O₂

### EXAMPLE 27

4-Methyl-2-(4-trifluoromethyl-phenyl)-thiazole-5-carboxylic acid {4-[4-(4-isopropyl-2-methoxy-phenyl)-piperidin-1-yl]-butyl}-amide

The same method was employed as in the preparation of example 1 but starting from intermediate 33 and the available 4-Methyl-2-(4-trifluromethyl-phenyl)-thiazole-5-carboxylic acid gave the title compound as white crystals after recrystallization from MeCN in a 54% yield.

MP: 170° C.

Analysis for C₃₁H₃₈F₃N₃O₂S, (0.4H₂O):

Calculated: C, 71.75; H, 6.57; N, 6.78. Found: C, 71.53; H, 6.22; N, 6.88

### EXAMPLE 28

2-(4-Chloro-phenyl)-1-methyl-1H-indole-5-carboxylic Acid {4-[4-(2-ethoxy-4-methyl-phenyl)-piperidin-1-yl]-butyl}-amide

A solution of the intermediate 105 (0.29 g, 1 mmol.) in DMF was treated with intermediate 119 (0.286 g, 1 eq.), HATU (0.423 g, 1.1 eq.) and TEA (420 μL, 3 eq.). The resulting mixture was stirred for 18 hours at rt. The solvent was evaporated off. The residue was taken up in DCM and washed with a 1N NaOH solution and brine. The organic layer was dried over Na₂SO₄ and evaporated off. The title compound (0.27 g, 0.5 mmol.) was obtained after recrystallization from CH₃CN as a yellow solid in a 48% yield.

MP: 174-175° C.

LC/MS (APCI): [M+H+] 559 C₃₄H₄₀ClN₃O₂

### EXAMPLE 29

2-(4-Trifluoromethyl-phenyl)-benzofuran-5-carboxylic Acid {4-[4-(2-ethoxy-4-methyl-phenyl)-piperidin-1-yl]-butyl}-amide

The same method was employed as in the preparation of example 28 but starting from intermediate 121 gave the title compound as white needles after recrystallization from CH₃CN in a 53% yield.

MP: 200° C.

LC/MS (APCI): [M+H+] 579 C₃₄H₃₇F₃N₂O₃

### EXAMPLE 30

2-(4-Chloro-phenyl)-benzofuran-5-carboxylic acid {4-[4-(2-ethoxy-4-methylphenyl)-piperidin-1-yl]-butyl}-amide

The same method was employed as in the preparation of example 28 but starting from intermediate 124 gave the title compound as white needles after recrystallization from CH₃CN in a 45% yield.

MP: 145-158° C.

LC/MS (APCI): [M+H+] 546 C₃₃H₃₇ClN₂O₃

### EXAMPLE 31

2-(3,4-Dichloro-phenyl)-benzofuran-5-carboxylic acid {4-[4-(1-cyclopropylmethoxy-5,6,7,8-tetrahydro-naphtalen-2-yl)-piperidin-1-yl]-butyl}-amide

The same method was employed as in the preparation of example 1 but starting from intermediate 131 and intermediate 125 gave the title compound as white solid after flash chromatography using DCM/MeOH (90/1-0), as eluent in a 45% yield.

MP: 175-176° C.

LC/MS (APCI): [M+H+] 646 C₃₈H₄₂Cl₂N₂O₃

### EXAMPLE 32

2-(6-Trifluoromethyl-pyridin-3-yl)-benzofuran-5-carboxylic acid {4-[4-(1-cyclopropylmethoxy-5,6,7,8-tetrahydronaphtalen-2-yl)-piperidin-1-yl]-butyl}-amide

The same method was employed as in the preparation of example 31 but starting from intermediate 133 to give the title compound as beige crystals in a 62% yield after recrystallization from CH₃CN

MP: 186

LC/MS (APCI): [M+H+] 646 C₃₈H₄₂F₃N₃O₃

### EXAMPLE 33

N-{4-[4-(2-Ethoxy-4-methyl-phenyl)-piperidin-1-yl]-butyl}-4-[2-(4-trifluoromethylphenyl)-vinyl]-benzamide

To a solution of intermediate 136 (0.584 g, 2 mmol) in THF (100 mL) was added HOBT (0.540 g, 2.0 eq.), intermediate 105 (0.522 g, 1.8 mmol), EDCI (0.767 g, 2.0 eq.) and TEA (10 mL).

The resulting mixture was stirred 18 hours at rt and then filtered. The solution was evaporated off and treated with water (100 mL) to give an orange precipitate. The product was filtered off and purified by flash chromatography using DCM/MeOH 95/5 as eluent to give the title compound as a white powder (0.3 g, 0.5 mmol) in a 26% yield

MP: 210° C.

LC/MS (APCI): [M+H⁺] 565 C₃₄H₃₉F₃N₂O₂

### EXAMPLE 34

N-{4-[4-(2-Ethoxy-4-methyl-phenyl)-piperidin-1-yl]-butyl}4-(4-trifluoromethyl-benzyloxy)-benzamide

The same method was employed as in the preparation of example 33 but starting from intermediate 138 gave the title compound as white needles after recrystallization from CH₃CN in a 62% yield.

MP: 180-182° C.

LC/MS (APCI): [M+H⁺] 569 C₃₃H₃₉F₃N₂O₃

### EXAMPLE 35

4-[2-(3,5-dichlorophenyl)ethenyl]-N-{4-[4-(2,4-dimethoxyphenyl)piperazin-1-yl]-butyl}-benzamide

To a solution of intermediate 87 (909 mg, 3.1 mmol) in DMF (35 mL) was added HOBT (461 mg, 3.4 mmol), EDCI (654 mg, 3.4 mmol), TEA (0.65 mL, 1.5 eq.) and intermediate 140 (1 g, 3.4 mmol). The reaction heated to 60° C. and followed by TLC (DCM/MeOH: 9:1; Rf=0.55). When all of the starting material had disappeared, the reaction cooled to rt and the solvent removed under vaccum. The residue treated with 1N NaOH and the product extracted 5×100 ml EtOAc. The organic layers combined, washed with 1N HCl, dried over Na₂SO₄ and the solvent removed in vacuo. The desired product precipated with DCM to afford 1.34 g (76%) as a white solid.

¹H NMR (CDCl₃, 250 MHz) δ 8.85 (t, 1H), 8.05 (d, 2H), 7.8 (d, 2H), 7.25 (d, 1H), 6.75 (d, 1H), 6.65 (dd, 1H), 3.9 (s, 3H), 3.85 (s, 3H), 3.8 (m, 2H), 3.6-3.4 (m, 10H), 2.0-1.65 (m, 4H).

### EXAMPLE 36

4-[2-(3,5-dichloro-phenyl)-ethyl]-N-{4-[4-(2,4-dimethoxy-phenyl)-piperazin-1-yl]-butyl∵-benzamide

To a solution of example 35 (500 mg, 0.88 mmol) in THF/MeOH (20 mL/5 mL) was added Pd/C (spatula tip) and the reaction degassed 3×N₂ followed by 3×H₂. The reaction was then stirred under H₂ (1 atm) while followed by TLC (DCM/MeOH: 8:2; Rf=0.7). When all of the starting material had disappeared, the reaction filtered through celite and the solvent removed under vaccum. The residue flash chromatographed (silica gel; DCM/MeOH: 9/1) to give 380 mg (76%) desired product as a white solid.

MP: 126-128° C.

Analysis for C₃₁H₃₇Cl₂N₃O₃, (0.1 C₄H₁₀O):

Calculated: C 65.25; H 6.63; N 7.27; Found: C 65.69; H 7.05; N 7.38.

### EXAMPLE 37

4-(4-Benzoyl)-N-4-[4-(4-isopropyl-2-methoxy-phenyl)-piperidin-1-yl]-butyl}-benzamide hydrochloride

A solution of intermediate 33 (0.2 g, 0.66 mmol) in DMF (5 mL) was treated with 4-Benzoylbenzoic acid (0.15 g, 1.0 eq.), EDCI (1.5 eq.), HOBt (1.5 eq.) and TEA (1.5 eq.). The resulting mixture was stirred for 16 hours at rt. The solvent was evaporated off. The residue was taken up in DCM and washed with a 1N NaOH solution and brine. The organic layer was dried over Na₂SO₄ and evaporated off. The residue was dissolved in a minimum amount of hot DMF and treated with a 1N HCl solution to give the title compound as a white solid in a 34% yield.

MP: 138° C.

Analysis for C₃₃H₄₀N₂O₃ (2 HCl)

Calculated: C, 67.68; H, 7.23; N, 4.78. Found: C, 67.59; H, 7.68; N, 4.94

### EXAMPLE 38

4′-trifluoromethyl-biphenyl-4-carboxylic acid {4-[4-(2,4-diethoxy-benzyl)-piperidin-1-yl]-butyl}amide

A solution of intermediate 145 (0.210 g, 0.63 mmol, 1.05 eq) in dry DMF was treated with intermediate 38 (0.16 g, 0.6 mmol), HATU (0.23 g, 0.6 mmol, 1 eq) and TEA (0.255 ml, 1.8 mmol, 3 eq). The resulting mixture was stirred for 48 hours at rt. The solvent was evaporated off. The residue was taken up in water, a 1N NaOH solution (5 ml) was added and the mixture was extracted with DCM, washed with brine, dried over Na₂SO₄, and then concentrated in vacuo. The residue was purified by flash chromatography using DCM/MeOH (95/5) to give the title compound as a white powder (0.100 g, 0.17 mmol) in a 29% yield. MP: 136-137° C.

LC/MS: [M+H+] 583 C₃₄H₄₂F₃N₂O₃

### EXAMPLE 39

4-(4-chloro-benzoylamino)-N-4-[4-(2,4-dimethoxy-benzoyl)-piperidin-1-yl]-buty}-benzamide

The same method was employed as in the preparation of example 2 but starting from intermediate 147 gave the title compound as a as a white solid (0.7 g, 1.2 mmol) in a 40% yield after purification by column chromatography using DCM/MeOH 90/10 as eluent and after crystallisation in EtOH.

MP: 209-210° C.

LC/MS: [M+H+] 578 C₃₂H₃₆ClN₃O₅

### EXAMPLE 40

4′-Cyano-biphenyl-4-carboxylic acid {4-[4-(1-methyl-1H-indol-3-yl)-piperidin-1-yl]-butyl}-amide

The same method was employed as in the preparation of example 14 but starting from intermediate 74 and the available 4′-Cyano-biphenyl-4-carboxylic acid gave the title compound as white solid after recrystallization from MeCN in a 33% yield.

MP: 180° C.

LC/MS (APCI): [M+H+] 491 C₃₂H₃₄N₄O

### EXAMPLE 41

4-(4-chloro-benzoylamino)-N-{4-[4-(5-methyl-2-piperidin-4-yl-phenol)]-butyl}-benzamide Hydrochloride

To a solution of intermediate 149 (3.0 g, 15.7 mmol) in dry THF (70 mL) and MeOH (200 mL) was added the intermediate 13 (5.4 g, 1.0 eq.). The reaction was stirred at rt for 30 min and AcOH (1.5 eq) was added. Then sodium triacetoxyborohydride (1.2 eq.) was added and the reaction was stirred for 24 hours at 80° C. After cooling, the solvent was evaporated and H₂O was added. The precipitate was filtered off, treated with al N HCl solution and dried to give the title compound as a white powder in 76% yield.

MP: 254° C.

Analysis for C₃₀H₃₄ClN₃O₃ (1.4 HCl)

Calculated: C, 63.09; H, 6.25; N, 7.36. Found: C, 63.26; H, 6.49; N, 7.47

### EXAMPLE 42

4-(4-chloro-benzoylamino)-N-{4-[4-(5-ethyl-2-piperidin-4-yl-phenol)]-butyl}-benzamide Acetate

The same method was employed as in the preparation of example 41 but starting from intermediate 151 gave the title compound as a white solid after recrystallization from MeOH in 64% yield.

MP: 213° C.

Analysis for C₃₁H₃₆ClN₃O₃(1 CH₃CO₂H)

Calculated: C, 69.78; H, 7.41; N, 7.18. Found: C, 69.91; H, 7.45; N, 7.16

### EXAMPLE 43

4-(4-Chloro-benzoylamino)-N-{4-[4-(1-hydroxy-5,6,7,8-tetrahydro-naphtalen-2-yl)-piperidin-1-yl]-butyl}-benzamide Hydrochloride

The same method was employed as in the preparation of example 1 but starting from intermediate 2 and 154 to give the title compound as white crystals after formation of chlorhydrate from a hot HCl 1N/EtOH solution in a 52% yield.

MP: 268° C.

LC/MS(ES): M+ 559 C₃₃H₃₈ClN₃O₃

### EXAMPLE 44

4-(4-Chloro-benzoylamino)-N-4-[4-(1−hydroxy-naphtalen-2-yl)-piperidin-1-yl]-butyl}-benzamide Hydrochloride

The same method was employed as in the preparation of example 1 but starting from the intermediates 2 and 157 to give the title compound as a white powder in a 58% yield.

MP: 274° C.

LC/MS(APCI): [M+H⁺] 550 C₃₃H₃₄N₃O₃Cl

Biological Assays

In Vitro Assay:

HepG₂ cells, stably transfected with a construct comprising the the LDL-r promoter and the luciferase reporter gene, were seeded at 50.000 cells/well in 96 well plates. After 1 day, cells were incubated with compounds for 24 hours in RPMI medium containing 2% of lipoprotein-deficient serum. Compounds were tested from 10⁻⁶M to 10⁻⁹M. Cell lysates were prepared and the luciferase activity was measured by the luciferase assay system (Promega). Induction of luciferase activity was calculated taking untreated cells as control and ED₅₀ of each compounds was determinated compared to the ED₅₀ of an internal standart.

In Vivo Assay:

Compounds were prepared for oral administration by milling with 0.5% hydroxypropylmethylcellulose and 5% Tween 80. Hamsters were fed for 2 weeks with a diet containing 0.2% of cholesterol and 10% of coconut oil. Then compounds were administrated once a day for 3 days, from 20 to 0.2 mg/kg. Plasma lipid levels including total cholesterol, VLDULDL cholesterol, VLDL/LDL triglycerides and HDL-cholesterol were determinated after ultracentrifugation (density 1.063 g/ml to separate VLDL/LDL fraction and HDL fraction) using the Biomerieux enzymatic kit. Reductions in VLDL/LDL cholesterol and TG plasmatic levels were calculated taking solvant treated animals as control and ED₅₀ of each compound was determined.

| Example | In vitro (IC₅₀) (nm) |
|---|---|
| 7 | 4 |
| 40 | 10 |
| 20 | 117 |
| 33 | 30 |
| 31 | 20 |
| 26 | 1 |
| 32 | 13 |

Tablet Compositions

The following compositions A and B can be prepared by wet granulation of ingredients (a) to (c) and (a) to (d) with a solution of povidone, followed by addition of the magnesium stearate and compression.

Composition A

| | | mg/tablet | mg/tablet |
|---|---|---|---|
| (a) | Active ingredient | 250 | 250 |
| (b) | Lactose B.P. | 210 | 26 |
| (c) | Sodium Starch Glycollate | 20 | 12 |
| (d) | Povidone B.P. | 15 | 9 |
| (e) | Magnesium Stearate | 5 | 3 |
| | | 500 | 300 |

Composition B

| | | mg/tablet | mg/tablet |
|---|---|---|---|
| (a) | Active ingredient | 250 | 250 |
| (b) | Lactose 150 | 150 | — |
| (c) | Avicel PH 101 | 60 | 26 |
| (d) | Sodium Starch Glycollate | 20 | 12 |
| (e) | Povidone B.P. | 15 | 9 |
| (f) | Magnesium Stearate | 5 | 3 |
| | | 500 | 300 |

Composition C

| | mg/tablet |
|---|---|
| Active ingredient | 100 |
| Lactose | 200 |
| Starch | 50 |
| Povidone | 5 |
| Magnesium Stearate | 4 |
| | 359 |

The following compositions D and E can be prepared by direct compression of the admixed ingredients. The lactose used in composition E is of the direct compression type.

Composition D

| | mg/tablet |
|---|---|
| Active ingredient | 250 |
| Magnesium Stearate | 4 |
| Pregelatinised Starch NF15 | 146 |
| | 400 |

Composition E

| | mg/tablet |
|---|---|
| Active ingredient | 250 |
| Magnesium Stearate | 5 |
| Lactose | 145 |
| Avicel | 100 |
| | 500 |

Composition F (Controlled release composition)

| | | mg/tablet |
|---|---|---|
| (a) | Active ingredient | 500 |
| (b) | Hydroxypropylmethylcellulose | 112 |
| | (Methocel K4M Premium) | |
| (c) | Lactose B.P. | 53 |
| (d) | Povidone B.P.C. | 28 |
| (e) | Magnesium Stearate | 7 |
| | | 700 |

The composition can be prepared by wet granulation of ingredients (a) to (c) with a solution of povidone, followed by addition of the magnesium stearate and compression.

Composition G (Enteric-Coated Tablet)

Enteric-coated tablets of Composition C can be prepared by coating the tablets with 25 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

Composition H (Enteric-Coated Controlled Release Tablet)

Enteric-coated tablets of Composition F can be prepared by coating the tablets with 50 mg/tablet of an enteric polymer such as cellulose acetate phthalate, polyvinylacetate phthalate, hydroxypropylmethyl-cellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(ii) Capsule Compositions

Composition A

Capsules can be prepared by admixing the ingredients of Composition D above and filling two-part hard, gelatin capsules with the resulting mixture. Composition B (infra) may be prepared in a similar manner.

Composition B

| | | mg/capsule |
|---|---|---|
| (a) | Active ingredient | 250 |
| (b) | Lactose B.P. | 143 |
| (c) | Sodium Starch Glycollate | 25 |
| (d) | Magnesium Stearate | 2 |
| | | 420 |

Composition C

| | mg/capsule |
|---|---|
| (a) Active ingredient | 250 |
| (b) Macrogol 4000 BP | 350 |
| | 600 |

Capsules can be prepared by melting the Macrogol 4000 BP, dispersing the active ingredient in the melt and filling two-part hard gelatin capsules therewith.

Composition D

| | mg/capsule |
|---|---|
| Active ingredient | 250 |
| Lecithin | 100 |
| Arachis Oil | 100 |
| | 450 |

Capsules can be prepared by dispersing the active ingredient in the lecithin and arachis oil and filling soft, elastic gelatin capsules with the dispersion.

Composition E (Controlled release capsule)

| | | mg/capsule |
|---|---|---|
| (a) | Active ingredient | 250 |
| (b) | Microcrystalline Cellulose | 125 |
| (c) | Lactose BP | 125 |
| (d) | Ethyl Cellulose | 13 |
| | | 513 |

The controlled release capsule composition can be prepared by extruding mixed ingredients (a) to (c) using an extruder, then spheronising and drying the extrudate. The dried pellets are coated with a release controlling membrane (d) and filled into two-part, hard gelatin capsules.

Composition F (Enteric capsule)

| | | mg/capsule |
|---|---|---|
| (a) | Active ingredient | 250 |
| (b) | Microcrystalline Cellulose | 125 |
| (c) | Lactose BP | 125 |
| (d) | Cellulose Acetate Phthalate | 50 |
| (e) | Diethyl Phthalate | 5 |
| | | 555 |

The enteric capsule composition can be prepared by extruding mixed ingredients (a) to (c) using an extruder, then spheronising and drying the extrudate. The dried pellets are coated with an enteric membrane (d) containing a plasticizer (e) and filled into'two-part, hard gelatin capsules.

Composition G (Enteric-Coated Controlled Release Capsule)

Enteric capsules of Composition E can be prepared by coating the controlled-release pellets with 50 mg/capsule of an enteric polymer such as cellulose acetate phthalate, polyvinylacetate phthalate, hydroxypropylmethylcellulose phthalate, or anionic polymers of methacrylic acid and methacrylic acid methyl ester (Eudragit L). Except for Eudragit L, these polymers should also include 10% (by weight of the quantity of polymer used) of a plasticizer to prevent membrane cracking during application or on storage. Suitable plasticizers include diethyl phthalate, tributyl citrate and triacetin.

(iii) Intravenous injection composition

| | |
|---|---|
| Active ingredient | 0.200 g |
| Sterile, pyrogen-free phosphate buffer (pH 9.0) to | 10 ml |

The active ingredient is dissolved in most of the phosphate buffer at 35-40° C., then made up to volume and filtered through a sterile micropore filter into sterile 10 ml glass vials (Type 1) which are sealed with sterile closures and overseals.

(iv) Intramuscular injection composition

| | |
|---|---|
| Active ingredient | 0.20 g |
| Benzyl Alcohol | 0.10 g |
| Glycofurol 75 | 1.45 g |
| Water for Injection q.s. to | 3.00 ml |

The active ingredient is dissolved in the glycofurol. The benzyl alcohol is then added and dissolved, and water added to 3 ml. The mixture is then filtered through a sterile micropore filter and sealed in sterile 3 ml glass vials (Type 1).

(v) Syrup composition

| | | |
|---|---|---|
| Active ingredient | 0.25 | g |
| Sorbitol Solution | 1.50 | g |
| Glycerol | 1.00 | g |
| Sodium Benzoate | 0.005 | g |
| Flavour | 0.0125 | ml |
| Purified Water q.s. to | 5.0 | ml |

The sodium benzoate is dissolved in a portion of the purified water and the sorbitol solution added. The active ingredient is added and dissolved. The resulting solution is mixed with the glycerol and then made up to the required volume with the purified water.

(vi) Suppository composition

| | mg/suppository |
|---|---|
| Active ingredient | 250 |
| Hard Fat, BP (Witepsol H15 - Dynamit NoBel) | 1770 |
| | 2020 |

One-fifth of the Witepsol H15 is melted in a steam-jacketed pan at 45° C. maximum. The active ingredient is sifted through a 200 lm sieve and added to the molten base with mixing, using a Silverson fitted with a cutting head, until a smooth dispersion is achieved. Maintaining the mixture at 45° C., the remaining Witepsol H15 is added to the suspension which is stirred to ensure a homogenous mix. The entire suspension is then passed through a 250 lm stainless steel screen and, with continuous stirring, allowed to cool to 40° C. At a temperature of 38-40° C., 2.02 g aliquots of the mixture are filled into suitable plastic moulds and the suppositories allowed to cool to room temperature.

(vii) Pessary composition

| | mg/pessary |
|---|---|
| Active ingredient (63 lm) | 250 |
| Anhydrous Dextrose | 380 |
| Potato Starch | 363 |
| Magnesium Stearate | 7 |
| | 1000 |

The above ingredients are mixed directly and pessaries prepared by compression of the resulting mixture.

(viii) Transdermal composition

| | |
|---|---|
| Active ingredient | 200 mg |
| Alcohol U.S. Pat. No. | 0.1 ml |
| Hydroxyethyl cellulose | |

The active ingredient and alcohol USP are gelled with hydroxyethyl cellulose and packed in a transdermal device with a surface area of 10 cm².

1. Use of a compound of formula (I) 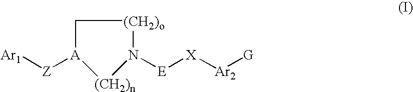
wherein
Ar₁ represents
(iii) phenyl, naphthyl, or phenyl fused by a C₃₋₈cycloalkyl,
(iv) heterocyclyl selected from the group consisting of monocyclic radicals and fused polycyclic radicals, wherein said radicals contain a total of from 5-14 ring atoms, wherein said radicals contain a total of from 14 ring heteroatoms independently selected from oxygen, nitrogen and sulfur, and wherein individual rings of said radicals may be independently saturated, partially unsaturated, or aromatic, providing that at least one ring is aromatic,
where Ar₁ optionally optionally bears 1-4 groups independently represented by R¹;
R¹ is selected from halogen, —S(C₁₋₄ alkyl), —O—(C₀₋₄ alkylene)-R² or C₀₋₄alkylene)-R², where each alkylene group may additionally incorporate an oxygen in the chain, with the proviso that there are at least two carbon atoms between any chain heteroatoms;
R² represents
(v) hydrogen, C₁₋₄ perfluoroalkyl, C₁₋₄perfuoroalkoxy,
(vi) phenyl, phenyl fused by a C₃₋₈cycloalkyl, naphthyl or a 5- or 6-membered heteroaromatic group, optionally substituted by one or two groups independently selected from halogen, C₁₋₄ alkyl, hydroxy, C₁₋₄ alkoxy, amino, C₁₋₄ alkylamino and di-C₁₋₄ alkylamino,
(vii) C₃₋₈cycloalkyl or a monocyclic heterocyclyl radical containing a total of 3-7 ring atoms, wherein said radical contains a total of from 14 ring heteroatoms independently selected from oxygen, nitrogen and sulfur, wherein said radical may be independently saturated, partially unsaturated, or aromatic, and where, the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-C₁₋₄ alkylamino, or
(viii) amino, C₁₋₄ alkylamino or di-C₁₋₄alkylamino,
with the proviso that there are at least two carbon atoms between any chain heteroatoms;
Z is a direct link, oxo, —O—, C(H)R³, —N(R⁵)—, —N(SO₂R⁶)— or —SO₂—;
R³ is hydrogen, C₁₋₄ alkyl or phenyl, said phenyl optionally bearing one or two groups independently selected from halogen, C₁₋₄ alkyl, C₁₋₄ alkoxy and OH;
A is C—R⁴ or N;
n is an integer selected from 1-3;
o is an integer selected from 1-2;
R⁴ is hydrogen, C₁₋₄ alkyl, hydroxy or phenyl, said phenyl optionally bearing one or two groups indpendently selected from halogen, C₁₋₄ alkyl, C₁₋₄ alkoxy or hydroxy, or R³ forms a double bond between A and an adjacent ring carbon;
R⁵ is C₁₋₄ alkyl or phenyl;
R⁶ is C₁₋₄ alkyl or phenyl;
E is a C₁₋₆ alkylene group, optionally containing one or two double bonds or one triple bond and optionally incorporating an O, S or N(H or C₁₋₄ alkyl) group in the chain;
X is a direct link, —O—, oxo, —CON(H or C₁₋₄ alkyl)-, —N(H or C₁₋₄ alkyl)CO—, —N(H or C₁₋₄ alkyl)SO₂ or —SO₂N(H or C₁₋₄ alkyl)-;
Ar₂ is phenyl, a 5-6 membered heteroaromatic group or a bicyclic heteroaromatic group, where each group is optionally substituted by one or two groups independently selected from C₁₋₄ alkyl, halogen, hydroxy, C₁₋₄ alkoxy, C₁₋₆ acyl, C₁₋₆ acyloxy, amino, C₁₋₄ alkylamino and di-C₁₋₄ alkylamino groups;
G is hydrogen or —Y—Ar₃;
Y is a direct link, oxo, —O—, —N(H or C₁₋₄ alkyl)CO—, —CON(H or C₁₋₄ alkyl)-, —N(H or C₁₋₄ alkyl)SO₂— or —SO₂N(H or C₁₋₄ alkyl)-, —C₁₋₂ alkylene-, —O—C₁₋₂ alkylene- or —C₂₋₃alkenylene-;
Ar₃ represents
(vii) phenyl, naphthyl, or phenyl fused by a C₃₋₈cycloalkyl,
(viii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
where Ar₃ optionally bears 14 groups independently selected from hydroxy, alkyl, C₁alkoxy, C₂₋₄ alkenyl, C₂₋₄ alkenyloxy, C₁₋₄ perfluoroalkoxy, C₁₋₄ acylamino or an electron-withdrawing group;
or a physiologically acceptable salt, solvate or derivative thereof, in the manufacture of a medicament for the treatment of diseases ameliorated by LDL-r upregulation.

2. Use according to claim 1 where Ar₁ represents an optionally substituted phenyl, naphthyl, 1,2,3,4-tetrahydronaphthyl or bicyclic heteroaromatic group, where optional substitution is effected by R¹.

3. Use according to claim 1 or 2 where substitution on Ar₁ is represented by methylenedioxy or one, two or three groups independently selected from C₁₋₄ alkylhydroxy, C₁₋₄ alkoxy, —O—C₀₋₄alkylene-R², where R² represents C₁₋₄ perfluoroalkyl, a 5-6 membered heteroaromatic group, e.g. pyridyl or a C₃₋₈cycloalkyl.

4. Use according to any one of claims 1 to 3 where A is —C(H)—.

5. Use according to any one of claims 1 to 4 where Z is a direct link, —NH—, NSO₂Ph- or —O—.

6. Use according to any one of claims 1 to 5 where Integers o and n are 1 and 2 respectively.

7. Use according to any one of claims 1 to 6 where E is an n-butylene group.

8. Use according to any one of claims 1 to 7 where G is Y—Ar₃.

9. Use according to claim 8 where Y is an —N(H)CO— group or a direct link.

10. Use according to any one of claims 1 to 9 where Ar₂ is a bicyclic heteroaromatic group selected from benzofuranyl or indolyl, optionally substituted by C₁₋₄alkyl.

11. Use according to any one of claims 1 to 10 where Ar₃ is phenyl or a pyridyl group, substituted by a halogen, nitrile or C₁₋₄perfluoroalkyl.

12. Use of a compound of formula (Ia) 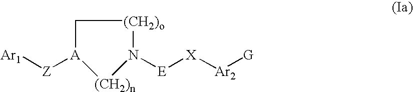
wherein
Ar₁ represents
(v) phenyl, naphthyl, or phenyl fused by a C₃₋₈cycloalkyl,
(vi) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
where Ar₁ optionally bears 1-4 groups independently represented by R¹;
R¹ is selected from halogen, —O—(C₀₋₄ alkylene)-R² or C₀₋₄alkylene)-R², where each alkylene group may additionally incorporate an oxygen in the chain, with the proviso that there are at least two carbon atoms between any chain heteroatoms;
R² represents
(i) hydrogen, C₁₋₄ perfluoroalkyl, C₁₋₄perfluoroalkoxy,
(ii) phenyl, phenyl fused by a C₃₋₈cycloalkyl, naphthyl or a 5- or 6-membered heteroaromatic group, optionally substituted by one or two groups independently selected from halogen, C₁₋₄ alkyl, hydroxy, C₁₋₄ alkoxy, amino, C₁₋₄ alkylamino and di-C₁₋₄ alkylamino,
(iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-C₁₋₄ alkylamino, or
(iv) amino, C₁₋₄ alkylamino or di-C₁₋₄alkylamino,
with the proviso that there are at least two carbon atoms between any chain heteroatoms;
Z is a direct link, oxo, —C(H)R³— or —SO₂—;
R³ is hydrogen, C₁₋₄ alkyl or phenyl, said phenyl optionally bearing one or two groups indpendently selected from halogen, C₁₋₄ alkyl, C₁₋₄ alkoxy and OH;
A is C—R⁴ or N;
n is an integer selected from 1-3;
o is an integer selected from 1-2;
R⁴ is hydrogen, C₁₋₄ alkyl, hydroxy or phenyl, said phenyl optionally bearing one or two groups indpendently selected from halogen, C₁₋₄ alkyl, C₁₋₄ alkoxy or hydroxy, or R⁴ forms a double bond between A and an adjacent ring carbon;
E is a C₁₋₄ alkylene group, optionally containing one or two double bonds or one triple bond;
X is a bond, —O—, oxo, —CON(H or C₁₋₄ alkyl)-, —N(H or C₁₋₄ alkyl)CO—, —N(H or C₁₋₄ alkyl)SO₂— or —SO₂N(H or C₁₋₄ alkyl)-;
Ar₂ is phenyl or a 5-6 membered heteroaromatic group, optionally substituted by one or two groups independently selected from C₁₋₄ alkyl, halogen, hydroxy, C₁₋₄ alkoxy, C₁₋₆ acyl, C₁₋₆ acyloxy, amino, C₁₋₄ alkylamino and di-C₁₋₄ alkylamino groups;
G is —Y—Ar₃;
Y is a bond, oxo, —O—, —N(H or C₁₋₄ alkyl)CO—, —CON(H or C₁₋₄ alkyl)-, —N(H or C₁₋₄ alkyl)SO₂— or —SO₂N(H or C₁₋₄ alkyl)-, C₁₋₂ alkylene or C₂₋₃alkenylene;
Ar₃ represents
(i) phenyl, naphthyl, or phenyl fused by a C₃₋₈cycloalkyl,
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
where Ar₃ optionally bears 1-4 groups independently selected from halogen, nitrile, C₁₋₄ alkyl, C₁₋₄ alkoxy, C₂₋₄ alkenyl, C₂₋₄ alkenyloxy, hydroxy, azido, C₁₋₄perfluoroalkyl, C₁₋₄perfluoroalkoxy, C₁₋₄ acyl, C₁₋₄ acyloxy, C₁₋₄ alkoxycarbonyl, aminocarbonyl, C₁₋₄ alkylaminocarbonyl; di-C₁₋₄ alkylaminocarbonyl, C₁₋₄ acylamino, amino, C₁₋₄ alkylamino or di-C₁₋₄ alkylamino groups;
or a physiologically acceptable salt, solvate or derivative thereof, in the manufacture of a medicament for the treatment of diseases ameliorated by LDL-r upregulation.

13. Use of a compound selected from:
4-(4-chloro-benzoylamino)-N-{4-[4-(2,4-dimethoxy-phenyl)-piperidin-1-yl]-butyl}-benzamide;
4-(4-chloro-benzoylamino)-N-{4-4-(2-ethoxy-4-methyl-phenyl)-piperidin-1-yl]butyl}-benzamide;
4-(4-chloro-benzoylamino)-N-{4-[4-(2-ethoxy-4-ethyl-phenyl)-pipeddin-1-yl]-butyl}-benzamide;
4-(4-chloro-benzoylamino)-N-{4-[4-(4-ethyl-2-methoxy-phenyl)-piperidin-1-yl]-butyl}-benzamide;
4-(4-chloro-benzoylamino)-N-{4-[4-(4-isopropyl-2-methoxy-phenyl)-piperidin-1-yl]-butyl}-benzamide;
4-(4-chloro-benzoylamino)-N-{4-[4-(2-ethoxy-4-isopropyl-phenyl)-piperidin-1-yl]-butyl}-benzamide;
4′-Trifluoromethyl-biphenyl-4-carboxylic acid (4-{4-[2,5-dimethyl-4-(pyridin-2-ylmethoxy)-phenyl]-piperidin-1-yl}-butyl)-amide;
4-(4-chloro-benzoylamino)-N-[4-(4-benzo[1,3]dioxol-5-yl-piperidin-1-yl]-butyl}-benzamide;
4-(4-chloro-benzoylamino)-N-[4-(4-naphthalen-2-yl-piperidin-1-yl]-butyl}-benzamide;
4-(4-chloro-benzoylamino)-N{4-[4-(5,6,7,8-tetrahydro-naphthalen-2-yl)-piperidin-1-yl]-butyl}-benzamide;
4-(4-chloro-benzoylamino)-N-[4-(4-naphthalen-1-yl-piperidin-1-yl]-butyl}-benzamide;
4-(4-chloro-benzoylamino)-N-{4-[4-(2-trifluoroethoxy-4-methyl-phenyl)-piperidin-1-yl]-butyl}-benzamide;
4′-Trifluoromethyl-biphenyl-4-carboxylic acid {4-[4-(2-methylsulfanyl-phenyl)-piperidin-1-yl]-butyl}-amide;
4′-Trifluoromethyl-biphenyl-4-carboxylic acid {4-[4-(1-methyl-1H-indol-3-yl)-piperidin-1-yl]-butyl}-amide;
4′-Trifluoromethyl-biphenyl-4-carboxylic acid {4-[4-(1H-indol-3-yl)-piperidin-1-yl]-butyl}-amide;
4′-Trifluoromethyl-biphenyl-4-carboxylic acid [4-(4-benzo[b]thiophen-3-yl-piperidin-1-yl)-butyl]-amide;
4-(4-chloro-benzoylamino)-N-4-[4-(2,4-dimethoxy-phenyl)-piperazin-1-yl]-butyl}-benzamide;
4-(4-Chloro-benzoylamino)-N-{4-[4-(2,4-dimethoxy-phenyl)-[1,4]diazocan-1-yl]-butyl}-benzamide;
4′-Trifluoromethyl-biphenyl-4-carboxylic acid {4-[4-(2-ethoxy-4-methyl-phenylamino)-piperidin-1-yl]-butyl}-amide;
4′-Trifluoromethyl-biphenyl-4-carboxylic acid (4-{4-[benzenesulfonyl-(2-ethoxy-4-methyl-phenyl)-amino]-piperidin-1-yl}-butyl)-amide;
4′-Trifluoromethyl-biphenyl-4-carboxylic acid {4-[4-(naphtalen-1-yloxy)-piperidin-1-yl]-butyl}-amide;
4-(4-chloro-benzoylamino)-N-{4-[4-(2-methoxy-4-methyl-phenyl)-piperazin-1-yl]-butyl}-benzamide;
4′-Trifluoromethyl-biphenyl-4-sulfonic acid {4-[4-(2-ethoxy-4-methyl-phenyl)-piperidin-1-yl]-butyl}-amide;
5-[4-(2-Ethoxy-4-methyl-phenyl)-piperidin-1-yl]-pentanoic acid (4′-trifluoromethyl-biphenyl-4-yl)-amide;
4′-{5-[4-(1-Methoxy-naphtalen-2-yl)-piperidin-1-yl]-pentyloxy}-biphenyl-4-carbonitrile;
4′-{4-[4-(1-Methoxy-naphtalen-2-yl)-piperidin-1-yl]-butoxy}-biphenyl-4-carbonitrile;
4-Methyl-2-(4-trifluoromethyl-phenyl)-thiazole-5-carboxylic acid {4-[4-(4-isopropyl-2-methoxy-phenyl)-piperidin-1-yl]-butyl}-amide;
2-(4-Chloro-phenyl)-1-methyl-1H-indole-5-carboxylic acid {4-[4-(2-ethoxy-4-methyl-phenyl)-piperidin-1-yl]-butyl}-amide;
2-(4-Trifluoromethyl-phenyl)-benzofuran-5-carboxylic acid {4-[4-(2-ethoxy-4-methyl-phenyl)-piperidin-1-yl]-butyl}-amide;
2-(4-Chloro-phenyl)-benzofuran-5-carboxylic acid {4-[4-(2-ethoxy-4-methyl-phenyl)-piperidin-1-yl]-butyl}-amide;
2-(3,4-Dichloro-phenyl)-benzofuran-5-carboxylic acid {4-[4-(1-cyclopropylmethoxy-5,6,7,8-tetrahydro-naphtalen-2-yl)-piperidin-1-yl]-butyl}-amide;
2-(6-Trifluoromethyl-pyridin-3-yl)-benzofuran-5-carboxylic acid {4-[4-(1-cyclopropylmethoxy-5,6,7,8-tetrahydronaphtalen-2-yl)-piperidin-1-yl]-butyl}-amide;
N-{4-[4-(2-Ethoxy-4-methyl-phenyl)-piperidin-1-yl]-butyl}4-[2-(4-trifluoromethyl-phenyl)-vinyl]-benzamide;
N-{4-[4-(2-Ethoxy-4-methyl-phenyl)-piperidin-1-yl]-butyl)-4-(4-trifluoromethyl-benzyloxy)-benzamide;
4-[2-(3,5-dichloro-phenyl)-ethenyl]-N-4-[4-(2,4-dimethoxy-phenyl)-piperazin-1-yl]-butyl}-benzamide;
4-[2-(3,5-dichloro-phenyl)-ethyl]-N-{4-[4-(2,4-dimethoxy-phenyl)-piperazin-1-yl]-butyl}-benzamide;
4-(4-Benzoyl)-N-{4-[4-(4-isopropyl-2-methoxy-phenyl)-piperidin-1-yl]-butyl}-benzamide;
4′-trifluoromethyl-biphenyl-4-carboxylic acid {4-[4-(2,4-diethoxy-benzyl)-piperidin-1-yl]-butyl}-amide;
4-(4-chloro-benzoylamino)-N-{4-[4-(2,4-dimethoxy-benzoyl)-piperidin-1-yl]-butyl}-benzamide;
4′-Cyano-biphenyl-4-carboxylic acid {4-[4-(1-methyl-1H-indol-3-yl)-piperidin-1-yl]-butyl}-amide;
4(4-chloro-benzoylamino)-N-{4-[4-(5-methyl-2-piperidin-4-yl-phenol)]-butyl}-benzamide;
1-(4-chloro-benzoylamino)-N-{4-[4-(5-ethyl-2-piperidin-4-yl-phenol)]-butyl}-benzamide;
4-(4-Chloro-benzoylamino)-N-{4-[4-(1-hydroxy-5,6,7,8-tetrahydro-naphtalen-2-yl)-piperidin-1-yl]-butyl}-benzamide;
4-(4-Chloro-benzoylamino)-N-(4-[4-(1-hydroxy-naphtalen-2-yl)-piperidin-1-yl]-butyl}-benzamide;
or a physiologically acceptable salt, solvate or derivative thereof, in the manufacture of a medicament for the treatment of diseases ameliorated by LDL-r upregulation.

13. A compound of formula (IC) 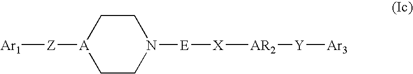
wherein
Ar₁ represents
(i) phenyl, naphthyl, or phenyl fused by a C₃₋₈cycloalkyl,
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently-saturated, partially unsaturated, or aromatic, providing that at least one ring is aromatic,
where Ar₁ optionally optionally bears 1-4 groups independently represented by R¹;
R¹ is selected from halogen, —S(C₁₋₄ alkyl), —O—(C₀₋₄ alkylene)-R² or C₀₋₄alkylene)-R², where each alkylene group may additionally incorporate an oxygen in the chain, with the proviso that there are at least two carbon atoms between any chain heteroatoms;
R² represents
(i) hydrogen, C₁₋₄ perfluoroalkyl, C₁₋₄perfluoroalkoxy,
(ii) phenyl, phenyl fused by a C₃₋₈ cycloalkyl, naphthyl or a 5- or 6-membered heteroaromatic group, optionally substituted by on or two groups independently selected from halogen, C₁₋₄ alkyl, hydroxy, C₁₋₄ alkoxy, amino, C₁₋₄ alkylamino and di-C₁₋₄ alkylamino,
(iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-C₁₋₄ alkylamino, or
(iv) amino, C₁₋₄ alkylamino or di-C₁₋₄alkylamino,
with the proviso that there are at least two carbon atoms between any chain heteroatoms;
Z is a direct link, oxo, —O—, C(H)R³, —N(R⁵)—, —N(SO₂R⁶)— or —SO₂—;
R³ is hydrogen, C₁₋₄ alkyl or phenyl, said phenyl optionally bearing one or two groups indpendently selected from halogen, C₁₋₄ alkyl, CIA alkoxy and OH;
A is C—R⁴ or N;
E represents a C₄₋₅alkylene group;
R⁴ is hydrogen, C₁₋₄ alkyl, hydroxy or phenyl, said phenyl optionally bearing one or two groups indpendently selected from halogen, C₁₋₄ alkyl, C₁₋₄ alkoxy or hydroxy, or R³ forms a double bond between A and an adjacent ring carbon;
R⁵ is C₁₋₄ alkyl or phenyl;
R⁶ is C₁₋₄ alkyl or phenyl;
X is a bond, —O—, oxo, —CON(H or C₁₋₄ alkyl)-, —N(H or C₁₋₄ alkyl)CO—, —N(H or C₁₋₄ alkyl)SO₂— or —SO₂N(H or C₁₋₄alkyl)-;
Ar₂ is phenyl, a 5-6 membered heteroaromatic group or fused bicyclic aromatic radicals, wherein said radicals contain a total of from 8-12 ring atoms, wherein said radicals contain a total of from 1-4 ring heteroatoms independently selected from oxygen, nitrogen and sulfur, where each group is optionally substituted by one or two groups independently selected from C₁₋₄ alkyl, halogen, hydroxy, C₁₋₄ alkoxy, C₁₋₆ acyl, C₁₋₄acyloxy, amino, C₁₋₄ alkylamino and di-C₁₋₄ alkylamino groups;
Y is a bond, oxo, —O—, —N(H or C₁₋₄ alkyl)CO—, —CON(H or C₁₋₄ alkyl)-, —N(H or C₁₋₄ alkyl)SO₂— or —SO₂N(H or C₁₋₄ alkyl)-, —C₁₋₂ alkylene-, —O—C₁₋₂ alkylene- or —C₂₋₃alkenylene-;
Ar₃ represents
(i) phenyl, naphthyl, or phenyl fused by a C₃₋₈cycloalkyl,
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic, where Ar₃ optionally bears 1-4 groups independently selected from halogen, nitrile, C₁₋₄ alkyl, C₁₋₄ alkoxy, C₂₋₄ alkenyl, C₂₋₄ alkenyloxy, hydroxy, azido, C₁₋₄perfluoroalkyl, C₁₋₄perfluoroalkoxy, nitro, CIA alkylsulfonyl, C₁₋₄ alkylaminosulfonyl, C₁₋₄ dialkylaminosulfonyl, C₁₋₄ acyl, C₁₋₄ acyloxy, C₁₋₄ alkoxycarbonyl, aminocarbonyl, C₁₋₄ alkylaminocarbonyl, di-C₁₋₄ alkylaminocarbonyl, C₁₋₄ acylamino, amino, C₁₋₄ alkylamino or di-C₁₋₄ alkylamino groups;
or a physiologically acceptable salt, solvate or derivative thereof, with the proviso that compounds of formula (A) are excluded 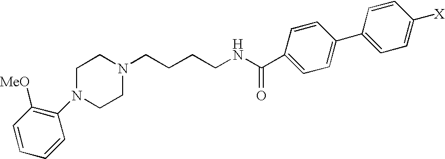
where X may be COMe, SO₂Me and NH₂.

14. Use of a compound according to claim 13 in human medicine.

15. Use of a compound according to claim 13 or a physiologically acceptable salt solvate or derivative thereof in the preparation of a medicament for use in'the treatment of conditions resulting from elevated circulating levels of LDL-cholesterol.

16. A method for the treatment of a mammal, including man, of conditions resulting from elevated circulating levels of LDL-cholesterol, comprising administration of an effective amount of a compound according to claim 13 or a physiologically acceptable salt or solvate thereof.

17. A pharmaceutical composition which comprises at least one compound according to claim 13 or a physiologically acceptable-salt solvate or derivative thereof, with one or more pharmaceutically acceptable carriers or excipients and optionally one or more further physiologically active agents.

18. A process for the preparation of compound of formula (Ib) comprising:
(A) reaction of a compound of formula (II) with a compound of formula (III) 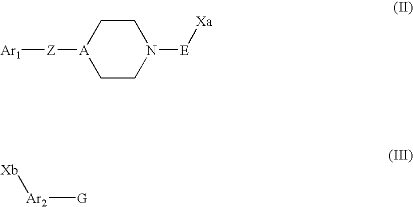
where Xa and Xb are suitable reactants to form a group X;
(B) reaction of a compound of formula (IV) with a compound of formula (XIII) 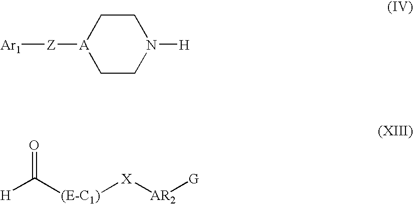
where E-C₁ (‘E minus C₁’) means that the chain length of group E is one carbon less than that in the resulting compound (I), under standard reductive amination conditions; or
(C) reaction of a different compound of formula (I).
